(19) United States
(12) Patent Application Publication
(10) Pub. No.: US 2025/0213721 A1
(43) Pub. Date: Jul. 3, 2025

(54) COMPOSITIONS INCLUDING IFNE AND USES THEREOF

(71) Applicants: Memorial Sloan-Kettering Cancer Center, New York, NY (US); Memorial Hospital for Cancer and Allied Diseases, New York, NY (US); Sloan-Kettering Institute for Cancer Research, New York, NY (US)

(72) Inventors: Francisco M. Barriga, New York, NY (US); Scott Lowe, New York, NY (US); Kaloyan Tsanov, New York, NY (US)

(21) Appl. No.: 18/849,996

(22) PCT Filed: Mar. 23, 2023
(86) PCT No.: PCT/US2023/016150

§ 371 Date: Sep. 23, 2024

Related U.S. Application Data
(60) Provisional application No. 63/323,319, filed on Mar. 24, 2022.
(60) Provisional application No. 63/323,207, filed on Mar. 24, 2022.

(51) Int. Cl. A61K 48/00; A61K 38/21; A61K 45/06; A61P 35/00; C12N 9/22; C12N 15/11; C12N 15/90

(52) U.S. Cl. CPC A61K 48/005; A61K 38/21; A61K 45/06; A61P 35/00; C12N 9/22; C12N 15/111; C12N 15/907; C12N 2310/20

(57) ABSTRACT

The present disclosure provides compositions comprising IFNE and methods of using the same to treat cancer and/or enhancing responsiveness to immune checkpoint blockade therapy in a patient in need thereof. Also disclosed herein are compositions including tandem bicistronic expression cassettes, and methods of using the same to generate large genomic deletions and/or knock-in gene alterations.

### CROSS-REFERENCE TO RELATED APPLICATIONS

This application is the U.S. National Stage Application of International Application No. PCT/US2023/016150, filed Mar. 23, 2023, which claims the benefit of and priority to U.S. Provisional Patent Application No. 63/323,319, filed Mar. 24, 2022, and U.S. Provisional Patent Application No. 63/323,207, filed Mar. 24, 2022, the contents of which are incorporated herein by reference in their entireties.

### STATEMENT OF GOVERNMENT SUPPORT

This invention was made with government support under CA008748 awarded by the National Institutes of Health. The government has certain rights in the invention.

### TECHNICAL FIELD

The present technology relates generally to compositions comprising IFNE and methods of using the same to treat cancer and/or enhancing responsiveness to immune checkpoint blockade therapy in a patient in need thereof. Also provided herein are compositions including tandem bicistronic expression cassettes, and methods of using the same to generate large genomic deletions and/or knock-in gene alterations.

### BACKGROUND

The following description of the background of the present technology is provided simply as an aid in understanding the present technology and is not admitted to describe or constitute prior art to the present technology.

A large class of cancer-associated lesions are copy number alterations (CNAs), which simultaneously impact the dosage of multiple genes and include chromosomal gains and losses, focal amplifications, and heterozygous or homozygous deletions^{1,2}. Current estimates suggest that a typical tumor carries an average of 24 distinct CNAs that impact up to 30% of the genome^{3,4,6}. Moreover, CNAs show recurrent patterns that can be associated with clinical outcomes^{3,4,7,8}, arguing for active selection of specific traits rather than stochastic accumulation of genomic alterations. While much of the research on CNAs has focused on known drivers within the affected regions, emerging evidence indicates that co-gained or co-deleted genes-once considered “passenger” events—can also contribute to tumorigenesis^{1,9,10}. While these observations imply CNAs produce complex phenotypes that cannot be recapitulated by manipulating a single gene^{11,17}, the experimental modeling of these lesions remains a major challenge that has impeded the functional assessment of CNA biology^{12,13,18-21}.

Among recurrent CNAs, loss of chromosome 9p21.3 is most strongly linked to poor prognosis and the most common homozygous deletion across human cancers^{3,7}. The 9p21.3 locus is particularly prominent since it encompasses multiple key tumor suppressor genes (TSGs): the cell cycle inhibitors (DKN2A (encoding p16^{INK4a} and p14^{ARF}) and (DKN2B (encoding p15^{INK4b}), which collectively engage the function of p53 and RB, the major tumor-suppressive pathways that are impaired in cancer^{5,22-24}. Tumors with 9p21.3 deletions can display altered immune infiltrates^{25,26} and increased resistance to immune checkpoint blockade^{27,28}, suggesting that the locus may also influence immune-related processes.

Accordingly, there is an urgent need for therapeutic agents that effectively treat cancer patients harboring cancer-associated copy number alterations.

### SUMMARY OF THE PRESENT TECHNOLOGY

In one aspect, the present disclosure provides a method for treating cancer in a patient in need thereof comprising administering to the patient an effective amount of IFNE, wherein the patient comprises focal deletions in Cdkn2a and Cdkn2b. Also provided herein is a method for enhancing responsiveness to immune checkpoint blockade therapy in a cancer patient in need thereof comprising administering to the patient an effective amount of IFNE and an effective amount of an immune checkpoint inhibitor, wherein the patient comprises focal deletions in Cdkn2a and Cdkn2b. In some embodiments, the immune checkpoint inhibitor is an anti-PD-1 antibody, an anti-PD-L1 antibody, an anti-PD-L2 antibody, an anti-CTLA-4 antibody, an anti-TIM3 antibody, an anti-4-1BB antibody, an anti-CD73 antibody, an anti-GITR antibody, or an anti-LAG-3 antibody. Additionally or alternatively, in some embodiments, the immune checkpoint inhibitor may be selected from among CTLA4 (for example, Yervoy (ipilimumab), CP-675,206 (tremelimumab), AK104 (cadonilimab), or AGEN1884 (zalifrelimab)), or an antibody or an equivalent thereof recognizing and binding to PD-1 (for example, Keytruda (pembrolizumab), Opdivo (nivolumab), Libtayo (cemiplimab), Tyvyt (sintilimab), BGB-A317 (tislelizumab), JS001 (toripalimab), SHR1210 (camrelizumab), GB226 (geptanolimab), JS001 (toripalimab), AB122 (zimberelimab), AK105 (penpulimab), HLX10 (serplulimab), BCD-100 (prolgolimab), AGEN2034 (balstilimab), MGA012 (retifanlimab), AK104 (cadonilimab), HX008 (pucotenlimab), PF-06801591 (sasanlimab), JNJ-63723283 (cetrelimab), MGD013 (tebotelimab), CT-011 (pidilizumab), or Jemperli (dostarlimab)), or an antibody or an equivalent thereof recognizing and binding to PD-L1 (for example, Tecentriq (atezolizumab), Imfinzi (durvalumab), Bavencio (avelumab), CS1001 (sugemalimab), or KN035 (envafolimab)).

Additionally or alternatively, in some embodiments, the focal deletions in Cdkn2a and Cdkn2b are no more than 0.4 Mb, no more than 0.3 Mb, no more than 0.2 Mb, no more than 0.1 Mb, no more than 90 Kb, no more than 80 Kb, no more than 70 Kb, no more than 60 Kb, no more than 50 Kb, no more than 40 Kb, no more than 30 Kb, no more than 20 Kb, no more than 10 Kb, no more than 9 Kb, no more than 8 Kb, no more than 7 Kb, no more than 6 Kb, no more than 5 Kb, no more than 4 Kb, no more than 3 Kb, no more than 2 Kb, or no more than 1 Kb in length.

In some embodiments of the methods disclosed herein, the patient further comprises deletions in at least one IFN gene in type I IFN cluster. The type I IFN cluster may comprise IFN-α1, IFN-α2, IFN-α4, IFN-α5, IFN-α6, IFN-α7, IFN-α8, IFN-α10, IFN-α13, IFN-α14, IFN-α16, IFN-α17, IFN-α21, IFNB, IFN-Epsilon, IFN-Kappa, and IFN-Omega. In certain embodiments, deletions in the type I IFN cluster are no more than 1.3 Mb, no more than 1.2 Mb, no more than 1.1 Mb, no more than 1 Mb, no more than 0.9 Mb, no more than 0.8 Mb, no more than 0.7 Mb, no more than 0.6 Mb, no more than 0.5 Mb, or no more than 0.4 Mb in length.

In any and all embodiments of the methods disclosed herein, the cancer may be selected from among lung cancer, pancreatic cancer, head and neck squamous cell cancer, esophageal carcinoma, skin cutaneous melanoma, stomach cancer, glioblastoma, bladder urothelial carcinoma, or brain lower grade glioma. In some embodiments, the pancreatic cancer is pancreatic adenocarcinoma (PDAC). In other embodiments, the lung cancer is lung adenocarcinoma (LUAD) or lung squamous cell carcinoma.

In any and all embodiments of the methods disclosed herein, the IFNE is administered orally, topically, intranasally, systemically, intravenously, subcutaneously, intraperitoneally, intradermally, intraocularly, iontophoretically, transmucosally, or intramuscularly. Additionally or alternatively, in some embodiments, the IFNE comprises, consists essentially of, or consists of an amino acid sequence that is at least 80%, 85%, 90%, 95%, 96%, 97%, 98%, or 99% identical to SEQ ID NO: 64.

In one aspect, the present disclosure provides a donor nucleic acid template including a bicistronic expression cassette comprising a first cistron and a second cistron that are tandemly located, wherein the first cistron encodes a positive selection marker and the second cistron encodes a negative selection marker. The second cistron may be located at the 5′ end or the 3′ end of the first cistron. Additionally or alternatively, in some embodiments, a first artificial protospacer sequence is located upstream of the bicistronic expression cassette and/or a second artificial protospacer sequence is located at downstream of the bicistronic expression cassette. In certain embodiments, an Internal Ribosome Entry Site (IRES) sequence or a 2A peptide sequence is interspersed between the first cistron and the second cistron. The 2A peptide sequence may comprise any one of SEQ ID NOs: 59-62.

Additionally or alternatively, in some embodiments, the donor nucleic acid template further comprises a heterologous nucleic acid encoding an enzyme, a bioluminescent protein, a fluorescent protein, and/or a chemiluminescent protein, wherein the heterologous nucleic acid is located upstream or downstream of the bicistronic expression cassette. Fluorescent proteins include, but are not limited to, blue/UV fluorescent proteins (for example, TagBFP, Azurite, EBFP2, mKa1ama1, Sirius, Sapphire, and T-Sapphire), cyan fluorescent proteins (for example, ECFP, Cerulean, SCFP3A, m Turquoise, monomeric Midoriishi-Cyan, TagCFP, and mTFP1), green fluorescent proteins (for example, EGFP, Emerald, Superfolder GFP, Monomeric Azami Green, TagGFP2, mUKG, and mWasabi), yellow fluorescent proteins (for example, EYFP, Citrine, Venus, SYFP2, and TagYFP), orange fluorescent proteins (for example, Monomeric Kusabira-Orange, mKOK, mKO2, mOrange, and mOrange2), red fluorescent proteins (for example, mRaspberry, mCherry, dsRed, mStrawberry, mTangerine, tdTomato, TagRFP, TagRFP-T, mApple, and mRuby), far-red fluorescent proteins (for example, mPlum, HcRed-Tandem, mKate2, mNeptune, and NirFP), near-IR fluorescent proteins (for example, TagRFP657, IFP1.4, and iRFP), long stokes-shift proteins (for example, mKeima Red, LSS-mKate1, and LSS-mKate2), photoactivatable fluorescent proteins (for example, PA-GFP, PAmCherry 1, and PATagRFP), photoconvertible fluorescent proteins (for example, Kaede (green), Kaede (red), KikGR1 (green), KikGR1 (red), PS-CFP2, PS-CFP2, mEos2 (green), mEos2 (red), PSmOrange, and PSmOrange), fluorescein, rhodamine, and photoswitchable fluorescent proteins (for example, Dronpa). Examples of bioluminescent proteins are aequorin (derived from the jellyfish Aequorea victoria) and luciferases (including luciferases derived from firefly and Renilla, nanoluciferase, red luciferase, luxAB, and the like). Examples of chemiluminescent protein include β-galactosidase, horseradish peroxidase (HRP), and alkaline phosphatase.

Additionally or alternatively, in some embodiments, the bicistronic expression cassette is operably linked to an inducible promoter or a constitutive promoter. Examples of constitutive promoters include CMV, EF1a, SV40, PGK1, Ubc, human beta actin, CAG, Ac5, Polyhedrin, TEF1, GDS, ADH1 (repressed by ethanol), CaMV35S, Ubi, H1, U6, T7 (requires T7 RNA polymerase), and SP6 (requires SP6 RNA polymerase). Examples of inducible promoters include TRE (inducible by Tetracycline or its derivatives; repressible by TetR repressor), GAL1 & GAL10 (inducible with galactose; repressible with glucose), lac (constitutive in the absence of lac repressor (LacI); can be induced by IPTG or lactose), T7lac (hybrid of T7 and lac; requires T7 RNA polymerase which is also controlled by lac operator; can be induced by TRIG or lactose), araBAD (inducible by arabinose which binds repressor AraC to switch it to activate transcription; repressed catabolite repression in the presence of glucose via the CAP binding site or by competitive binding of the anti-inducer fucose), trp (repressible by tryptophan upon binding with TrpR repressor), tac (hybrid of lac and trp; regulated like the lac promoter; e.g., tad and tacII), and pL (temperature regulated).

Additionally or alternatively, in some embodiments, the positive selection marker is an antibiotic resistance gene. Examples of positive selection markers include, but are not limited to neomycin phosphotransferase, hygromycin phosphotransferase, phosphoinothricin acetyltransferase, glyphosate oxidoreductase, adenosine deaminase (ADA), aminoglycoside phosphotransferase, bleomycin, cytosine deaminase, dihydrofolate reductase, histidinol dehydrogenase, puromycin-N-acetyl transferase, thymidine kinase, or xanthine-guanine phosphoribosyltransferase.

Examples of negative selection markers include, but are not limited to herpes simplex virus thymidine kinase (HSV-TK), mlA, ypjF, ykfl, ydaS, yjhX, relE, mqsR, toxin CcdB, levansucrase, cytosine deaminase, or diphtheria toxin A (DT-A).

In one aspect, the present disclosure provides a method for knocking in a genetic alteration at a target gene locus in cells comprising: (a) contacting cells with a sgRNA-CRISPR enzyme conjugate in vivo under conditions where the sgRNA-CRISPR enzyme conjugate cleaves an endogenous protospacer sequence at the target gene locus in the cells to produce a cleaved target gene locus; (b) integrating the donor nucleic acid template of the present technology into the cleaved target gene locus via CRISPR-facilitated homology-directed repair, wherein the donor nucleic acid template comprises a 5′ flanking region and a 3′ flanking region that are homologous to the target gene locus; (c) enriching cells that stably express the positive selection marker; (d) contacting the enriched cells of step (c) with a first sgRNA-CRISPR enzyme complex and a second sgRNA-CRISPR enzyme complex in vivo under conditions where the first sgRNA-CRISPR enzyme complex cleaves the first artificial protospacer sequence within the donor nucleic acid template and the second sgRNA-CRISPR enzyme complex cleaves the second artificial protospacer sequence within the donor nucleic acid template to delete the bicistronic expression cassette; and (e) eliminating cells that stably express the negative selection marker to obtain a cell population comprising the genetic alteration at the target gene locus.

In another aspect, the present disclosure provides a method for knocking in a genetic alteration at a target gene locus in cells comprising: (a) contacting cells with a first sgRNA-CRISPR enzyme conjugate in vivo under conditions where the sgRNA-CRISPR enzyme conjugate cleaves a first endogenous protospacer sequence at the target gene locus in the cells to produce a cleaved target gene locus; (b) integrating the donor nucleic acid template of the present technology into the cleaved target gene locus via CRISPR-facilitated homology-directed repair, wherein the donor nucleic acid template comprises a 5′ flanking region and a 3′ flanking region that are homologous to the target gene locus; (c) enriching cells that stably express the positive selection marker; (d) contacting the enriched cells of step (c) with a second sgRNA-CRISPR enzyme complex and a third sgRNA-CRISPR enzyme complex in vivo under conditions where the second sgRNA-CRISPR enzyme complex cleaves the first artificial protospacer sequence within the donor nucleic acid template and the third sgRNA-CRISPR enzyme complex cleaves a second endogenous protospacer sequence at the target gene locus to delete the bicistronic expression cassette, wherein the second endogenous protospacer sequence is located downstream of the bicistronic expression cassette; and (e) eliminating cells that stably express the negative selection marker to obtain a cell population comprising the genetic alteration at the target gene locus.

Additionally or alternatively, in some embodiments of the methods disclosed herein, the homologous 5′ flanking region of the donor nucleic acid template has a length of about 20-30 base pairs (bps), 30-40 bps, 40-50 bps, 50-60 bps, 60-70 bps, 70-80 bps, 80-90 bps, 90-100 bps, 100-110 bps, 110-120 bps, 120-130 bps, 130-140 bps, 140-150 bps, 150-160 bps, 160-170 bps, 170-180 bps, 180-190 bps, 190-200 bps, 200-210 bps, 210-220 bps, 220-230 bps, 230-240 bps, 240-250 bps, 250-260 bps, 260-270 bps, 270-280 bps, 280-290 bps, 290-300 bps, 300-310 bps, 310-320 bps, 320-330 bps, 330-340 bps, 340-350 bps, 350-360 bps, 360-370 bps, 370-380 bps, 380-390 bps, 390-400 bps, 400-410 bps, 410-420 bps, 420-430 bps, 430-440 bps, 440-450 bps, 450-460 bps, 460-470 bps, 470-480 bps, 480-490 bps, 490-500 bps, 500-510 bps, 510-520 bps, 520-530 bps, 530-540 bps, 540-550 bps, 550-560 bps, 560-570 bps, 570-580 bps, 580-590 bps, or 590-600 bps.

Additionally or alternatively, in some embodiments of the methods disclosed herein, the homologous 3′ flanking region of the donor nucleic acid template sequence has a length of about 20-30 base pairs (bps), 30-40 bps, 40-50 bps, 50-60 bps, 60-70 bps, 70-80 bps, 80-90 bps, 90-100 bps, 100-110 bps, 110-120 bps, 120-130 bps, 130-140 bps, 140-150 bps, 150-160 bps, 160-170 bps, 170-180 bps, 180-190 bps, 190-200 bps, 200-210 bps, 210-220 bps, 220-230 bps, 230-240 bps, 240-250 bps, 250-260 bps, 260-270 bps, 270-280 bps, 280-290 bps, 290-300 bps, 300-310 bps, 310-320 bps, 320-330 bps, 330-340 bps, 340-350 bps, 350-360 bps, 360-370 bps, 370-380 bps, 380-390 bps, 390-400 bps, 400-410 bps, 410-420 bps, 420-430 bps, 430-440 bps, 440-450 bps, 450-460 bps, 460-470 bps, 470-480 bps, 480-490 bps, 490-500 bps, 500-510 bps, 510-520 bps, 520-530 bps, 530-540 bps, 540-550 bps, 550-560 bps, 560-570 bps, 570-580 bps, 580-590 bps, or 590-600 bps.

In any and all embodiments of the methods disclosed herein, the cells are mammalian cells, embryonic stem cells, or fibroblasts.

### BRIEF DESCRIPTION OF THE DRAWINGS

FIGS. 1A-1H: MACHETE Enables Efficient Engineering of Genomic Deletions. FIG. 1A: Schematic of the MACHETE approach. FIG. 1B: Frequency of homozygous deletions across the pan-cancer TCGA dataset. FIG. 1C: Relative frequency of deletions at the 9p21.3 locus classified as 9pS and 9 pL across different cancer types. FIG. 1D: Frequency of deep deletion of 9p21.3 genes in PDAC patients. FIG. 1E: Schematic of MACHETE-mediated engineering of 4C4 ΔS and ΔL deletions. FIG. 1F: PCR genotyping for the WT, KI, ΔS and ΔL alleles in the indicated PDEC cell lines. FIG. 1G: Pattern of resistance/sensitivity to positive and negative selection in PDEC sgP53 EL parental, 4C4 KI, AS, and ΔL cells. Cells were seeded and treated with Puromycin (2 mg/mL) or DT-A (50 ng/mL) for 72 hours, and then stained with crystal violet to assess surviving cells. FIG. 1H: DNA sequencing of breakpoints from ΔS and ΔL cells confirming loss of the expected genomic regions (0.4 Mb deletion in AS, and 1.3 Mb deletion in AL).

FIGS. 2A-2L: ΔL Deletions Are Differentially Surveilled by the Adaptive Immune System and Promote Metastasis. FIG. 2A: Engraftment at one month after injection of ΔS and ΔL cells in C57BL/6, nude, and NSG hosts. Two independently generated input cell lines were used per genotype (n≥5 per each cell line). Bars represent fraction of metastasis-bearing mice (specific numbers of independently analyzed mice are noted in parentheses). ns=non-significant (chi-square test). FIG. 2B: Representative macroscopic fluorescent images of primary tumors harvested from the indicated genotypes and hosts. Insets show the brightfield image for each tumor. FIG. 2C: qPCR analysis for EGFP copy number in the gDNA of tumor-derived (Post in vivo) ΔS and ΔL lines from C57BL/6 and Nude hosts, relative to their parental (Pre in vivo) counterparts. Each dot represents an independent tumor-derived cell line. ** p<0.01, ns=non-significant, two-tailed t-test. FIG. 2D: Representative images of metastases in C57BL/6 mice with AL tumors. Left: Brightfield macroscopic images of abdominal (intestinal and mesenteric lymph node) metastases. Insets show matched EGFP fluorescence images. Middle: Macroscopic and Hematoxylin/Eosin images of tumor-bearing livers. Right: Macroscopic and Hematoxylin/Eosin images of tumor-bearing lungs. T=tumor; N=normal adjacent tissue. FIGS. 2E-2F: Overall (FIG. 2E) and organ-specific (FIG. 2F) metastasis incidence in C57BL/6 mice with either ΔS or ΔL tumors. 4 independently generated input cell lines were used per genotype (n≥5 per each cell line). Bars represent fraction of metastasis-bearing mice (specific numbers of independently analyzed mice are noted in parentheses). *p<0.05; *** p<0.001, chi-square test. FIG. 2G: Representative images of metastases in Nude mice with ΔL or ΔS tumors. Hematoxylin/Eosin images of tumor-bearing livers (left) and lungs (right) are shown. FIGS. 2H-2I: Overall (FIG. 2H) and organ-specific (FIG. 2I) metastasis incidence in C57BL/6 mice with either ΔS or ΔL tumors. 4 independently generated input cell lines were used per genotype (n≥5 per each cell line). Bars represent fraction of metastasis-bearing mice (specific numbers of independently analyzed mice are noted in parentheses). ns=non-significant, chi-square test. FIG. 2J: Representative gross morphology (top) and Hematoxylin/Eosin histological stain (bottom) of matched primary tumor and overt liver metastasis in a Kras^{G12D/+}; shSmad4 PDAC GEMM. FIG. 2K: sWGS analysis of tumor-derived cell lines from the KC-Ren and KC-Smad4 GEMMs, grouped by spontaneous 4C4 deletion type (WT, ΔS, ΔL). Schematic of the murine 4C4 locus is shown on top. Blue tracks indicate deleted regions, with color intensity corresponding to the extent of the deletion. Numbers correspond to independent mice. FIG. 2L: Incidence of overt metastasis in mice with tumors that harbor WT 4C4 locus, ΔS or ΔL deletions. Bars represent fraction of metastasis-bearing mice (specific numbers of independently analyzed mice are noted in parentheses). * p<0.05, chi-square test.

FIGS. 3A-3K: 4C4/9p21.3 Deletion Genotype Dictates Type I IFN Signaling and Immune Infiltration. FIG. 3A: UMAP of CD45+ cells showing cells derived from ΔS (n=7774 cells) or ΔL (n=7560 cells) tumors. FIG. 3B: UMAP of CD45+ cells annotating the specific immune subsets. FIG. 3C: UMAP of averaged IFN response signature across CD45+ populations. FIG. 3D: (Upper) UMAP of CD8+ T cells from ΔS or ΔL tumors. Cells are colored by sample. (Bottom) UMAP of CD8+ T cell clusters. Cells are colored and by their cluster identity. FIG. 3E: UMAP of imputed expression for the indicated genes. FIG. 3F: MILO analysis of CD8+ T cells. Neighborhoods identified through MILO analysis using default parameters (red indicates enrichment in AS, while blue indicates enrichment in AL). FIG. 3G: Swarm plot of the distribution of CD8+ T cell neighborhoods in ΔS or ΔL tumors across transcriptional states. The x-axis indicates the Log-fold change in differential abundance of ΔS (<0) and ΔL (>0). Each neighborhood is associated with a cell type if more than 80% of the cell state in the neighborhood belong to said state, else is annotated as “Mixed”. FIG. 3H: Differential gene expression of the indicated genes in Pdcd1+Mki67-CD8+ T cells. FIG. 3I: UMAP of imputed expression of Tox and Bcl2. Dashed circles highlight AS-enriched CD8+ T cells. FIG. 3J: Representative images of liver metastasis upon CD8+ cell depletion. FIG. 3K: Incidence of metastasis upon depletion of immune subsets in ΔS or ΔL tumors. 2 independently generated input cell lines were used per genotype (n≥5 per each cell line). Bars represent fraction of metastasis-bearing mice (specific numbers of independently analyzed mice are noted in parentheses). ns=non-significant; ** p<0.01; *** p<0.001, chi-square test.

FIGS. 4A-4M: Ifne Is a Tumor-specific Mediator of Immune Surveillance and Metastasis. FIG. 4A: Quantification of EGFP fluorescence in ΔS or ΔL tumors from C57BL/6 mice treated with IgG or aIFNARI antibodies. Representative plots are shown in FIG. 11B. Each dot represents an independent biological replicate. * p<0.05, one-way ANOVA followed by Tukey's multiple comparison test. FIG. 4B: Incidence of metastasis in C57BL/6 mice transplanted with homozygous ΔS or ΔL lines and treated with IgG or aIFNARI antibodies. 2 independently generated input cell lines were used per genotype (n≥5 per each cell line). Bars represent fraction of mice bearing metastasis (total numbers of independently analyzed mice are shown). ns=non-significant; * p<0.05, chi-square test.

FIG. 4C: Volcano plots of differentially expressed genes comparing IFNAR1 blockade vs. IgG controls in ΔS or ΔL tumors. FIG. 4D: Schematic of extended series of 4C4 deletion alleles. FIG. 4E: (Left) Flow cytometry measurement of EGFP fluorescence in tumors derived from deletion series mix (“Mix”). EGFP-negative cells were used as negative controls (“Neg”). (Right) Schematic of in vivo competition experiment. FIG. 4F: Representative EGFP immunofluorescent stain of a deletion-mix tumor. FIG. 4G: (Left) Representative flow cytometry plot of EGFP levels in a deletion-mix tumor. GFP-Low and GFP-High cell populations were sorted as marked. (Right) Copy-number qPCR analysis of the indicated genes in the parental cell mix, and GFP-Low vs. GFP-High cells sorted from resulting tumors. FIG. 4H: Relative copy-number quantification of indicated genes in GFP-High vs. GFP-Low cells. * p<0.05; ns=non-significant, one-way ANOVA followed by Sidak's multiple comparison test. Bars represent SEM, n=7 biological replicates. FIG. 4I: Relative copy-number quantification of indicated genes in metastases-vs. primary tumor-derived cells. * p<0.05; *** p<0.001; ns=non-significant, one-way ANOVA followed by Sidak's multiple comparison test. Bars represent SEM, n=7 primary tumors and 6 metastases. FIG. 4J: RT-qPCR measurements of mRNA levels for the indicated IFN genes in tumor cells and infiltrating CD45+ cells from ΔS tumors. Each dot is a biological replicate (n=4). FIG. 4K: Relative quantification of primary tumor weights (left) and number of mesenteric LN metastases (right) in ΔS and ΔL tumors with add-back of Ifne-expressing or control construct. * p<0.05; ns-non-significant, one-way ANOVA followed by Sidak's multiple comparison test to the respective control population. Each dot is an independent tumor. FIG. 4L: Flow cytometry-based quantification of TAM fraction (left) and TAM MHCII levels (right) in tumors of the indicated genotypes. ** p<0.01; *** p<0.001; ns=non-significant, one-way ANOVA followed by Sidak's multiple comparison test to the respective control population. Each dot is an independent tumor.

FIG. 4M: Flow cytometry-based quantification of CD69 (left) and PD1 (right) levels in CD8+CD44+ T cells from tumors of the indicated genotypes. * p<0.05; *** p<0.001; ns-non-significant, one-way ANOVA followed by Sidak's multiple comparison test to the respective control population. Each dot is an independent tumor.

FIG. 5A: Preparation of donor DNA and sgRNA used for MACHETE-mediated targeting of the 11B3 locus in NIH3T3 cells. FIG. 5B: Experimental outline and timing for MACHETE-based 11B3 deletion engineering in NIH3T3 cells. FIG. 5C: Schematic of MACHETE-mediated engineering of a 4.1 Mb deletion at the 11B3 locus. FIG. 5D: Crystal violet stain of WT, 11B3 KI and D11B3 NIH3T3 cells after selection with puromycin (Puro, 2 mg/mL) and/or diphtheria toxin (DT-A, 50 ng/ml). FIG. 5E: PCR genotyping for the 11B3 KI and D11B3 alleles in the indicated NIH3T3 cell lines. FIG. 5F: (Left) Experimental outline for testing the impact of DT-mediated negative selection on the efficiency of D11B3 deletion engineering in NIH3T3 cells. (Right) Clonal analysis of NIH3T3 cells engineered without (−DT) and with (+DT)diphtheria toxin selection. FIG. 5G: Sanger sequencing of the 11B3 deletion breakpoint confirming the expected deletion. FIG. 5G discloses SEQ ID NOS 65-66, respectively, in order of appearance. FIG. 5H: Suite of dual selection cassettes generated for the MACHETE approach. FIG. 5I: Schematic of MACHETE-mediated engineering of a 45 Mb deletion at the 7q11-22 locus in HEK293 cells. FIG. 5J: Flow cytometry plots and quantification of BFP+ and BFP-HEK293 cells under the indicated conditions. FIG. 5K: PCR genotyping for the 7q11 KI and D7q11-22 alleles in HEK293 cells under the indicated conditions.

FIG. 6A: Frequency of deep deletions at the 9p21.3 locus across different types of cancer in the TCGA dataset. FIG. 6B: Mutation frequency of KRΔS and TP53 in 9 pL and 9pS PDAC patients in the TCGA dataset. FIG. 6C: Schematic of the synteny between the human 9p21.3 and mouse 4C4 locus. FIG. 6D: Schematic of the generation of PDEC sgP53 EL cells. CRISPR-mediated knockout of Trp53 was done by electroporation of a pX330-sgP53 plasmid followed by treatment with Nutlin-3 (10 mM) to select for Trp53-deficient cells. PDEC sgP53 cells were then infected with a retroviral EGFP-Luciferase construct and cells were selected by sorting for EGFP+expression. FIG. 6E: Clonal analysis of ΔS and ΔL cells engineered without (−DT) and with (+DT)diphtheria toxin selection. FIG. 6F: Frequency of heterozygous and homozygous ΔS or ΔL deletions in PDEC cells following MACHETE engineering. FIG. 6G: (Left) Schematic of iterative editing of cells bearing a heterozygous ΔL deletion, using a distinct set of guides to discern between the different deletions. (Right) PCR genotyping of the distinct ΔL deletion breakpoints. FIG. 6H: Histology of ΔS and ΔL tumors in C57BL/6 mice. Representative H/E images are shown. FIG. 6I: sWGS analysis of 4C4 deletion status in ΔS and AL tumor-derived cell lines (from C57BL/6 hosts). Deep blue color depicts deletion defined as log 2 relative abundance <−2. FIG. 6J: (Top) Schematic representation of the MACHETE-engineered ΔI allele that removes a 0.9 Mb region downstream of Hacd4 and upstream of Cdkn2a. (Bottom) Engraftment of ΔI cells in C57BL/6 mice one month after injection and measured by bioluminescence. FIG. 6K: (Left) Representative macroscopic image of a ΔI tumor showing retained EGFP expression at endpoint. Inset shows matched brighfield image. (Right) qPCR analysis for EGFP copy number in the gDNA of tumor-derived (Post in vivo) ΔI cell lines from C57BL/6 hosts relative to their parental (Pre in vivo) counterparts. Each dot represents an independent cell line. FIG. 6L: Survival curve of C57BL/6 mice transplanted with AS, AI, or ΔL tumor cells. Depicted are the number of mice transplanted and the median survival, which showed no statistically significant differences (logrank test).

FIG. 7A: EGFP levels of representative re-sorted tumor-derived ΔS and ΔL cell lines. FIG. 7B: Growth curves in adherent (top) or suspension (bottom) conditions for AS and ΔL cell lines. FIG. 7C: Macroscopic images (left) and hematoxylin/eosin stain (right) of orthotopic tumors in C57BL/6 mice transplanted with tumor-derived ΔS and ΔL cells. FIG. 7D: Survival curve of C57BL/6 mice transplanted with tumor-derived ΔS and AL cells. FIG. 7E: Representative images (left) and quantification (middle) of the fraction of Ki67+ cells in ΔS and ΔL tumors. (Right) Representative images of cleaved caspase-3 in in ΔS and ΔL tumors, which showed little to no detectable signal. FIG. 7F: Lung metastasis incidence in C57BL/6 mice with either ΔS or ΔL tumors. Bars represent fraction of metastasis-bearing mice (specific numbers of independently analyzed mice are noted in parentheses). ns=non-significant, chi-square test. FIG. 7G: Quantification of the number (left) and relative area (right) of liver and lung metastases in C57BL/6 mice with either AS or ΔL tumors. FIG. 7H: Metastasis incidence in C57BL/6 mice with either heterozygous or homozygous ΔL tumors. FIG. 7I: (Left) Metastasis incidence in C57BL/6 mice with AS, AI, or ΔL tumors. (Right) Copy number of Ifnb1, Ifne, Cdkn2a, and Cdkn2b in tumor-derived ΔI lines (Post) relative to pre-injection parental ΔI cells (Pre). Each dot represents an independent tumor-derived line. FIG. 7J: Macroscopic images of liver metastases in C57BL/6 mice after intrasplenic injection of either ΔS or ΔL cells. FIG. 7K: Relative area of liver metastases in C57BL/6 mice after intrasplenic injection of either ΔS or ΔL cells. FIG. 7L: Survival curve of Nude mice transplanted with tumor-derived ΔS and ΔL cells. FIG. 7M: Lung metastasis incidence in Nude mice with either ΔS or ΔL tumors. FIG. 7N: Analysis of 4C4 deletion status in PDAC GEMM cell lines derived from matched primary tumors (‘P’) and metastases (‘M’). sWGS was used to assess the status of the 4C4 locus. Deep blue color depicts deletion defined as log 2 relative abundance <−2.

FIG. 8A: Histogram of GSEA Normalized Enrichment Score (NES) highlighting the top 10 differentially expressed Hallmark gene datasets in ΔS and AL tumors. FIG. 8B: Heatmap of type I IFN response gene expression in ΔS and ΔL tumors. FIG. 8C: Heatmap of gene expression signatures for distinct immune subpopulations in AS and ΔL tumors. FIG. 8D: Relative mRNA expression of representative type I IFN genes (Ifnb1, Ifne) or type I IFN targets (Oasl1, Isg20), measured by RT-qPCR. Each dot represents an independent biological replicate. * p<0.05, ** p<0.01, *** p<0.001, two-tailed t-test. FIG. 8E: Experimental design for scRNA Seq analysis of CD45+ cells. CD45+ cells were sorted from three independent ΔS and ΔL tumors, uniquely labeled by antibody-coupled barcoding, pooled and processed for scRNA Seq analysis. FIG. 8F: Number of high-quality CD45+ cells recovered from ΔS and ΔL tumors. FIG. 8G: UMAP of library size per cell. FIG. 8H: Heatmap of genes used to identify specific subpopulations within CD45+ cells. FIG. 8I: Distribution of CD45+ cells across different subpopulations in ΔS and ΔL tumors. FIG. 8J: Average expression of the type I IFN response signature across antigen-presenting populations (B cells, dendritic cells, and macrophages) and CD8+ T cells. ***, p<0.001.

FIGS. 9A-9R: Immunophenotyping of infiltrating populations in ΔS and AL tumors. Frequency of CD45 cells (FIG. 9A), CD11b cells (FIG. 9B), CD3e cells (FIG. 9C), CD19 B cells (FIG. 9D), CD4 T cells (FIG. 9E), CD8 T cells and corresponding PD1 mean fluorescence intensity of CD44 CD8 T cells (FIG. 9F), tumor-associated macrophages (TAMs) including CD86+ and CD206+subtypes (FIG. 9G), CD11b and CD103 dendritic cell subsets (FIG. 9H), and myeloid-derived suppressor cells (MDSCs) including polymorphonuclear (PMN-MDSCs) and mononuclear (M-MDSCs) subtypes (FIG. 91). * p<0.05, ** p<0.01, *** p<0.001, ns=non-significant; two-tailed t-test. Each dot represents an independent biological replicate. FIG. 9J: UMAP of dendritic cell phenographs from ΔS or ΔL tumors. Known populations/states are circled. FIG. 9K: Frequency of dendritic cells across phenographs in ΔS or ΔL tumors. FIG. 9L: DAVID analysis of Gene Ontology Biological Processes enriched in AS-specific dendritic cells.

FIG. 9M: UMAP of macrophage phenographs from ΔS or ΔL tumors. Known populations/states are circled. FIG. 9N: Frequency of macrophages across phenographs in ΔS or ΔL tumors. FIG. 9O: DAVID analysis of Gene Ontology Biological Processes enriched in AS-specific macrophages. FIG. 9P: UMAP of B cell phenographs from ΔS or AL tumors. Known populations/states are circled. FIG. 9Q: Frequency of B cells across phenographs in ΔS or ΔL tumors. FIG. 9R: Enrichr analysis of the top Hallmark Pathways enriched in exhausted CD8+ T cells from ΔS and ΔL tumors.

FIG. 10A: GSEA enrichment scores (NES) of type I IFN signaling in mouse AS and human 9pS tumors compared to ΔL and 9 pL tumors, respectively. FIG. 10B: Comparison of GSEA NES scores for Reactome Pathways enriched in mouse ΔS (y axis) and human 9pS tumors (x axis). Highlighted are key pathways and immune populations enriched in IFN-proficient tumors. Circle size represents the adjusted p value. FIG. 10C: Comparison of GSEA NES scores and Immune populations enriched in mouse ΔS (y axis) and human 9pS tumors (x axis). Highlighted are key immune populations enriched in IFN-proficient tumors. Circle size represents the adjusted p value. FIG. 10D: GSEA enrichment scores (NES) of type I IFN signaling in human primary or metastatic 9pS tumors compared to 9 pL tumors from the COMPASS and TCGA datasets. FIG. 10E: Hallmark pathways downregulated in human PDAC liver metastases vs. primary tumors. Data from Moffitt et al., 201575.

FIG. 11A: Experimental outline to test the role of type I IFNAR signaling in transplantation experiments. FIG. 11B: Representative flow cytometry plots of EGFP fluorescence in ΔS or ΔL tumors from C57BL/6 mice treated with IgG or aIFNAR1 antibodies. FIG. 11C: Representative FACS plots of EGFP+ populations from IgG AL, IgG AS, or aIFNARI ΔS tumors. FIG. 11D: (Left) Representative bioluminescent images of primary tumors and intestines from mice with indicated genotypes of transplanted cells and antibody treatments. (Right) Quantification of all replicates. Boxes indicate the signal threshold for metastasis detection. * p<0.05, chi-square test. FIGS. 11E-11F: Representative H/E images (FIG. 11E) and quantification (FIG. 11F) of mesenteric lymph node metastases in mice with indicated genotypes of transplanted cells and antibody treatments. * p<0.05, two-tailed t-test comparing IgG vs IFNARI blockade in the corresponding cell lines. FIG. 11G: DAVID gene ontology analysis of a-IFNAR1 downregulated genes in ΔS tumors. Top 10 significant pathways are shown. FIG. 11H: IFNARI blockade specifically affects IFN signaling. NES scores of top 5 UP and DOWN Hallmark categories in tumors comparing ΔL vs ΔS (grey bars, data from FIG. 4C) or AL vs a-IFNARI ΔS (black bars). FIG. 111: RT-qPCR measurements of mRNA levels for Ifnb1 and Ifne in tumor cells and infiltrating CD45+ cells from ΔS and ΔL tumors. Dots represent independent tumors. FIG. 11J: qRT-PCR measurements of mRNA levels for Ifnb1 and Ifne in ΔS and ΔL tumor-derived cells after the indicated treatments. Dots represent independent cell lines.

FIG. 12A: Design of the vector for doxycycline-inducible expression of full-length mouse Ifne or a truncated version lacking the signal peptide as control. FIG. 12B: RT-qPCR of Ifne expression in cells cultured-/+doxycycline (2 mg/mL) for 72 hours. The assay specifically amplifies full-length Ifne. FIG. 12C: RT-qPCR of IFN target genes (Irf7, (asl1, Isg20) to in cells cultured-/+doxycycline (2 mg/mL) for 72 hours. FIG. 12D: Experimental design to test the role of sustained Ifne expression in immune competent and immune deficient mice. FIG. 12E: Survival curve of immune competent mice orthotopically transplanted with Ctrl or Ifne overexpressing ΔS and ΔL cells. n=5 per condition. * p<0.05; ** p<0.01, log rank test. FIG. 12F: Survival curve of immune deficient (nude) mice orthotopically transplanted with Ctrl or Ifne overexpressing ΔS and AL cells. n=5 per condition. n.s.=non-significant, log rank test. FIG. 12G: Representative image of an intestine from a mouse with sustained expression of Ctrl or full-length Ifne AL cells at endpoint. Arrowheads point to macrometastases in the mesentery and intestine. FIG. 12H: Incidence of overt liver metastasis in immune proficient and deficient hosts transplanted with ΔS or ΔL cells expressing Ctrl or full length Ifne (n=5). FIG. 12I: RT-qPCR of Ifne, Irf7, and Oasl1 in tumors from immune competent mice treated with doxycycline for 1 week before tumor analysis. Each dot represents an independent tumor (n=5). * p<0.05; *** p<0.001, one-way ANOVA followed by Sidak's multiple comparison test. FIG. 12J: Tumor immune infiltration of immune competent mice treated with doxycycline for 1 week before tumor analysis. Frequency of dendritic cells (far left), CD8 T cells (left), CD4 T cells (right), ad B cells (far right) are shown. Each dot represents an independent tumor (n=5). * p<0.05; ** p<0.01, n.s. =non-significant, one-way ANOVA followed by Sidak's multiple comparison test.

### DETAILED DESCRIPTION

It is to be appreciated that certain aspects, modes, embodiments, variations and features of the present methods are described below in various levels of detail in order to provide a substantial understanding of the present technology. It is to be understood that the present disclosure is not limited to particular uses, methods, reagents, compounds, compositions or biological systems, which can, of course, vary. It is also to be understood that the terminology used herein is for the purpose of describing particular embodiments only, and is not intended to be limiting.

In practicing the present methods, many conventional techniques in molecular biology, protein biochemistry, cell biology, immunology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology. Methods to detect and measure levels of polypeptide gene expression products (i.e., gene translation level) are well-known in the art and include the use of polypeptide detection methods such as antibody detection and quantification techniques. (See also, Strachan & Read, Human Molecular Genetics, Second Edition. (John Wiley and Sons, Inc., NY, 1999)).

The present disclosure demonstrates that cancer associated deletions that eliminate a cluster of 17 type I interferon genes and co-occur with well-known deletions of the cdkn2a gene cause developing tumor cells to evade immune surveillance and metastasize, and render the tumors resistant to checkpoint blockade. Importantly, the present disclosure demonstrates that restoring IFNE to tumor cells with deletions of the locus restores immune surveillance and suppresses metastasis. Without wishing to be bound by theory, it is believed that IFNE treatment of tumors with particular deletions (occur in ˜8% of all human tumors and 40% of pancreas cancer) are either directly therapeutic and/or restore sensitivity to checkpoint blockade.

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the “administration” of an agent or drug to a subject includes any route of introducing or delivering to a subject a compound to perform its intended function. Administration can be carried out by any suitable route, including but not limited to, orally, intranasally, parenterally (intravenously, intramuscularly, intraperitoneally, or subcutaneously), rectally, intrathecally, intratumorally or topically. Administration includes self-administration and the administration by another.

As used herein, a “bicistronic” vector or cassette refers to an expression vector or cassette consisting two distinct genes of interest within one vector or cassette. The vector transports the genes together into the cells, which simultaneously express both the genes of interest. A bicistronic vector can be made by using two-promoter systems, wherein the vector contains two separate expression cassettes with a different promoter for each gene. Two-promoter systems are ideal when both proteins are desired to be expressed at the same level because they tend to provide equal expression. However, the efficiency of expression can be affected by the size of the promoters and genes.

Alternatively, a bicistronic vector can be made by constructing a bicistronic cassette, wherein with a single promoter lead to simultaneously expression of two or more separate proteins from the same mRNA. Two strategies most widely used are described below.

IRES Elements. Translation in eukaryotes usually begins at the 5′ cap so that only a single translation event occurs for each mRNA. However, some bicistronic vectors take advantage of an element called an Internal Ribosome Entry Site (IRES) to allow for initiation of translation from an internal region of the mRNA. The IRES element acts as another ribosome recruitment site, thereby resulting in co-expression of two proteins from a single mRNA. IRES was originally discovered in poliovirus RNA, where it promotes translation of the viral genome in eukaryotic cells. Since then, a variety of IRES sequences have been discovered-many from viruses, but also some from cellular mRNAs. What they all have in common is the ability to spark translation initiation independent of the 5′ cap. IRES elements are very useful and commonly found in bicistronic vectors.

2A peptides. In some embodiments, “self-cleaving” 2A peptides have been adapted into bicistronic vectors. These peptides, first discovered in picornaviruses, are short (about 20 amino acids) and produce equimolar levels of multiple genes from the same mRNA. The term “self-cleaving” is not entirely accurate, as these peptides are thought to function by making the ribosome skip the synthesis of a peptide bond at the C-terminus of a 2A element, leading to separation between the end of the 2A sequence and the next peptide downstream. The “cleavage” occurs between the Glycine and Proline residues found on the C-terminus meaning the upstream cistron will have a few additional residues added to the end, while the downstream cistron will start with the Proline. Table 1 below lists the four commonly employed 2A peptides.

TABLE 1

| Peptide | Amino acid sequence* |
|---|---|
| T2A: | EGRGSLLTCGDVEENPGP (SEQ ID NO: 59) |
| P2A: | ATNFSLLKQAGDVEENPGP (SEQ ID NO: 60) |
| E2A: | QCTNYALLKLAGDVESNPGP (SEQ ID NO: 61) |
| F2A: | VKQTLNFDLLKLAGDVESNPGP (SEQ ID NO: 62) |

*(GSG) residues can be added to the 5′ end of the peptide to improve cleavage efficiency.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

As used herein, the term “engineer,” “engineering” or “engineered,” refers to genetic manipulation or modification of biomolecules such as DNA, RNA and/or protein, or like technique commonly known in the biotechnology art.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, the term “expression cassette” refers to a distinct component of a vector consisting of a gene and regulatory sequence to be expressed by a transfected cell. In each successful transformation, the expression cassette directs the cell's machinery to make RNA and protein(s). Expression cassettes basically consist of a promoter, the gene of interest (open reading frame, ORF), and a terminator.

As used herein, an “expression control sequence” refers to polynucleotide sequences which are necessary to affect the expression of coding sequences to which they are operably linked. Expression control sequences are sequences which control the transcription, post-transcriptional events and translation of nucleic acid sequences. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion. The nature of such control sequences differs depending upon the host organism; in prokaryotes, such control sequences generally include promoter, ribosomal binding site, and transcription termination sequence. The term “control sequences” is intended to encompass, at a minimum, any component whose presence is essential for expression, and can also encompass an additional component whose presence is advantageous, for example, leader sequences.

As used herein, “focal deletions” refer to regions of <5 million base pairs (Mb) with average log₂ ratios for neighboring probes of less than −2.

As used herein, the term “gene” means a segment of DNA that contains all the information for the regulated biosynthesis of an RNA product, including promoters, exons, introns, and other untranslated regions that control expression. When the gene encodes a protein, it includes the promoter and the structural gene open reading frame sequence (ORF), as well as other sequences involved in expression of the protein. When the gene encodes an untranslated RNA, it includes the promoter and the nucleic acid that encodes the untranslated RNA. A “native gene” or “endogenous gene” refers to a gene that is native to the host cell with its own regulatory sequences whereas an “exogenous gene” or “heterologous gene” refers to any gene that is not a native gene, comprising regulatory and/or coding sequences that are not native to the host cell. In some embodiments, an exogenous gene may comprise mutated sequences or part of regulatory and/or coding sequences. In some embodiments, the regulatory sequences may be heterologous or homologous to a gene of interest. A heterologous regulatory sequence does not function in nature to regulate the same gene(s) it is regulating in the transformed host cell.

As described herein, a “genetic component” or “genetic element” may be any coding or non-coding nucleic acid sequence. In some embodiments, a genetic component is a nucleic acid that codes for an amino acid, a peptide or a protein. Genetic components may be operons, genes, gene fragments, promoters, exons, introns, regulatory sequences, or any combination thereof. Genetic components can be as short as one or a few codons or may be longer including functional components (e.g., encoding proteins) and/or regulatory components. In some embodiments, a genetic component includes an entire open reading frame of a protein, or the entire open reading frame and one or more (or all) regulatory sequences associated therewith. One skilled in the art would appreciate that the genetic components can be viewed as modular genetic components or genetic element. For example, a genetic module can comprise a regulatory sequence or a promoter or a coding sequence or any combination thereof. In some embodiments, the genetic component includes at least two different genetic element and at least two recombination sites. In eukaryotes, the genetic component can comprise at least three modules. For example, a genetic module can be a regulator sequence or a promoter, a coding sequence, and a polyadenlylation tail or any combination thereof. In addition to the promoter and the coding sequences, the nucleic acid sequence may comprises control modules including, but not limited to a leader, a signal sequence and a transcription terminator. The leader sequence is a non-translated region operably linked to the 5′ terminus of the coding nucleic acid sequence. The signal peptide sequence codes for an amino acid sequence linked to the amino terminus of the polypeptide which directs the polypeptide into the cell's secretion pathway.

As generally understood, a codon is a series of three nucleotides (triplets) that encodes a specific amino acid residue in a polypeptide chain or for the termination of translation (stop codons). There are 64 different codons (61 codons encoding for amino acids plus 3 stop codons) but only 20 different translated amino acids. The overabundance in the number of codons allows many amino acids to be encoded by more than one codon. Different organisms (and organelles) often show particular preferences or biases for one of the several codons that encode the same amino acid. The relative frequency of codon usage thus varies depending on the organism and organelle. In some instances, when expressing a exogenous gene in a host organism, it is desirable to modify the gene sequence so as to adapt to the codons used and codon usage frequency in the host. In particular, for reliable expression of heterologous genes it may be preferred to use codons that correlate with the host's tRNA level, especially the tRNA's that remain charged during starvation. In addition, codons having rare cognate tRNA's may affect protein folding and translation rate, and thus, may also be used. Genes designed in accordance with codon usage bias and relative tRNA abundance of the host are often referred to as being “optimized” for codon usage, which has been shown to increase expression level. Optimal codons also help to achieve faster translation rates and high accuracy. In general, codon optimization involves silent mutations that do not result in a change to the amino acid sequence of a protein.

Genetic components or genetic element may derive from the genome of natural organisms or from synthetic polynucleotides or from a combination thereof. In some embodiments, the genetic components modules derive from different organisms. Genetic components or elements useful for the methods described herein may be obtained from a variety of sources such as, for example, DNA libraries, BAC (bacterial artificial chromosome) libraries, de novo chemical synthesis, commercial gene synthesis or excision and modification of a genomic segment. The sequences obtained from such sources may then be modified using standard molecular biology and/or recombinant DNA technology to produce polynucleotide constructs having desired modifications for reintroduction into, or construction of, a large product nucleic acid, including a modified, partially synthetic or fully synthetic genome. Exemplary methods for modification of polynucleotide sequences obtained from a genome or library include, for example, site directed mutagenesis; PCR mutagenesis; inserting, deleting or swapping portions of a sequence using restriction enzymes optionally in combination with ligation; in vitro or in vivo homologous recombination; and site-specific recombination; or various combinations thereof. In other embodiments, the genetic sequences useful in accordance with the methods described herein may be synthetic oligonucleotides or polynucleotides. Synthetic oligonucleotides or polynucleotides may be produced using a variety of methods known in the art.

As used herein, a “heterologous nucleic acid sequence” is any sequence placed at a location in the genome where it does not normally occur. A heterologous nucleic acid sequence may comprise a sequence that does not naturally occur in a particular cell, or it may comprise only sequences naturally found in the cell, but placed at a non-normally occurring location in the genome. In certain embodiments, the heterologous nucleic acid sequence is a synthetic sequence. In some embodiments, the heterologous nucleic acid sequence is a sequence from a donor cell that is biologically/phenotypically distinct from a recipient cell.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same nucleobase or amino acid, then the molecules are homologous at that position. A degree of homology between sequences is a function of the number of matching or homologous positions shared by the sequences. A polynucleotide or polynucleotide region (or a polypeptide or polypeptide region) has a certain percentage (for example, at least 60%, 65%, 70%, 75%, 80%, 85%, 90%), 95%), 98%) or 99%) of “sequence identity” to another sequence means that, when aligned, that percentage of bases (or amino acids) are the same in comparing the two sequences. This alignment and the percent homology or sequence identity can be determined using software programs known in the art. In some embodiments, default parameters are used for alignment. One alignment program is BLAST, using default parameters. In particular, programs are BLASTN and BLASTP, using the following default parameters: Genetic code=standard; filter=none; strand=both; cutoff=60; expect-10; Matrix=BLOSUM62, Descriptions=50 sequences; sort by =HIGH SCORE; Databases=non-redundant, GenBank+EMBL+DDBJ+PDB+GenBank CDStranslations+SwissProtein+SPupdate+PIR. Details of these programs can be found at the National Center for Biotechnology Information. Biologically equivalent polynucleotides are those having the specified percent homology and encoding a polypeptide having the same or similar biological activity Two sequences are deemed “unrelated” or “non-homologous” if they share less than 40% identity. or less than 25% identity, with each other.

As used herein, the phrase “homologous recombination” refers to the process in which nucleic acid molecules with similar nucleotide sequences associate and exchange nucleotide strands. A nucleotide sequence of a first nucleic acid molecule that is effective for engaging in homologous recombination at a predefined position of a second nucleic acid molecule can therefore have a nucleotide sequence that facilitates the exchange of nucleotide strands between the first nucleic acid molecule and a defined position of the second nucleic acid molecule. Thus, the first nucleic acid can generally have a nucleotide sequence that is sufficiently complementary to a portion of the second nucleic acid molecule to promote nucleotide base pairing. Homologous recombination requires homologous sequences in the two recombining partner nucleic acids but does not require any specific sequences. Homologous recombination can be used to introduce a heterologous nucleic acid and/or mutations into the host genome. Such systems typically rely on sequence flanking the heterologous nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

As used herein, “hybridization”, “hybridize”, or “hybridizing” refer to the binding of two complementary nucleotide sequences or substantially complementary sequences in which some mismatched base pairs may be present. The conditions for hybridization are well known in the art and vary based on the length of the nucleotide sequences and the degree of complementarity between the nucleotide sequences. In some embodiments, the conditions of hybridization can be high stringency, or they can be medium stringency or low stringency depending on the amount of complementarity and the length of the sequences to be hybridized. The conditions that constitute low, medium and high stringency for purposes of hybridization between nucleotide sequences are well known in the art (See, e.g., Gasiunas et al. (2012) Proc. Natl. Acad. Sci. 109: E2579-E2586; M. R. Green and J. Sambrook (2012) Molecular Cloning: A Laboratory Manual. 4th Ed., Cold Spring Harbor Laboratory Press, Cold Spring Harbor, N.Y.).

As used herein, the terms “individual”, “patient”, or “subject” can be an individual organism, a vertebrate, a mammal, or a human. In some embodiments, the individual, patient or subject is a human.

As used herein, the term “pharmaceutically-acceptable carrier” is intended to include any and all solvents, dispersion media, coatings, antibacterial and antifungal compounds, isotonic and absorption delaying compounds, and the like, compatible with pharmaceutical administration. Pharmaceutically-acceptable carriers and their formulations are known to one skilled in the art and are described, for example, in Remington's Pharmaceutical Sciences (20th edition, ed. A. Gennaro, 2000, Lippincott, Williams & Wilkins, Philadelphia, Pa.).

As used herein, the term “polynucleotide” or “nucleic acid” means any RNA or DNA, which may be unmodified or modified RNA or DNA. Polynucleotides include, without limitation, single- and double-stranded DNA, DNA that is a mixture of single- and double-stranded regions, single- and double-stranded RNA, RNA that is mixture of single- and double-stranded regions, and hybrid molecules comprising DNA and RNA that may be single-stranded or, more typically, double-stranded or a mixture of single- and double-stranded regions. In addition, polynucleotide refers to triple-stranded regions comprising RNA or DNA or both RNA and DNA. The term polynucleotide also includes DNAs or RNAs containing one or more modified bases and DNAs or RNAs with backbones modified for stability or for other reasons.

Nucleic acid sequences that are “complementary” are those that are capable of base-pairing according to the standard Watson-Crick complementarity rules. As used herein, the term “complementary sequences” may mean nucleic acid sequences that are 100% complementarity or less than 100% complementarity (e.g., about 80%, 81%, 82%, 83%, 84%, 85%, 86%, 87%, 88%, 89%, 90%, 91%, 92%, 93%, 94%, 95%, 96%, 97%, 98%, 99%, and the like, complementarity), or may be defined as being capable of hybridizing to the comparator polynucleotides.

As used herein, “prevention” or “preventing” of a disorder or condition refers to a compound that, in a statistical sample, reduces the occurrence of the disorder or condition in the treated sample relative to an untreated control sample, or delays the onset of one or more symptoms of the disorder or condition relative to the untreated control sample.

As used herein, the term “primer” refers to an oligonucleotide which is capable of annealing to the amplification target allowing a DNA polymerase to attach, thereby serving as a point of initiation of DNA synthesis when placed under conditions in which synthesis of primer extension product is induced, i.e., in the presence of nucleotides and an agent for polymerization such as DNA polymerase and at a suitable temperature and pH. The (amplification) primer is preferably single stranded for maximum efficiency in amplification. Preferably, the primer is an oligodeoxyribonucleotide. The primer must be sufficiently long to prime the synthesis of extension products in the presence of the agent for polymerization. The exact lengths of the primers will depend on many factors, including temperature and composition (A/T vs. G/C content) of primer. A pair of bi-directional primers consists of one forward and one reverse primer as commonly used in the art of DNA amplification such as in PCR amplification.

As used herein, the terms “promoter” or “promoter sequence” refer to a DNA sequence which when ligated to a nucleotide sequence of interest is capable of controlling the transcription of the nucleotide sequence of interest into mRNA or non-coding RNA. A promoter is typically, though not necessarily, located 5′ (i.e., upstream) of a nucleotide sequence of interest whose transcription into mRNA it controls, and provides a site for specific binding by RNA polymerase and other transcription factors for initiation of transcription. A promoter may be constitutively active (“constitutive promoter”) or be controlled by other factors such as a chemical, heat or light. The activity of an “inducible promoter” is induced by the presence or absence of biotic or abiotic factors. Commonly used constitutive promoters include CMV, EF1a, SV40, PGK1, Ubc, human beta actin, CAG, Ac5, Polyhedrin, TEF1, GDS, ADH1 (repressed by ethanol), CaMV35S, Ubi, H1, U6, T7 (requires T7 RNA polymerase), and SP6 (requires SP6 RNA polymerase). Common inducible promoters include TRE (inducible by Tetracycline or its derivatives; repressible by TetR repressor), GAL1 & GAL10 (inducible with galactose; repressible with glucose), lac (constitutive in the absence of lac repressor (LacI); can be induced by IPTG or lactose), T7lac (hybrid of T7 and lac; requires T7 RNA polymerase which is also controlled by lac operator; can be induced by TRIG or lactose), araBAD (inducible by arabinose which binds repressor AraC to switch it to activate transcription; repressed catabolite repression in the presence of glucose via the CAP binding site or by competitive binding of the anti-inducer fucose), trp (repressible by tryptophan upon binding with TrpR repressor), tac (hybrid of lac and trp; regulated like the lac promoter; e.g., tad and tacII), and pL (temperature regulated). The promoter can be prokaryotic or eukaryotic promoter, depending on the host. Common promoters and their sequences are well known in the art.

As used herein, the term “reporter” refers to a gene, operon, or protein that can be attached to a regulatory sequence of another gene or protein of interest, so that upon expression in a host cell or organism, the reporter can confer certain characteristics that can be relatively easily identified and/or measured. Reporter genes are often used as an indication of whether a certain gene has been introduced into or expressed in the host cell or organism. Examples of commonly used reporters include: antibiotic resistance genes, fluorescent proteins, auxotropic selection modules, β-galactosidase (encoded by the bacterial gene lacZ), luciferase (from lightning bugs), chloramphenicol acetyltransferase (CAT; from bacteria), GUS (B-glucuronidase; commonly used in plants) and green fluorescent protein (GFP; from jelly fish). Reporters or selection modules can be selectable or screenable.

As used herein, a “sample” or “biological sample” refers to a body fluid or a tissue sample isolated from a subject. In some cases, a biological sample may consist of or comprise whole blood, platelets, red blood cells, white blood cells, plasma, sera, urine, feces, epidermal sample, vaginal sample, skin sample, cheek swab, sperm, amniotic fluid, cultured cells, bone marrow sample, tumor biopsies, aspirate and/or chorionic villi, cultured cells, endothelial cells, synovial fluid, lymphatic fluid, ascites fluid, interstitial or extracellular fluid and the like. The term “sample” may also encompass the fluid in spaces between cells, including gingival crevicular fluid, bone marrow, cerebrospinal fluid (CSF), saliva, mucus, sputum, semen, sweat, urine, or any other bodily fluids. Samples can be obtained from a subject by any means including, but not limited to, venipuncture, excretion, ejaculation, massage, biopsy, needle aspirate, lavage, scraping, surgical incision, or intervention or other means known in the art. A blood sample can be whole blood or any fraction thereof, including blood cells (red blood cells, white blood cells or leukocytes, and platelets), serum and plasma.

As used herein, “selection marker” refers to a gene that confers a trait suitable for artificial selection. Selection marker genes can be categorized into negative selection marker and positive selection marker. Positive selection marker are used in positive selection systems, where only cells that contain the positive selection marker survive. Commonly used positive selection markers include antibiotic resistance marker and auxotrophic marker. Negative selection marker are used in negative selection systems, where only cells that have lost the negative selection marker survive. Negative selection markers are usually genes whose products are toxic. Commonly used negative selection markers include genes encoding toxin CcdB, levansucrase (SacB gene product), diphtheria toxin A (DT-A).

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

As used herein, the term “therapeutic agent” is intended to mean a compound that, when present in an effective amount, produces a desired therapeutic effect on a subject in need thereof.

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment or prevention of disorders as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

### CRISPR-Facilitated Homology-Directed Repair

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In some embodiments, a target sequence is located in the nucleus or cytoplasm of a cell. In some embodiments, the target sequence may be within an organelle of a eukaryotic cell, for example, mitochondrion or chloroplast. A sequence or template that may be used for recombination into the targeted locus comprising the target sequences is referred to as an “editing template” or “editing polynucleotide” or “editing sequence”. In aspects of the invention, an exogenous template polynucleotide may be referred to as an editing template. In an aspect of the invention the recombination is homologous recombination.

Typically, in the context of an endogenous CRISPR system, formation of a CRISPR complex (comprising a guide sequence hybridized to a target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. Without wishing to be bound by theory, the tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of a CRISPR complex, such as by hybridization along at least a portion of the tracr sequence to all or a portion of a tracr mate sequence that is operably linked to the guide sequence. In some embodiments, the tracr sequence has sufficient complementarity to a tracr mate sequence to hybridize and participate in formation of a CRISPR complex. As with the target sequence, it is believed that complete complementarity is not needed, provided there is sufficient to be functional. In some embodiments, the tracr sequence has at least 50%, 60%, 70%, 80%, 90%, 95% or 99% of sequence complementarity along the length of the tracr mate sequence when optimally aligned. In some embodiments, one or more vectors driving expression of one or more elements of a CRISPR system are introduced into a host cell such that expression of the elements of the CRISPR system direct formation of a CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In some embodiments, a single promoter drives expression of a transcript encoding a CRISPR enzyme and one or more of the guide sequence, tracr mate sequence (optionally operably linked to the guide sequence), and a tracr sequence embedded within one or more intron sequences (e.g. each in a different intron, two or more in at least one intron, or all in a single intron). In some embodiments, the CRISPR enzyme, guide sequence, tracr mate sequence, and tracr sequence are operably linked to and expressed from the same promoter.

In some embodiments, a vector comprises a regulatory element operably linked to an enzyme-coding sequence encoding a CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. In some embodiments, the CRISPR enzyme directs cleavage of one or both strands at the location of a target sequence, such as within the target sequence and/or within the complement of the target sequence. In some embodiments, the CRISPR enzyme directs cleavage of one or both strands within about 1, 2, 3, 4, 5, 6, 7, 8, 9, 10, 15, 20, 25, 50, 100, 200, 500, or more base pairs from the first or last nucleotide of a target sequence.

DNA double-stranded breaks can be repaired by, for example, non-homologous end joining (NHEJ) or homology-directed repair (HDR). As used herein, the term “homology-directed repair” or “HDR” refers to DNA repair that takes place in cells, for example, during repair of double-stranded and single-stranded breaks in DNA. HDR requires nucleotide sequence homology and uses a “donor template” (donor template DNA, polynucleotide donor, or oligonucleotide (used interchangeably herein) to repair the sequence where the double-stranded break occurred (e.g., DNA target sequence). This results in the transfer of genetic information from, for example, the donor template DNA to the DNA target sequence. HDR may result in alteration of the DNA target sequence (e.g., insertion, deletion, mutation) if the donor template DNA sequence or oligonucleotide sequence differs from the DNA target sequence and part or all of the donor template DNA polynucleotide or oligonucleotide is incorporated into the DNA target sequence. In some embodiments, an entire donor template DNA polynucleotide, a portion of the donor template DNA polynucleotide, or a copy of the donor polynucleotide is integrated at the site of the DNA target sequence.

### Bicistronic Expression Cassettes of the Present Technology and Uses Thereof

In one aspect, the present disclosure provides a donor nucleic acid template including a bicistronic expression cassette comprising a first cistron and a second cistron that are tandemly located, wherein the first cistron encodes a positive selection marker and the second cistron encodes a negative selection marker. The second cistron may be located at the 5′ end or the 3′ end of the first cistron. Additionally or alternatively, in some embodiments, a first artificial protospacer sequence is located upstream of the bicistronic expression cassette and/or a second artificial protospacer sequence is located at downstream of the bicistronic expression cassette. In certain embodiments, an Internal Ribosome Entry Site (IRES) sequence or a 2A peptide sequence is interspersed between the first cistron and the second cistron. The 2A peptide sequence may comprise any one of SEQ ID NOs: 59-62.

Additionally or alternatively, in some embodiments, the donor nucleic acid template further comprises a heterologous nucleic acid encoding an enzyme, a bioluminescent protein, a fluorescent protein, and/or a chemiluminescent protein, wherein the heterologous nucleic acid is located upstream or downstream of the bicistronic expression cassette. Fluorescent proteins include, but are not limited to, blue/UV fluorescent proteins (for example, TagBFP, Azurite, EBFP2, mKa1ama1, Sirius, Sapphire, and T-Sapphire), cyan fluorescent proteins (for example, ECFP, Cerulean, SCFP3A, m Turquoise, monomeric Midoriishi-Cyan, TagCFP, and mTFP1), green fluorescent proteins (for example, EGFP, Emerald, Superfolder GFP, Monomeric Azami Green, TagGFP2, mUKG, and mWasabi), yellow fluorescent proteins (for example, EYFP, Citrine, Venus, SYFP2, and TagYFP), orange fluorescent proteins (for example, Monomeric Kusabira-Orange, mKOK, mKO2, mOrange, and mOrange2), red fluorescent proteins (for example, mRaspberry, mCherry, dsRed, mStrawberry, mTangerine, tdTomato, TagRFP, TagRFP-T, mApple, and mRuby), far-red fluorescent proteins (for example, mPlum, HcRed-Tandem, mKate2, mNeptune, and NirFP), near-IR fluorescent proteins (for example, TagRFP657, IFP1.4, and iRFP), long stokes-shift proteins (for example, mKeima Red, LSS-mKate1, and LSS-mKate2), photoactivatable fluorescent proteins (for example, PA-GFP, PAmCherry 1, and PATagRFP), photoconvertible fluorescent proteins (for example, Kaede (green), Kaede (red), KikGR1 (green), KikGR1 (red), PS-CFP2, PS-CFP2, mEos2 (green), mEos2 (red), PSmOrange, and PSmOrange), fluorescein, rhodamine, and photoswitchable fluorescent proteins (for example, Dronpa). Examples of bioluminescent proteins are aequorin (derived from the jellyfish Aequorea victoria) and luciferases (including luciferases derived from firefly and Renilla, nanoluciferase, red luciferase, luxAB, and the like). Examples of chemiluminescent protein include β-galactosidase, horseradish peroxidase (HRP), and alkaline phosphatase.

Additionally or alternatively, in some embodiments, the bicistronic expression cassette is operably linked to an inducible promoter or a constitutive promoter. Examples of constitutive promoters include CMV, EF1a, SV40, PGK1, Ubc, human beta actin, CAG, Ac5, Polyhedrin, TEF1, GDS, ADH1 (repressed by ethanol), CaMV35S, Ubi, H1, U6, T7 (requires T7 RNA polymerase), and SP6 (requires SP6 RNA polymerase). Examples of inducible promoters include TRE (inducible by Tetracycline or its derivatives; repressible by TetR repressor), GAL1 & GAL10 (inducible with galactose; repressible with glucose), lac (constitutive in the absence of lac repressor (LacI); can be induced by IPTG or lactose), T7lac (hybrid of T7 and lac; requires T7 RNA polymerase which is also controlled by lac operator; can be induced by TRIG or lactose), araBAD (inducible by arabinose which binds repressor AraC to switch it to activate transcription; repressed catabolite repression in the presence of glucose via the CAP binding site or by competitive binding of the anti-inducer fucose), trp (repressible by tryptophan upon binding with TrpR repressor), tac (hybrid of lac and trp; regulated like the lac promoter; e.g., tad and tacII), and pL (temperature regulated).

Additionally or alternatively, in some embodiments, the positive selection marker is an antibiotic resistance gene. Examples of positive selection markers include, but are not limited to neomycin phosphotransferase, hygromycin phosphotransferase, phosphoinothricin acetyltransferase, glyphosate oxidoreductase, adenosine deaminase (ADA), aminoglycoside phosphotransferase, bleomycin, cytosine deaminase, dihydrofolate reductase, histidinol dehydrogenase, puromycin-N-acetyl transferase, thymidine kinase, or xanthine-guanine phosphoribosyltransferase.

Examples of negative selection markers include, but are not limited to herpes simplex virus thymidine kinase (HSV-TK), mlA, ypjF, ykfl, ydaS, yjhX, relE, mqsR, toxin CcdB, levansucrase, cytosine deaminase, or diphtheria toxin A (DT-A).

In one aspect, the present disclosure provides a method for knocking in a genetic alteration at a target gene locus in cells comprising: (a) contacting cells with a sgRNA-CRISPR enzyme conjugate in vivo under conditions where the sgRNA-CRISPR enzyme conjugate cleaves an endogenous protospacer sequence at the target gene locus in the cells to produce a cleaved target gene locus; (b) integrating the donor nucleic acid template of the present technology into the cleaved target gene locus via CRISPR-facilitated homology-directed repair, wherein the donor nucleic acid template comprises a 5′ flanking region and a 3′ flanking region that are homologous to the target gene locus; (c) enriching cells that stably express the positive selection marker; (d) contacting the enriched cells of step (c) with a first sgRNA-CRISPR enzyme complex and a second sgRNA-CRISPR enzyme complex in vivo under conditions where the first sgRNA-CRISPR enzyme complex cleaves the first artificial protospacer sequence within the donor nucleic acid template and the second sgRNA-CRISPR enzyme complex cleaves the second artificial protospacer sequence within the donor nucleic acid template to delete the bicistronic expression cassette; and (e) eliminating cells that stably express the negative selection marker to obtain a cell population comprising the genetic alteration at the target gene locus.

In another aspect, the present disclosure provides a method for knocking in a genetic alteration at a target gene locus in cells comprising: (a) contacting cells with a first sgRNA-CRISPR enzyme conjugate in vivo under conditions where the sgRNA-CRISPR enzyme conjugate cleaves a first endogenous protospacer sequence at the target gene locus in the cells to produce a cleaved target gene locus; (b) integrating the donor nucleic acid template of the present technology into the cleaved target gene locus via CRISPR-facilitated homology-directed repair, wherein the donor nucleic acid template comprises a 5′ flanking region and a 3′ flanking region that are homologous to the target gene locus; (c) enriching cells that stably express the positive selection marker; (d) contacting the enriched cells of step (c) with a second sgRNA-CRISPR enzyme complex and a third sgRNA-CRISPR enzyme complex in vivo under conditions where the second sgRNA-CRISPR enzyme complex cleaves the first artificial protospacer sequence within the donor nucleic acid template and the third sgRNA-CRISPR enzyme complex cleaves a second endogenous protospacer sequence at the target gene locus to delete the bicistronic expression cassette, wherein the second endogenous protospacer sequence is located downstream of the bicistronic expression cassette; and (e) eliminating cells that stably express the negative selection marker to obtain a cell population comprising the genetic alteration at the target gene locus.

Additionally or alternatively, in some embodiments of the methods disclosed herein, the homologous 5′ flanking region of the donor nucleic acid template has a length of about 20-30 base pairs (bps), 30-40 bps, 40-50 bps, 50-60 bps, 60-70 bps, 70-80 bps, 80-90 bps, 90-100 bps, 100-110 bps, 110-120 bps, 120-130 bps, 130-140 bps, 140-150 bps, 150-160 bps, 160-170 bps, 170-180 bps, 180-190 bps, 190-200 bps, 200-210 bps, 210-220 bps, 220-230 bps, 230-240 bps, 240-250 bps, 250-260 bps, 260-270 bps, 270-280 bps, 280-290 bps, 290-300 bps, 300-310 bps, 310-320 bps, 320-330 bps, 330-340 bps, 340-350 bps, 350-360 bps, 360-370 bps, 370-380 bps, 380-390 bps, 390-400 bps, 400-410 bps, 410-420 bps, 420-430 bps, 430-440 bps, 440-450 bps, 450-460 bps, 460-470 bps, 470-480 bps, 480-490 bps, 490-500 bps, 500-510 bps, 510-520 bps, 520-530 bps, 530-540 bps, 540-550 bps, 550-560 bps, 560-570 bps, 570-580 bps, 580-590 bps, or 590-600 bps.

Additionally or alternatively, in some embodiments of the methods disclosed herein, the homologous 3′ flanking region of the donor nucleic acid template sequence has a length of about 20-30 base pairs (bps), 30-40 bps, 40-50 bps, 50-60 bps, 60-70 bps, 70-80 bps, 80-90 bps, 90-100 bps, 100-110 bps, 110-120 bps, 120-130 bps, 130-140 bps, 140-150 bps, 150-160 bps, 160-170 bps, 170-180 bps, 180-190 bps, 190-200 bps, 200-210 bps, 210-220 bps, 220-230 bps, 230-240 bps, 240-250 bps, 250-260 bps, 260-270 bps, 270-280 bps, 280-290 bps, 290-300 bps, 300-310 bps, 310-320 bps, 320-330 bps, 330-340 bps, 340-350 bps, 350-360 bps, 360-370 bps, 370-380 bps, 380-390 bps, 390-400 bps, 400-410 bps, 410-420 bps, 420-430 bps, 430-440 bps, 440-450 bps, 450-460 bps, 460-470 bps, 470-480 bps, 480-490 bps, 490-500 bps, 500-510 bps, 510-520 bps, 520-530 bps, 530-540 bps, 540-550 bps, 550-560 bps, 560-570 bps, 570-580 bps, 580-590 bps, or 590-600 bps.

In any and all embodiments of the methods disclosed herein, the cells are mammalian cells, embryonic stem cells, or fibroblasts.

### Interferon Epsilon (IFNE)

The present disclosure demonstrates that interferon E (IFNE) treatment of tumor cells with Cdkn2a and Cdkn2b deletions restores immune surveillance, suppresses metastasis, and restores sensitivity to immune checkpoint blockade therapy. In some embodiments, the IFNE amino acid sequence may be human, primate, murine, bovine, ovine, canine, feline etc.

An exemplary nucleic acid sequence of human IFNE is set forth in SEQ ID NO: 63, as provided below:

>NM 176891.4 Homo sapiens interferon epsilon (IFNE), mRNA
(SEQ ID NO: 63)
CTTAGATATTAAACTGATAGGATAAGATATAAAATAATTTAAGATTGCTGATATATGTTTTAAAATTAAT
TATTTGCTCAAGCATTTGTGACAATTTACAGTTCTAATTGAGGTTTTAAATTTAGTAGTTTGTAGGTATT
TTAAGTTTTGCCCCTGAATTCTTTATAGGTGCTGATAAGCCTTTGGTAAGTTTTACTCCATGAAAGACTA
TTACTGAAAAAAACGTAATCTCAATAAAAGAACTTTAATAAGCTTGACTAAATATTTAGAAAGCACATTG
TGTTCAGTGAAACTTTGTATATAATGAATAGAATAATAAAAGATTATGTTGGATGACTAGTCTGTAATTG
CCTCAAGGAAAGCATACAATGAATAAGTTATTTTGGTACTTCCTCAAAATAGCCAACACAATAGGGAAAT
GGAGAAAATGTACTCTGAACACCATGAAAAGGGAACCTGAAAATCTAATGTGTAAACTTGGAGAAATGAC
ATTAGAAAACGAAAGCAACAAAAGAGAACACTCTCCAAAATAATCTGAGATGCATGAAAGGCAAACATTC
ACTAGAGCTGGAATTTCCCTAAGTCTATGCAGGGATAAGTAGCATATTTGACCTTCACCATGATTATCAA
GCACTTCTTTGGAACTGTGTTGGTGCTGCTGGCCTCTACCACTATCTTCTCTCTAGATTTGAAACTGATT
ATCTTCCAGCAAAGACAAGTGAATCAAGAAAGTTTAAAACTCTTGAATAAGTTGCAAACCTTGTCAATTC
AGCAGTGTCTACCACACAGGAAAAACTTTCTGCTTCCTCAGAAGTCTTTGAGTCCTCAGCAGTACCAAAA
AGGACACACTCTGGCCATTCTCCATGAGATGCTTCAGCAGATCTTCAGCCTCTTCAGGGCAAATATTTCT
CTGGATGGTTGGGAGGAAAACCACACGGAGAAATTCCTCATTCAACTTCATCAACAGCTAGAATACCTAG
AAGCACTCATGGGACTGGAAGCAGAGAAGCTAAGTGGTACTTTGGGTAGTGATAACCTTAGATTACAAGT
TAAAATGTACTTCCGAAGGATCCATGATTACCTGGAAAACCAGGACTACAGCACCTGTGCCTGGGCCATT
GTCCAAGTAGAAATCAGCCGATGTCTGTTCTTTGTGTTCAGTCTCACAGAAAAACTGAGCAAACAAGGAA
GACCCTTGAACGACATGAAGCAAGAGCTTACTACAGAGTTTAGAAGCCCGAGGTAGGTGGAGGGACTAGA
GGACTTCTCCAGACATGATTCTTCATAGAGTGGTAATACAATTTATAGTACAATCACATTGCTTTGATTT
TGTGTATATATATATTTATCTGTGTTTTAAGATTGTGCATATTGACCACAATTGTTTTTATTTTGTAATG
TGGCTTTATATATTCTATCCATTTTAAATTGTTTGTATGTCAAAATAAATTCATTAATATGGTTGATTCT
TCAAAAAAAAAAAAAAAAAAAAAAAAAAAAA

In some embodiments, the IFNE nucleic acid sequence is at least 80%, 85%, 90%, 95%, 96%, 97%, 98%, or 99% identical to SEQ ID NO: 63.

An exemplary amino acid sequence of human IFNE is set forth in SEQ ID NO: 64, as provided below:

(SEQ ID NO: 64)
MIIKHFFGTVLVLLASTTIFSLDLKLIIFQQRQVNQESLKLLNKLQTLS
IQQCLPHRKNFLLPQKSLSPQQYQKGHTLAILHEMLQQIFSLFRANISL
DGWEENHTEKFLIQLHQQLEYLEALMGLEAEKLSGTLGSDNLRLQVKMY
FRRIHDYLENQDYSTCAWAIVQVEISRCLFFVFSLTEKLSKQGRPLNDM
KQELTTEFRSPR

In some embodiments, the IFNE amino acid sequence is at least 80%, 85%, 90%, 95%, 96%, 97%, 98%, or 99% identical to SEQ ID NO: 64.

Also included in the presently disclosed subject matter are IFNE polynucleotides and their corresponding polypeptides or fragments that may be modified in ways that enhance their anti-tumor activity when administered to a patient in need thereof. The presently disclosed subject matter provides methods for optimizing an amino acid sequence or a nucleic acid sequence by producing an alteration in the sequence. Such alterations can comprise certain mutations, deletions, insertions, or post-translational modifications. The presently disclosed subject matter further comprises analogs of any naturally-occurring polypeptide of the presently disclosed subject matter. Analogs can differ from a naturally-occurring polypeptide of the presently disclosed subject matter by amino acid sequence differences, by post-translational modifications, or by both. Analogs of the presently disclosed subject matter can generally exhibit at least about 85%, about 90%, about 91%, about 92%, about 93%, about 94%, about 95%, about 96%, about 97%), about 98%, about 99% or more identity or homology with all or part of a naturally-occurring amino, acid sequence of the presently disclosed subject matter. The length of sequence comparison is at least about 5, about 10, about 15, about 20, about 25, about 50, about 75, about 100 or more amino acid residues. Again, in an exemplary approach to determining the degree of identity, a BLAST program can be used, with a probability score between e⁻³ and e⁻¹⁰⁰ indicating a closely related sequence. Modifications comprise in vivo and in vitro chemical derivatization of polypeptides, e.g., acetylation, carboxylation, phosphorylation, or glycosylation; such modifications can occur during polypeptide synthesis or processing or following treatment with isolated modifying enzymes. Analogs can also differ from the naturally-occurring polypeptides of the presently disclosed subject matter by alterations in primary sequence. These include genetic variants, both natural and induced (for example, resulting from random mutagenesis by irradiation or exposure to ethanemethyl sulfate or by site-specific mutagenesis as described in Sambrook, Fritsch and Maniatis, Molecular Cloning: A Laboratory Manual (2nd ed.), CSH Press, 1989, or Ausubel et al., supra). Also included are cyclized peptides, molecules, and analogs which contain residues other than L-amino acids, e.g., D-amino acids or non-naturally occurring or synthetic amino acids, e.g., beta (β) or gamma (γ) amino acids.

In addition to full-length polypeptides, the presently disclosed subject matter also provides fragments of any one of the polypeptides or peptide domains of the presently disclosed subject matter. A fragment can be at least about 5, about 10, about 13, or about 15 amino acids. In some embodiments, a fragment is at least about 20 contiguous amino acids, at least about 30 contiguous amino acids, or at least about 50 contiguous amino acids. In some embodiments, a fragment is at least about 60 to about 80, about 100, about 200, about 300 or more contiguous amino acids. Fragments of the presently disclosed subject matter can be generated by methods known to those of ordinary skill in the art or can result from normal protein processing (e.g., removal of amino acids from the nascent polypeptide that are not required for biological activity or removal of amino acids by alternative mRNA splicing or alternative protein processing events).

Non-protein analogs have a chemical structure designed to mimic the functional activity of a protein. Such analogs are administered according to methods of the presently disclosed subject matter. Such analogs can exceed the physiological activity of the original polypeptide. Methods of analog design are well known in the art, and synthesis of analogs can be carried out according to such methods by modifying the chemical structures such that the resultant analogs increase the antineoplastic activity of the original polypeptide when expressed in a cancer patient in need thereof. These chemical modifications include, but are not limited to, substituting alternative R groups and varying the degree of saturation at specific carbon atoms of a reference polypeptide. The protein analogs can be relatively resistant to in vivo degradation, resulting in a more prolonged therapeutic effect upon administration. Assays for measuring functional activity include, but are not limited to, those described in the Examples below.

In accordance with the presently disclosed subject matter, the polynucleotides encoding IFNE can be modified by codon optimization. Codon optimization can alter both naturally occurring and recombinant gene sequences to achieve the highest possible levels of productivity in any given expression system. Factors that are involved in different stages of protein expression include codon adaptability, mRNA structure, and various cis-elements in transcription and translation. Any suitable codon optimization methods or technologies that are known to ones skilled in the art can be used to modify the polynucleotides of the presently disclosed subject matter, including, but not limited to, OptimumGene™, Encor optimization, and Blue Heron.

### Formulations Including IFNE of the Present Technology

In one aspect, the present disclosure provides pharmaceutical compositions comprising Interferon Epsilon (IFNE).

The pharmaceutical compositions of the present disclosure may be prepared by any of the methods known in the pharmaceutical arts. The amount of active ingredient that can be combined with a carrier material to produce a single dosage form will vary depending upon the host being treated and the particular mode of administration. The amount of active ingredient that can be combined with a carrier material to produce a single dosage form will generally be that amount of the compound that produces a therapeutic effect. Generally, the amount of active compound will be in the range of about 0.1 to 99 percent, more typically, about 5 to 70 percent, and more typically, about 10 to 30 percent.

In some embodiments, pharmaceutical compositions of the present technology may contain one or more pharmaceutically-acceptable carriers, which as used herein, generally refers to a pharmaceutically-acceptable composition, such as a liquid or solid filler, diluent, excipient, manufacturing aid (e.g., lubricant, talc magnesium, calcium or zinc stearate, or steric acid), or solvent encapsulating material, useful for introducing the active agent into the body.

Examples of suitable aqueous and non-aqueous carriers that may be employed in the pharmaceutical compositions of the present technology include, for example, water, ethanol, polyols (such as glycerol, propylene glycol, polyethylene glycol, and the like), vegetable oils (such as olive oil), and injectable organic esters (such as ethyl oleate), and suitable mixtures thereof. Proper fluidity can be maintained, for example, by the use of coating materials, such as lecithin, by the maintenance of the required particle size in the case of dispersions, and by the use of surfactants.

In some embodiments, the formulations may include one or more of sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; alginic acid; buffering agents, such as magnesium hydroxide and aluminum hydroxide; pyrogen-free water; isotonic saline; Ringer's solution; ethyl alcohol; pH buffered solutions; polyesters, polycarbonates and/or polyanhydrides; preservatives; glidants; fillers; and other non-toxic compatible substances employed in pharmaceutical formulations.

Various auxiliary agents, such as wetting agents, emulsifiers, lubricants (e.g., sodium lauryl sulfate and magnesium stearate), coloring agents, release agents, coating agents, sweetening agents, flavoring agents, preservative agents, and antioxidants can also be included in the pharmaceutical composition of the present technology. Some examples of pharmaceutically-acceptable antioxidants include: water soluble antioxidants, such as ascorbic acid, cysteine hydrochloride, sodium bisulfate, sodium metabisulfite, sodium sulfite, and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like. In some embodiments, the pharmaceutical formulation includes an excipient selected from, for example, celluloses, liposomes, lipid nanoparticles, micelle-forming agents (e.g., bile acids), and polymeric carriers, e.g., polyesters and polyanhydrides. Suspensions, in addition to the active compounds, may contain suspending agents, such as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof. Prevention of the action of microorganisms on the active compounds may be ensured by the inclusion of various antibacterial and antifungal agents, such as, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption, such as aluminum monostearate and gelatin.

The pharmaceutical compositions of the present technology can be manufactured by methods well known in the art such as conventional granulating, mixing, dissolving, encapsulating, lyophilizing, or emulsifying processes, among others. Compositions may be produced in various forms, including granules, precipitates, or particulates, powders, including freeze dried, rotary dried or spray dried powders, amorphous powders, tablets, capsules, syrup, suppositories, injections, emulsions, elixirs, suspensions or solutions. Formulations may optionally contain solvents, diluents, and other liquid vehicles, dispersion or suspension aids, surface active agents, pH modifiers, isotonic agents, thickening or emulsifying agents, stabilizers and preservatives, solid binders, lubricants and the like, as suited to the particular dosage form desired. In certain embodiments, the compositions disclosed herein are formulated for administration to a mammal, such as a human.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, cyclodextrins, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. Compositions formulated for parenteral administration may be injected by bolus injection or by timed push, or may be administered by continuous infusion.

In order to prolong the effect of a compound of the present disclosure, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents such as phosphates or carbonates.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings, release controlling coatings and other coatings well known in the pharmaceutical formulating art. They may optionally contain opacifying agents and can also be of a composition that they release the active ingredient(s) only, or in a certain part of the intestinal tract, optionally, in a delayed manner. Examples of embedding compositions that can be used include polymeric substances and waxes.

The active compounds can also be in micro-encapsulated form with one or more excipients as noted above. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents.

### Modes of Administration and Effective Dosages

Any method known to those in the art for contacting a cell, organ or tissue with one or more compositions comprising IFNE disclosed herein may be employed. Suitable methods include in vitro, ex vivo, or in vivo methods. In vivo methods typically include the administration of one or more compositions comprising IFNE to a mammal, suitably a human. When used in vivo for therapy, the one or more compositions comprising IFNE described herein are administered to the subject in effective amounts (i.e., amounts that have desired therapeutic effect). The dose and dosage regimen will depend upon the degree of the disease state of the subject, the characteristics of the therapeutic agent used, e.g., its therapeutic index, and the subject's history.

The effective amount may be determined during pre-clinical trials and clinical trials by methods familiar to physicians and clinicians. An effective amount of one or more compositions comprising IFNE useful in the methods may be administered to a mammal in need thereof by any of a number of well-known methods for administering pharmaceutical compounds. The one or more compositions comprising IFNE may be administered systemically or locally.

The one or more compositions comprising IFNE described herein can be incorporated into pharmaceutical compositions for administration, singly or in combination, to a subject for the treatment or prevention of cancer in a patient in need thereof, wherein the patient comprises focal deletions in Cdkn2a and Cdkn2b. Such compositions typically include the active agent and a pharmaceutically acceptable carrier. As used herein the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions are typically formulated to be compatible with its intended route of administration. Examples of routes of administration include parenteral (e.g., intravenous, intradermal, intraperitoneal or subcutaneous), oral, inhalation, transdermal (topical), intraocular, iontophoretic, and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. For convenience of the patient or treating physician, the dosing formulation can be provided in a kit containing all necessary equipment (e.g., vials of drug, vials of diluent, syringes and needles) for a treatment course (e.g., 7 days of treatment).

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, CREMOPHOR EL™ (BASF, Parsippany, N.J.) or phosphate buffered saline (PBS). In all cases, a composition for parenteral administration must be sterile and should be fluid to the extent that easy syringability exists. It should be stable under the conditions of manufacture and storage and must be preserved against the contaminating action of microorganisms such as bacteria and fungi.

The pharmaceutical compositions comprising IFNE disclosed herein can include a carrier, which can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thiomerasol, and the like. Glutathione and other antioxidants can be included to prevent oxidation. In many cases, it will be advantageous to include isotonic agents, for example, sugars, polyalcohols such as mannitol, sorbitol, or sodium chloride in the composition. Prolonged absorption of the injectable compositions can be brought about by including in the composition an agent that delays absorption, for example, aluminum monostearate or gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressurized container or dispenser, which contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays. For transdermal administration, the active compounds are formulated into ointments, salves, gels, or creams as generally known in the art. In one embodiment, transdermal administration may be performed by iontophoresis.

A therapeutic agent can be formulated in a carrier system. The carrier can be a colloidal system. The colloidal system can be a liposome, a phospholipid bilayer vehicle, or a lipid nanoparticle. In one embodiment, the therapeutic agent is encapsulated in a liposome while maintaining the agent's structural integrity. One skilled in the art would appreciate that there are a variety of methods to prepare liposomes. (See Lichtenberg, et al., Methods Biochem. Anal., 33:337-462 (1988); Anselem, et al., Liposome Technology, CRC Press (1993)). Liposomal formulations can delay clearance and increase cellular uptake (See Reddy, Ann. Pharmacother., 34 (7-8): 915-923 (2000)). An active agent can also be loaded into a particle prepared from pharmaceutically acceptable ingredients including, but not limited to, soluble, insoluble, permeable, impermeable, biodegradable or gastroretentive polymers or liposomes. Such particles include, but are not limited to, nanoparticles, biodegradable nanoparticles, microparticles, biodegradable microparticles, nanospheres, biodegradable nanospheres, microspheres, biodegradable microspheres, capsules, emulsions, liposomes, micelles and viral vector systems.

The carrier can also be a polymer, e.g., a biodegradable, biocompatible polymer matrix. In one embodiment, the therapeutic agent can be embedded in the polymer matrix, while maintaining the agent's structural integrity. The polymer may be natural, such as polypeptides, proteins or polysaccharides, or synthetic, such as poly a-hydroxy acids. Examples include carriers made of, e.g., collagen, fibronectin, elastin, cellulose acetate, cellulose nitrate, polysaccharide, fibrin, gelatin, and combinations thereof. In one embodiment, the polymer is poly-lactic acid (PLA) or copoly lactic/glycolic acid (PGLA). The polymeric matrices can be prepared and isolated in a variety of forms and sizes, including microspheres and nanospheres. Polymer formulations can lead to prolonged duration of therapeutic effect. (See Reddy, Ann. Pharmacother., 34 (7-8): 915-923 (2000)). A polymer formulation for human growth hormone (hGH) has been used in clinical trials. (See Kozarich and Rich, Chemical Biology, 2:548-552 (1998)).

Examples of polymer microsphere sustained release formulations are described in PCT publication WO 99/15154 (Tracy, et al.), U.S. Pat. Nos. 5,674,534 and 5,716,644 (both to Zale, et al.), PCT publication WO 96/40073 (Zale, et al.), and PCT publication WO 00/38651 (Shah, et al.). U.S. Pat. Nos. 5,674,534 and 5,716,644 and PCT publication WO 96/40073 describe a polymeric matrix containing particles of erythropoietin that are stabilized against aggregation with a salt.

In some embodiments, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using known techniques. The materials can also be obtained commercially, e.g., from Alza Corporation and Nova Pharmaceuticals, Inc. Liposomal suspensions (including liposomes targeted to specific cells with monoclonal antibodies to cell-specific antigens) can also be used as pharmaceutically acceptable carriers. These can be prepared according to methods known to those skilled in the art, for example, as described in U.S. Pat. No. 4,522,811.

The therapeutic compounds can also be formulated to enhance intracellular delivery. For example, liposomal delivery systems are known in the art, see, e.g., Chonn and Cullis, “Recent Advances in Liposome Drug Delivery Systems,” Current Opinion in Biotechnology 6:698-708 (1995); Weiner, “Liposomes for Protein Delivery: Selecting Manufacture and Development Processes,” Immunomethods, 4 (3): 201-9 (1994); and Gregoriadis, “Engineering Liposomes for Drug Delivery: Progress and Problems,” Trends Biotechnol., 13 (12): 527-37 (1995). Mizguchi, et al., Cancer Lett., 100:63-69 (1996), describes the use of fusogenic liposomes to deliver a protein to cells both in vivo and in vitro.

Dosage, toxicity and therapeutic efficacy of any therapeutic agent can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are advantageous. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds may be within a range of circulating concentrations that include the ED50 with little or no toxicity. The dosage may vary within this range depending upon the dosage form employed and the route of administration utilized. For any compound used in the methods, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to determine useful doses in humans accurately. Levels in plasma may be measured, for example, by high performance liquid chromatography.

Typically, an effective amount of the one or more compositions comprising IFNE disclosed herein sufficient for achieving a therapeutic or prophylactic effect, range from about 0.000001 mg per kilogram body weight per day to about 10,000 mg per kilogram body weight per day. Suitably, the dosage ranges are from about 0.0001 mg per kilogram body weight per day to about 100 mg per kilogram body weight per day. For example, dosages can be 1 mg/kg body weight or 10 mg/kg body weight every day, every two days or every three days or within the range of 1-10 mg/kg every week, every two weeks or every three weeks. In one embodiment, a single dosage of the therapeutic compound ranges from 0.001-10,000 micrograms per kg body weight. In one embodiment, IFNE concentrations in a carrier range from 0.2 to 2000 micrograms per delivered milliliter. An exemplary treatment regime entails administration once per day or once a week. In therapeutic applications, a relatively high dosage at relatively short intervals is sometimes required until progression of the disease is reduced or terminated, or until the subject shows partial or complete amelioration of symptoms of disease. Thereafter, the patient can be administered a prophylactic regime.

In some embodiments, a therapeutically effective amount of one or more compositions comprising IFNE may be defined as a concentration of inhibitor at the target tissue of 10⁻³² to 10⁻⁶ molar, e.g., approximately 10⁻⁷ molar. This concentration may be delivered by systemic doses of 0.001 to 100 mg/kg or equivalent dose by body surface area. The schedule of doses would be optimized to maintain the therapeutic concentration at the target tissue, such as by single daily or weekly administration, but also including continuous administration (e.g., parenteral infusion or transdermal application).

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

The mammal treated in accordance with the present methods can be any mammal, including, for example, farm animals, such as sheep, pigs, cows, and horses; pet animals, such as dogs and cats; laboratory animals, such as rats, mice and rabbits. In some embodiments, the mammal is a human.

### Therapeutic Methods of the Present Technology

In one aspect, the present disclosure provides a method for treating cancer in a patient in need thereof comprising administering to the patient an effective amount of IFNE, wherein the patient comprises focal deletions in Cdkn2a and Cdkn2b. Also provided herein is a method for enhancing responsiveness to immune checkpoint blockade therapy in a cancer patient in need thereof comprising administering to the patient an effective amount of IFNE and an effective amount of an immune checkpoint inhibitor, wherein the patient comprises focal deletions in Cdkn2a and Cdkn2b. In some embodiments, the immune checkpoint inhibitor is an anti-PD-1 antibody, an anti-PD-L1 antibody, an anti-PD-L2 antibody, an anti-CTLA-4 antibody, an anti-TIM3 antibody, an anti-4-1BB antibody, an anti-CD73 antibody, an anti-GITR antibody, or an anti-LAG-3 antibody. Additionally or alternatively, in some embodiments, the immune checkpoint inhibitor may be selected from among CTLA4 (for example, Yervoy (ipilimumab), CP-675,206 (tremelimumab), AK104 (cadonilimab), or AGEN1884 (zalifrelimab)), or an antibody or an equivalent thereof recognizing and binding to PD-1 (for example, Keytruda (pembrolizumab), Opdivo (nivolumab), Libtayo (cemiplimab), Tyvyt (sintilimab), BGB-A317 (tislelizumab), JS001 (toripalimab), SHR1210 (camrelizumab), GB226 (geptanolimab), JS001 (toripalimab), AB122 (zimberelimab), AK105 (penpulimab), HLX10 (serplulimab), BCD-100 (prolgolimab), AGEN2034 (balstilimab), MGA012 (retifanlimab), AK104 (cadonilimab), HX008 (pucotenlimab), PF-06801591 (sasanlimab), JNJ-63723283 (cetrelimab), MGD013 (tebotelimab), CT-011 (pidilizumab), or Jemperli (dostarlimab)), or an antibody or an equivalent thereof recognizing and binding to PD-L1 (for example, Tecentriq (atezolizumab), Imfinzi (durvalumab), Bavencio (avelumab), CS1001 (sugemalimab), or KN035 (envafolimab)).

Additionally or alternatively, in some embodiments, the focal deletions in Cdkn2a and Cdkn2b are no more than 0.4 Mb, no more than 0.3 Mb, no more than 0.2 Mb, no more than 0.1 Mb, no more than 90 Kb, no more than 80 Kb, no more than 70 Kb, no more than 60 Kb, no more than 50 Kb, no more than 40 Kb, no more than 30 Kb, no more than 20 Kb, no more than 10 Kb, no more than 9 Kb, no more than 8 Kb, no more than 7 Kb, no more than 6 Kb, no more than 5 Kb, no more than 4 Kb, no more than 3 Kb, no more than 2 Kb, or no more than 1 Kb in length.

In some embodiments of the methods disclosed herein, the patient further comprises deletions in at least one IFN gene in type I IFN cluster. The type I IFN cluster may comprise IFN-α1, IFN-α2, IFN-α4, IFN-α5, IFN-α6, IFN-α7, IFN-α8, IFN-α10, IFN-α13, IFN-α14, IFN-α16, IFN-α17, IFN-α21, IFNB, IFN-Epsilon, IFN-Kappa, and IFN-Omega. In certain embodiments, deletions in the type I IFN cluster are no more than 1.3 Mb, no more than 1.2 Mb, no more than 1.1 Mb, no more than 1 Mb, no more than 0.9 Mb, no more than 0.8 Mb, no more than 0.7 Mb, no more than 0.6 Mb, no more than 0.5 Mb, or no more than 0.4 Mb in length.

In any and all embodiments of the methods disclosed herein, the cancer may be selected from among lung cancer, pancreatic cancer, head and neck squamous cell cancer, esophageal carcinoma, skin cutaneous melanoma, stomach cancer, glioblastoma, bladder urothelial carcinoma, or brain lower grade glioma. In some embodiments, the pancreatic cancer is pancreatic adenocarcinoma (PDAC). In other embodiments, the lung cancer is lung adenocarcinoma (LUAD) or lung squamous cell carcinoma.

In any and all embodiments of the methods disclosed herein, the IFNE is administered orally, topically, intranasally, systemically, intravenously, subcutaneously, intraperitoneally, intradermally, intraocularly, iontophoretically, transmucosally, or intramuscularly. Additionally or alternatively, in some embodiments, the IFNE comprises, consists essentially of, or consists of an amino acid sequence that is at least 80%, 85%, 90%, 95%, 96%, 97%, 98%, or 99% identical to SEQ ID NO: 64.

### Kits

The present disclosure also provides kits for the prevention and/or treatment of a cancer (e.g., pancreatic cancer) in a patient, comprising one or more compositions including IFNE. Optionally, the above described components of the kits of the present technology are packed in suitable containers and labeled for the prevention and/or treatment of cancer (e.g., pancreatic cancer). In some embodiments, the patient comprises focal deletions in Cdkn2a and Cdkn2b.

The above-mentioned components may be stored in unit or multi-dose containers, for example, sealed ampoules, vials, bottles, syringes, and test tubes, as an aqueous, preferably sterile, solution or as a lyophilized, preferably sterile, formulation for reconstitution. The kit may further comprise a second container which holds a diluent suitable for diluting the pharmaceutical composition towards a higher volume. Suitable diluents include, but are not limited to, the pharmaceutically acceptable excipient of the pharmaceutical composition and a saline solution. Furthermore, the kit may comprise instructions for diluting the pharmaceutical composition and/or instructions for administering the pharmaceutical composition, whether diluted or not. The containers may be formed from a variety of materials such as glass or plastic and may have a sterile access port (for example, the container may be an intravenous solution bag or a vial having a stopper which may be pierced by a hypodermic injection needle). The kit may further comprise more containers comprising a pharmaceutically acceptable buffer, such as phosphate-buffered saline, Ringer's solution and dextrose solution. It may further include other materials desirable from a commercial and user standpoint, including other buffers, diluents, filters, needles, syringes, culture medium for one or more of the suitable hosts. The kits may optionally include instructions customarily included in commercial packages of therapeutic products, that contain information about, for example, the indications, usage, dosage, manufacture, administration, contraindications and/or warnings concerning the use of such therapeutic products.

The kit can also comprise, e.g., a buffering agent, a preservative or a stabilizing agent. The kits of the present technology may contain a written product on or in the kit container. The written product describes how to use the reagents contained in the kit. In certain embodiments, the use of the reagents can be according to the methods of the present technology.

### Examples

The present technology is further illustrated by the following Examples, which should not be construed as limiting in any way.

### Example 1: Materials and Methods

Pan-cancer TCGA Data Analysis. Analysis of TCGA datasets was performed on cBioPortal^{63,64}. All TCGA datasets were selected and the following onco-query language (OQL) entry was used (for 9p21.3 OQL). Tumors with at least 10% of patients harboring 9p21.3 deletion were identified. Tumors were classified as 9pS if they had a focal deep deletion of CDKN2A/B. Tumors were classified as 9 pL if both CDKN2A/B and the type I IFN cluster was deleted. For the 9 pL/9pS relative frequency, only datasets with at least 40 cases with 9p21.3 loss were considered.

Cell Culture. NIH3T3 fibroblasts were obtained from the American Type Culture Collection (ATCC), and were cultured in DMEM supplemented with 10% fetal calf serum (FCS) and 100 IU/mL of penicillin/streptomycin. Parental and stably-expressing Gag/Pol HEK293 lines were cultured in DMEM supplemented with 10% fetal bovine serum (FBS) and 100 IU/mL of penicillin/streptomycin. Pancreatic ductal epithelial cells (PDECs), derived from female C57BL/6n mice, were cultured as previously described^{37,38}. Advanced DMEM/F12 supplemented with 10% FBS (Gibco), 100 IU/mL of penicillin/streptomycin (Gibco), 100 mM Glutamax (Gibco), ITS Supplement (Sigma), 0.1 mg/mL soy trypsin-inhibitor (Gibco), Bovine Pituitary Extract (Gibco), 5 nM T3 (Sigma), 100 mg/mL Cholera toxin (Sigma), 4 mg/mL Dexamethasone (Sigma), 10 ng/mL human EGF (Preprotech). PDECs were cultured on collagen-coated plates (100 mg/mL PureCol 5005, Advanced Biomatrix). Tumor-derived cell lines were generated by an initial mechanical disaggregation/mincing, and tumor fragments were transferred to a solution of type V collagenase (Sigma C9263, 1 mg/mL in HBSS 1X) and incubated at 37 C for 45 minutes. Cell suspensions were supplemented with an equal volume of DMEM 10% FBS and filtered through a 100 mm mesh (BD). Filtered suspensions were centrifuged for 5 min at 1250 rpm, pellets were resuspended in DMEM 10% FBS with penicillin/streptomycin 100 ml/mL and cultured on collagen coated plates (100 mg/mL PureCol 5005, Advanced Biomatrix). Cells were passaged twice to remove non-tumor cells and downstream applications were done with these tumor-derived cell lines.

Engineering Large Genomic Deletions with MACHETE. To engineer genomic deletions, we developed Molecular Alteration of Chromosomes with Engineered Tandem Elements (MACHETE). The premise behind MACHETE is to give cells that bear the deletion of interest a selective advantage over unedited cells, which is achieved by using a bicistronic cassette consisting of an inducible suicide element and an antibiotic resistance component. This cassette is integrated into the region of interest by CRISPR-Cas9 mediated homology directed repair (HDR). Once cells with stable integration of the cassette are positively selected, they are treated with CRISPR-Cas9 to generate the deletion of interest and edited cells are enriched via negative selection.

Identification and in vitro transcription of sgRNAs. We used GuideScan to select optimal sgRNA sequences⁶⁵. For each locus of interest, we identified an sgRNA to introduce the MACHETE cassette by HDR, and sgRNAs to generate the deletion of interest. For the 4C4 locus, we designed two independent sets of guides for each deletion to control for potential off-target effects. We generated sgRNAs as previously described ⁶⁶. Briefly, a primer with a T7 adapter and the sgRNA sequence was used to PCR amplify the tracrRNA from a pX330 plasmid. The PCR product was then purified and transcribed using the RNA MAXX In Vitro Transcription Kit (Agilent) to produce the sgRNA. sgRNAs were then column purified (RNA Clean & Concentrator, Zymo Research), eluted in water and aliquoted for later use with recombinant Cas9 (Sigma). Oligos used for sgRNA production are listed in Table A represented as SEQ ID NOs: 1-44 in order of appearance.

TABLE A
Primers

| Primers for sgRNA | |
|---|---|
| synthesis | Sequence (T7 adapter/Protospacer/pX binding site) |
| 11B3 MACHETE KI guide | |
| 11B3 KI | TAATACGACTCACTATAGG TGATGGGGTCCGGTCCTCAA GTTTTAGAGCTAGAAATAGC |
| 11B3 deletion | |
| Sco1 g1 | TAATACGACTCACTATAGG ACCGGCCGGGTGGAACTGCG GTTTTAGAGCTAGAAATAGC |
| Alox12 g1 | TAATACGACTCACTATAGG TTGGCCTCCCTCAAGATGCG GTTTTAGAGCTAGAAATAGC |
| 4C4 MACHETE KI guide | |
| 4C4 KI | TAATACGACTCACTATAGG CCGTAGGCTACCAAACCAGA GTTTTAGAGCTAGAAATAGC |
| 4C4-S 5′ | |
| 4C4 5′ guide 1 | TAATACGACTCACTATAGG CTCGAATTCATTTCTGTTCG GTTTTAGAGCTAGAAATAGC |
| 4C4 5′ guide 2 | TAATACGACTCACTATAGG CTGTAATATCACTCTATAAC GTTTTAGAGCTAGAAATAGC |
| 4C4-L 5′ | |
| Hacd4 guide 1 | TAATACGACTCACTATAGG TCGCGGGAGTGAAAGCCGAT GTTTTAGAGCTAGAAATAGC |
| Hacd4 guide 2 | TAATACGACTCACTATAGG CGTCCTATAATTGAAGCCCG GTTTTAGAGCTAGAAATAGC |
| Dmrta1 (common 3′ guides) | |
| Dmrta1 guide 1 | TAATACGACTCACTATAGG GTGGTGAAACCCGAACTCGT GTTTTAGAGCTAGAAATAGC |
| Dmita1 guide 2 | TAATACGACTCACTATAGG ACATGGTTAGGCTTTACCCC GTTTTAGAGCTAGAAATAGC |
| 404-I 3′ | |
| 4C4-I guide 1 | TAATACGACTCACTATAGG GCGTACTCTTGCCACTTCCG GTTTTAGAGCTAGAAATAGC |
| 4C4-I guide 2 | TAATACGACTCACTATAGG ATAGCCAGATTTGCAGACGC GTTTTAGAGCTAGAAATAGC |
| 4C4 allelic series | |
| 4C4-del B guide | TAATACGACTCACTATAGG GATGGCGCTTGAATACTGGA GTTTTAGAGCTAGAAATAGC |
| 4C4-del C guide | TAATACGACTCACTATAGG ACATAGGTTGGTGGCGGAGT GTTTTAGAGCTAGAAATAGC |
| 4C4-del D guide | TAATACGACTCACTATAGG AACCTCCCCACGTAATAGAA GTTTTAGAGCTAGAAATAGC |
| 4C4-del E guide | TAATACGACTCACTATAGG CCGCCATGTCCGCCAAGAGA GTTTTAGAGCTAGAAATAGC |
| Universal Reverse primer | AGCACCGACTCGGTGCCACT |
| Primers for HDR donors | Homology Arm/MACHETE plasmid binding |
| 11B3 Donor | |
| 5′ HA PGK F | AACAGAAGTCGGTGAATAGAATTTAAATTTCCCTTGAGGA AACGAATTCTACCGGGTAGG |
| 3′ HA bGHpA R | ATCAAAGAGCTTGCCTGATGCTCCCTGTGATGGGGTCCGG CCATAGAGCCCACCGCATCC |
| 4C4 Donor | |
| 5′ HA PGK F | TATAACAGGGGACACCACCTCTTCCAGAGACCGTAGGCTA AACGAATTCTACCGGGTAGG |
| 3′ HA bGHpA R | GAAGATGCTGCTTATACTTGGCATTAGCCTTCTGGTTTGG CCATAGAGCCCACCGCATCC |
| 7q11 Donor | |
| 5′ HA Dm g1 F | GTTCTGCAGTCATGTGGAAGATGGAGGCCCTGCACGCTAC CAGGAGCTATTAATTCGCGG |
| 3′ HA Dm g2 R | GTTTTCGCAGCACTGGTGTCACCTCGTCCTGCCCAACTAT CCATCTTCGGGAACATCCGG |

Primers for Genotyping

| | |
|---|---|
| 118B KI | |
| 11B3 KI F | CAAGGAAAGTCAAGCCCAGA |
| PGK R | CTGTCCATCTGCACGAGACT |
| 11B3 deletion | |
| Δ11B3 F | GGGCAAAGGTGAGAAAACAA |
| Δ11B3 R | ATCCGAGATCTGGAGGAGGT |
| 4C4 KI | |
| 4C4 KI F | GGGATACCCCTCAACAGGAT |
| PGK R | CTGTCCATCTGCACGAGACT |
| 4C4-S Deletion | |
| 4C4 KI F | GGGATACCCCTCAACAGGAT |
| 4C4-S Deletion 1 R | CCTCATAACAGTGGCCTGCT |
| 4C4-S Deletion 2 R | GCTGTCCAGGGAGTTAGAGG |
| 4C4-L Deletion | |
| 4C4-L Deletion 1 F | TACCAGGCAGAAGGAGCAGT |
| 4C4-L Deletion 1 R | ACCTTGCGGTATGGAGTTTG |
| 4C4-L Deletion 2 F | ACTCTCAGGGTTCCCCAAGT |
| 4C4-L Deletion 2 R | AAGCCACAGTAGGGCTCAGA |
| 4C4-I Deletion | |
| 4C4-I Deletion 1 F | TACCAGGCAGAAGGAGCAGT |
| 4C4-I Deletion 1 R | ACCTTGCGGTATGGAGTTTG |
| 4C4-I Deletion 2 F | ACTCTCAGGGTTCCCCAAGT |
| 4C4-I Deletion 2 R | AAGCCACAGTAGGGCTCAGA |
| TK (internal primers) | |
| TK F | TCACATGCCCCGCCCCCGGC |
| TK R | TTAGTTAGCCTCCCCCATCTCCC |
| 7q11-22 Deletion | |
| 7q11 Del F | GGCATGCCTGCTATTTTGAT |
| 7q22 Del R | GGGAGCAACCTCGGTGATA |

Generation of HDR donor: To maximize flexibility, MACHETE uses 40-bp homology arms that are introduced by PCR. The locus-specific HIDR donors were generated by PCR amplification of the MACHETE bicistronic cassette using a high-fidelity DNA polymerase (Herculase II, Agilent or Q5, NEB). PCR fragments were column purified (Qiagen) and quantified. Primers for targeting are presented in Table A.

CRISPR-Cas9-mediated targeting and generation of large genomic deletions: For all CRISPR editing, we used Cas9 ribonucleotide complexes (Cas9 RNPs) with the intended guides, to reduce cloning and limit Cas9 expression. To incorporate Cas9 RNPs and donor PCR, cells were electroporated with the Neon System (Invitrogen) following the manufacturer's instructions.

HDR knock-in of MACHETE cassette: Briefly, cells were trypsinized, washed in PBS once, and counted. Cells were then resuspended in Neon Buffer R and aliquoted for the different electroporation reactions. Each condition used 100×10³ cells in 10 mL of Buffer R. In parallel, 1 mg of Cas9 (ThermoFisher) and 1 mg of sgRNA were complexed for 15 min at room temperature. For the HDR step, 0.5 mg of donor DNA was added to the Cas9 RNP complex, which was then mixed with the cell aliquot. The cell/RNP/donor mixture was electroporated (1400 V pulse voltage, 20 ms pulse width, 2 pulses). For the selection of cassette knock-in lines, Puromycin (2 mg/mL) was added to the media 48 hours after electroporation. In the case of fluorescence reporters, cells were sorted 48 hours post electroporation (Sony MA900), and further enriched for stable expression one week after this initial sort. Selected cells were expanded to establish the parental KI lines. To validate this initial step, cells were then treated with diphtheria toxin (50 ng/mL) or ganciclovir (10 mg/mL) to assess their sensitivity. On-target integrations were assessed by PCR of gDNA and Sanger sequencing of the product for confirmation. Genotyping primers are provided in Table A.

Generation of genomic deletions: KI cells were trypsinized, washed in PBS once, and counted. Cells were then resuspended in Neon Buffer R and aliquoted for the different electroporation reactions. Each condition used 100×10³ cells in 10 mL of Buffer R. In parallel, 2 mg of Cas9, 1 mg of 5′ flanking sgRNA, and 1 mg of 3′ flanking sgRNA were complexed for 15 min at room temperature. The cell/RNP mixture was electroporated (1400 V pulse voltage, 20 ms pulse width, 2 pulses) and cells were seeded in the absence of selection. 48 hours after seeding, cells were treated with diphtheria toxin (50 ng/mL) or ganciclovir (10 mg/mL) and media was changed every 2 days with ongoing selection. Surviving cells were then passaged and analyzed for the presence of the intended deletion breakpoint, loss of selection cassette, and sensitivity to selection was re-evaluated. Genotyping primers are provided in Table A.

Breakpoint high-throughput sequencing: Breakpoint PCRs were purified and sent for amplicon sequencing (Amplicon-EZ, Genewiz) following service guidelines. Raw fastq reads were aligned to the mouse genome (mm 10) using bowtie2 with parameters “—local-D 50-R 3-NO-L 19-i S, 1.0,0.7—no-unal-k 5—score-min C,20”. Aligned SAM reads were processed using custom Rscript to parse the breakpoint location, junction position, direction of the reads, and alignment types. Alignments for a proper break read-pairs have to both aligned to the same breakpoint chromosome; coming from 1 primary and 1 secondary alignment; and breakpoints must be located on opposite sides of the breakpoint junction.

Flow Cytometry. To assess expression of EGFP, tumor cell suspensions were generated by initial mechanical disaggregation/mincing. Tumor fragments were then transferred to a solution of type V collagenase (Sigma C9263, 1 mg/mL in 1X HBSS) supplemented with soy trypsin inhibitor (Gibco, 0.1 mg/mL) and DNAse I (Sigma, 0.1 mg/mL). Tumor pieces in this disaggregation buffer were transferred to a GentleMACS tube and loaded into the OctoDissociator (Miltenyi). Samples were treated with the mTDK1 program, after which 5 mL of FACS Buffer (PBS 1X, 2% FBS) was added to the sample and the mix was filtered through a 100 mm mesh (BD). The resulting cell suspension was centrifuged and resuspended in FACS buffer. Cells were then treated with Fc block (BD, 1:200 dilution) incubated at 4C for 15 minutes and stained with anti-CD45 AF700 (BD, 1:400 dilution) for 30 min at 4C. Cells were washed and resuspended in FACS buffer supplemented with DAPI (Sigma, 1 mg/mL final). Stained cell suspensions were then analyzed in a MA900 sorter (Sony). EGFP+ cells were analyzed within the CD45-, DAPI-population.

For multi-parametric flow cytometry analysis, tumor cell suspensions were generated as above, and cells were stained with LIVE/DEAD fixable viability dye (Invitrogen) for 30 min at 4C. After this, cells were washed, incubated with Fc block (BD, 1:200) for 15 min at 4 C, and then stained with conjugated antibody cocktails (see Table B for antibody panels) for 30 min at 4C.

TABLE B
Flow Cytometry Panels

| | | | | |
|---|---|---|---|---|
| Marker | Fluorophore | Clone | Company | Concentration |

Lymphoid

| | | | | |
|---|---|---|---|---|
| CD45 | AF700 | 30-F11 | BioLegend | 1/400 |
| CX3CR1 | BV510 | SA011F11 | BioLegend | 1/400 |
| CD3 | PE Fluo610 | 145-2C11 | eBioscience | 1/100 |
| CD4 | BV605 | RM4 5 | BD | 1/200 |
| CD8 | PE Cy7 | 53-6.7 | BioLegend | 1/400 |
| PD-L1 | APC Cy7 | 10F.9G2 | BioLegend | 1/400 |
| CD44 | BV786 | IM7 | BioLegend | 1/400 |
| CD69 | BUV737 | H1.2F3 | BD | 1/400 |
| CD19 | BV650 | 1D3 | BD | 1/400 |
| PD1 | PE | 29F.1A12 | BioLegend | 1/400 |
| CD62L | APC Cy7 | MEL-14 | BioLegend | 1/400 |
| TCR gd | BUV395 | V65 | BD | 1/200 |
| Viability | BV421 | Live/Dead | Invitrogen | 1/1000 |
| Fc Block | NA | 2.4G2 | BD | 1/200 |

Myeloid

| | | | | |
|---|---|---|---|---|
| CD45 | AF700 | 30-F11 | BioLegend | 1/400 |
| CD11b | BUV395 | M1/70 | BD | 1/800 |
| CD86 | BV650 | GL-1 | BioLegend | 1/400 |
| Ly6C | APC Cy7 | AL-21 | BD | 1/400 |
| Ly6G | BV605 | 1A8 | BD | 1/400 |
| CD11c | BV786 | N418 | BioLegend | 1/800 |
| CD206 | PerCP Cy5.5 | C068C2 | BioLegend | 1/400 |
| F4/80 | APC | BM8 | BioLegend | 1/400 |
| CD103 | PE-Cy7 | 2E7 | BioLegend | 1/400 |
| CX3CR1 | BV510 | SA011F11 | BioLegend | 1/400 |
| CD8 | PE Cy7 | 53-6.7 | BioLegend | 1/400 |
| Viability | BV421 | Live/Dead | Invitrogen | 1/1000 |
| Fc Block | NA | 2.4G2 | BD | 1/200 |

After staining cells were washed and fixed (BD Cytofix) for 20 min at 4C, washed again, and stored for analysis. Samples were analyzed in a BD LSRFortessa with 5 lasers, where gates were set by use of fluorescence-minus-one (FMO) controls.

### Animals and In Vivo Procedures

Animals. All mouse experiments were approved by the Memorial Sloan-Kettering Cancer Center (MSKCC) Institutional Animal Care and Use Committee (IACUC). Mice were maintained under pathogen-free conditions, and food and water were provided ad libitum. C57Bl/6n and NOD/SCID I12rg−/− (NSG) mice were purchased from Envigo. Foxn 1^{nu} (Swiss nude) mice were purchased from Jackson Laboratory. All mice used were 6 to 8 week-old females.

PDAC GEMM-ESC models of Cdkn2a b loss. Embryonic stem cells (ESCs) bearing alleles to study PDAC were used as previously described⁶⁷⁻⁶⁹. Briefly, Ptf1a^{Cre/+}; Rosa26^{Lox-Stop-Lox rt′TA3-IRES-mKate2/+}; Col1a1^{Homing cassette/+} cells were targeted with shRNAs against Smad4 or Renilla luciferase (non-targeting control). Mice were then generated by blastocyst injection of shSmad4 or shRen ESCs, and shRNAs were induced by treatment of the resulting mice with doxycycline in drinking water starting at 5-6 weeks of age. Pancreatic tumor initiation and progression were monitored by palpation and ultrasound imaging, mice were euthanized upon reaching humane endpoints of tumor burden, and samples were collected from primary tumors and metastases (when present). Tumor-derived cell lines were then analyzed by sparse whole genome sequencing and classified according to the type of Cdkn2a b alteration.

Orthotopic transplants. For orthotopic transplants of PDEC cells, mice were anesthetized and a survival surgery was performed to expose the pancreas, where either 300,000 cells (for primary MACHETE-edited lines) or 100,000 cells (tumor-derived lines) were injected in the pancreas of each mouse. Mice were then monitored for tumor engraftment (bioluminescence imaging, IVIS) and progression, and were euthanized when overt disease was present in accordance with IACUC guidelines.

Experimental metastasis assays. For liver colonization of PDEC cells, mice were anesthetized, and a survival surgery was performed to expose the spleen, where 100,000 cells (tumor-derived lines) were injected in the spleen of each mouse, where the site of injection was then removed and the remainder of the spleen was cauterized (hemi-splenectomy). Mice were then monitored for tumor engraftment and progression and were euthanized when overt disease was present in accordance with IACUC guidelines.

Antibody treatments. For IFNARI blockade experiments, mice were treated twice per week with either 200 ug i.p. of control IgG (MOPC21 clone, BioXCell) or 200 ug i.p. of anti-IFNARI antibody (MAR15A3, BioXCell). For depletion experiments: mice were treated with anti-CD8a antibody (Clone 2.43, BioXCell) or anti-CD4 (Clone GK1.5, BioXCell) with an initial dose of 400 ug i.p., followed by maintenance injections of 200 ug/mouse. Control, IFNAR1 blocking and CD8/CD4 depletion antibody treatments were done twice per week, starting one week prior to the orthotopic transplantation of cells. Treatments were maintained for the entire duration of the experiment. B cell depletion was done by a monthly intravenous injection of anti-CD20 (Clone SA271G2, BioLegend), starting one week prior to orthotopic transplantation of cells.

In vivo bioluminescence imaging. Mice were anesthetized and hair over the imaging site was removed. Mice were injected with 200 μL of luciferin i.p. (200 mg/L, PerkinElmer #122799) and bioluminescence was acquired 10 minutes after the luciferin injection in an IVIS Spectrum. For organ imaging, mice were injected with luciferin, euthanized 10 min after the injection, and organ bioluminescence was acquired in an IVIS Spectrum instrument.

Imaging and assessment of metastatic burden. Mice meeting endpoint criteria were euthanized and inspected for overt macro-metastatic burden in the abdominal cavity (peritoneum, diaphragm, mesenteric lymph nodes, ovary/fallopian tubes, kidneys, and liver), as well as in the thoracic cavity (lungs and rib cage). Primary tumors and organs were dissected and imaged under a dissection microscope (Nikon SMZ1500) for brightfield and EGFP fluorescence.

RNA Extraction and cDNA Preparation. RNA was extracted by using the Trizol Reagent (ThermoFisher) following the manufacturer's instructions. The only modification was the addition of glycogen (40 ng/mL, Roche) to the aqueous phase to visualize the RNA pellet after precipitation. RNA was quantified using a Nanodrop. cDNA was prepared with the AffinityScript QPCR cDNA Synthesis Kit (Agilent) following the manufacturer's instructions.

DNA Extraction. Genomic DNA was extracted from cells or tissues using the DNeasy Blood and Tissue Kit (Qiagen) following the manufacturer's instructions.

qPCR. For quantitative PCR the PerfeCTa SYBR Green FastMix (QuantaBio), the Taqman Fast Advanced Master Mix (Applied Biosystems), and the Taqman Genotyping Master Mix (Applied Biosystems) were used following manufacturer's instructions. For qPCR primers and Taqman assays, see Table C.

TABLE C
Primers and Taqman assays for qPCR
RT qPCR primers

| | | |
|---|---|---|
| Actb F | GGCTGTATTCCCCTCCATCG | (SEQ ID NO: 45) |
| Actb R | CCAGTTGGTAACAATGCCATGT | (SEQ ID NO: 46) |
| Gapdh F | GGGAAATTCAACGGCACAGT | (SEQ ID NO: 47) |
| Gapdh R | AGATGGTGATGGGCTTCCC | (SEQ ID NO: 48) |
| EGFP F | ACGTAAACGGCCACAAGTTC | (SEQ ID NO: 49) |
| EGFP R | AAGTCGTGCTGCTTCATGTG | (SEQ ID NO: 50) |
| Ifnb1 F | CAGCTCCAAGAAAGGACGAAC | (SEQ ID NO: 51) |
| Ifnb1 R | GGCAGTGTAACTCTTCTGCAT | (SEQ ID NO: 52) |
| Ifne F | GACAGCTCCCTGAAACGGTG | (SEQ ID NO: 53) |
| Ifne R | GGCTTTCTCTGTTCATCCACA | (SEQ ID NO: 54) |
| Isg20 F | TGGGCCTCAAAGGGTGAGT | (SEQ ID NO: 55) |
| Isg20 R | CGGGTCGGATGTACTTGTCATA | (SEQ ID NO: 56) |
| Oas11 F | CAGGAGCTGTACGGCTTCC | (SEQ ID NO: 57) |
| Oasl1 R | CCTACCTTGAGTACCTTGAGCAC | (SEQ ID NO: 58) |

Taqman Assays

| | |
|---|---|
| Actb | Mm02619580_g1 |
| Gapdh | Mm99999915_g1 |
| Ifnb1 | Mm00439552_s1 |
| Ifna13 | Mm01731013_s1 |
| Ifna5 | Mm00833976_s1 |
| Ifna4 | Mm00833969_s1 |
| Ifna7 | Mm02525960_s1 |
| Ifne | Mm00616542_s1 |

Copy Number Taqman Assays

| | |
|---|---|
| Tfrc | 4458366 |
| Cdkn2a | Mm00556883_cn |
| Mtap | Mm00127621_cn |
| Ifne | Mm00555452_cn |
| Klh19 | Mm00558886_cn |
| Ifna13 | Mm00658525_cn |
| Ifnb1 | Mm00555445_cn |

Histology. Tissues were formalin fixed, dehydrated and paraffin embedded for sectioning. Hematoxylin/Eosin staining was performed with standard protocols.

RNA Sequencing, Differential Gene Expression, and Gene Set Enrichment Analysis. Bulk tumor pieces were flash frozen on dry ice and stored at −80C. Tissues were then mechanically disrupted in Trizol and RNA was extracted following manufacturer's instructions. RNA integrity was analyzed with an Agilent 2100 Bioanalyzer. Samples that passed QC were then used for library preparation and sequencing. Samples were barcoded and run on a HiSeq (Ilumina) in 76 bp SE run, with an average of 50 million reads per sample. RNA-Seq data was then trimmed by removing adapter sequences and reads were aligned to the mouse genome (GRCm38.91; mm 10), and transcript counts were used to generate an expression matrix. Differential gene expression was analyzed by DESeq2⁷⁰ for 3-5 independent tumors per condition. Principal Components Analysis (PCA) and differentially expressed gene analysis was performed in R, with significance determined by >2 fold change with an adjusted p value <0.05. GSEA^{71,72} was performed using the GSEAPreranked tool for conducting GSEA of data derived from RNA-seq experiments (v.2.07) against specific signatures: Hallmark Pathways, Reactome Pathways, and Immune Subpopulations.

Sparse Whole Genome Sequencing. Low-pass whole genome sequencing was performed on gDNA freshly isolated from cultured cells as previously described ⁷³. Briefly, 1 mg of gDNA was sonicated on an E220 sonicator (settings: 17Q, 75s Covaris), and library preparation was done by standard procedure (end repair, addition of polyA, and adapter ligation). Libraries were then purified (AMPure XP magnetic beads, Beckman Coulter), PCR enriched, and sequenced (Illumina HiSeq). Reads were mapped to the mouse genome, duplicates removed, and an average of 2.5 million reads were used for CNA determination with the Varbin algorithm⁷⁴.

Human PDAC Transcriptional Analysis. Samples from the COMPASS trial^{44,45} were classified as primary or metastatic disease and further subdivided by status of the 9p21.3 locus: 9pS deletion affecting CDKN2A/B, or 9 pL deletions affecting CDKN2A/B and at least one IFN gene from the linked cluster. 9pS and 9 pL samples were then analyzed for differentially expressed genes using DESeq2 and assessed by GSEA for Reactome Pathways⁷⁵, and Immune Subpopulations⁴². As an independent validation of the differences between primary and metastatic PDAC, a previously published dataset⁷⁶ was used to derive differentially expressed genes using DESeq2. Genes downregulated in PDAC metastasis were then analyzed using the Enrichr algorithm⁷⁷.

scRNA Sequencing. The single-cell RNA-Seq of FACS-sorted cell suspensions was performed on Chromium instrument (10X genomics) following the user guide manual for 3′ v3.1. In brief, FACS-sorted cells were washed once with PBS containing 1% bovine serum albumin (BSA) and resuspended in PBS containing 1% BSA to a final concentration of 700-1,300 cells per μl. The viability of cells was above 80%, as confirmed with 0.2% (w/v) Trypan Blue staining (Countess II). Cells were captured in droplets. Following reverse transcription and cell barcoding in droplets, emulsions were broken and cDNA purified using Dynabeads MyOne SILANE followed by PCR amplification per manual instruction. Between 15,000 to 25,000 cells were targeted for each sample. Samples were multiplexed together on one lane of 10X Chromium following cell hashing protocol⁷⁸. Final libraries were sequenced on Illumina NovaSeq S4 platform (R1-28 cycles, i7-8 cycles, R2-90 cycles). The cell-gene count matrix was constructed using the Sequence Quality Control (SEQC) package⁷⁹.

Data Pre-processing. FASTQ files were generated from 3 different samples (ΔL, ΔS, a-IFNARI ΔS) with three mice pooled together per condition. These files were then processed using the SEQC pipeline⁷⁹ using the default parameters for a 10X single-cell 3′ library. This pipeline begins with aligning the reads against the provided mouse mm 10 reference genome and resolving multi-mapping incidents. SEQC then corrects for UMIs and cell barcodes and filters cells with high mitochondrial fraction (>20%), low library complexity (few unique genes expressed), and empty droplets. The resulting count matrix (cell x gene) was generated for each condition as the raw expression matrices.

As each mouse was barcoded with a unique hashtag oligo for each sample, in order to demultiplex the cells, an in-house method known as SHARP (github.com/hisplan/sharp) was employed. Labels are assigned to either identify a cell as belonging to a specific mouse or as a doublet/low-quality droplet. The labeled cell barcodes and gene expression matrix were then concatenated together into one count matrix. Most of the downstream analysis and processing was done using the Scanpy software⁸⁰.

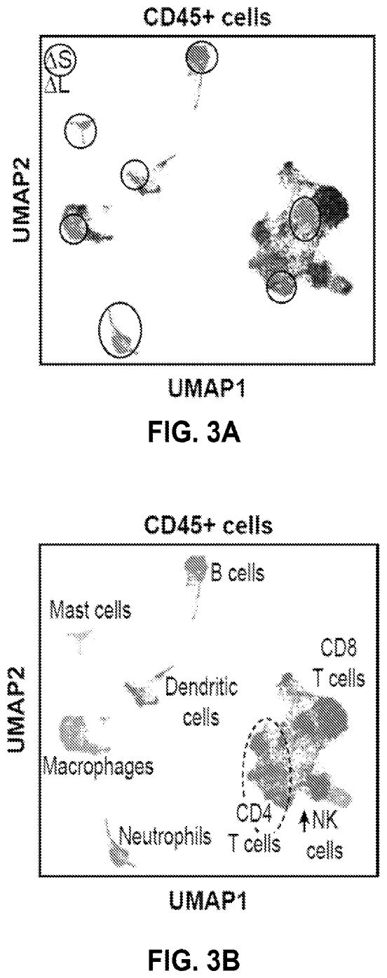

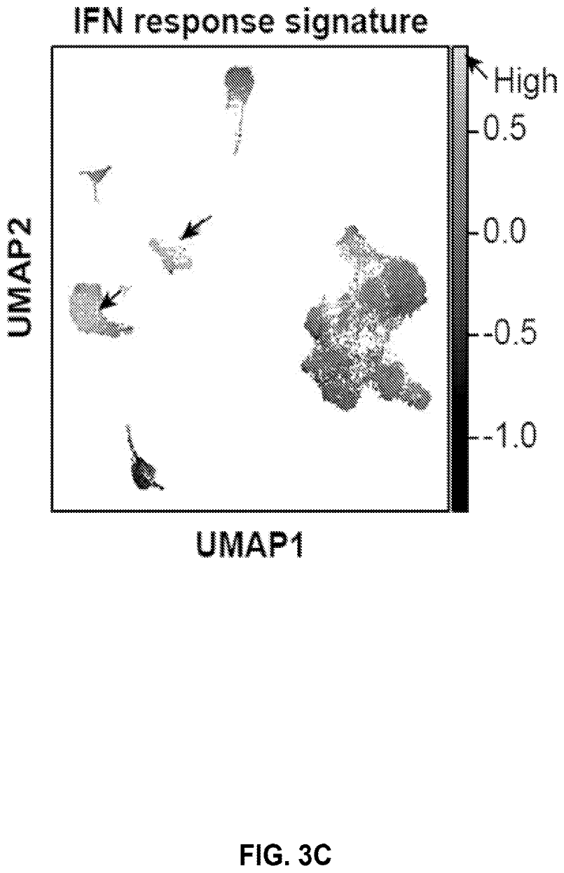

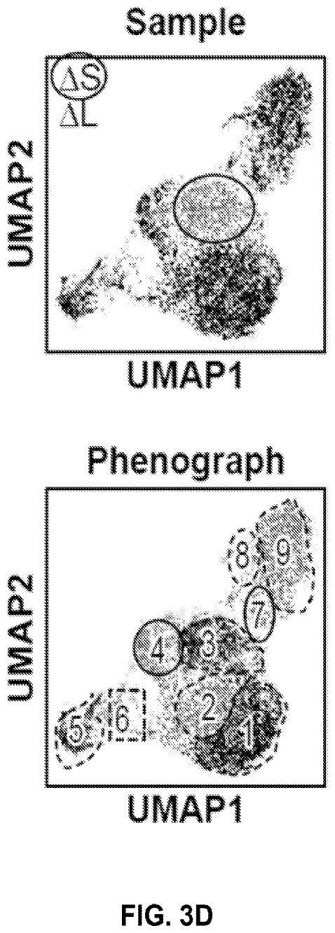

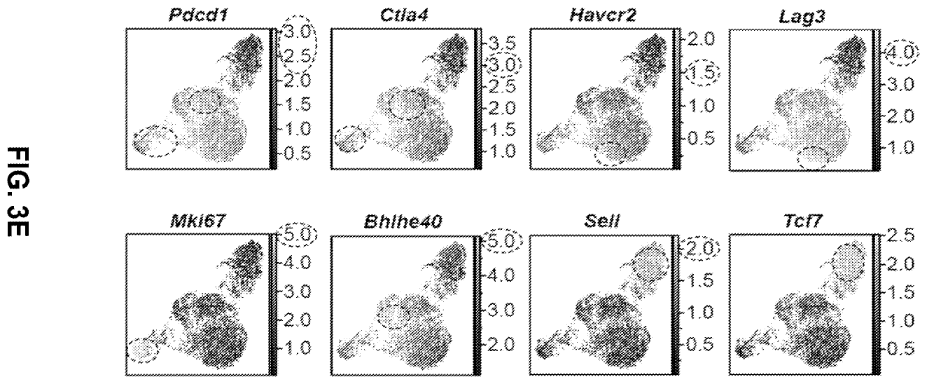

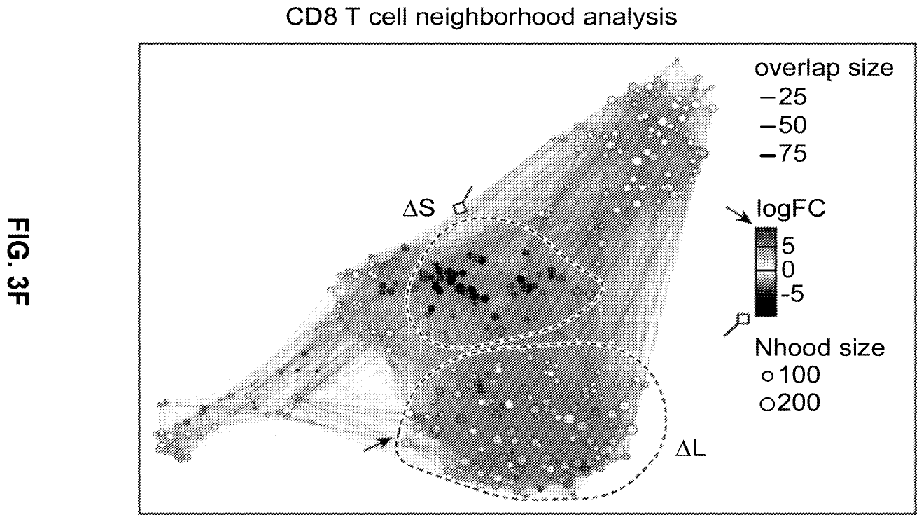

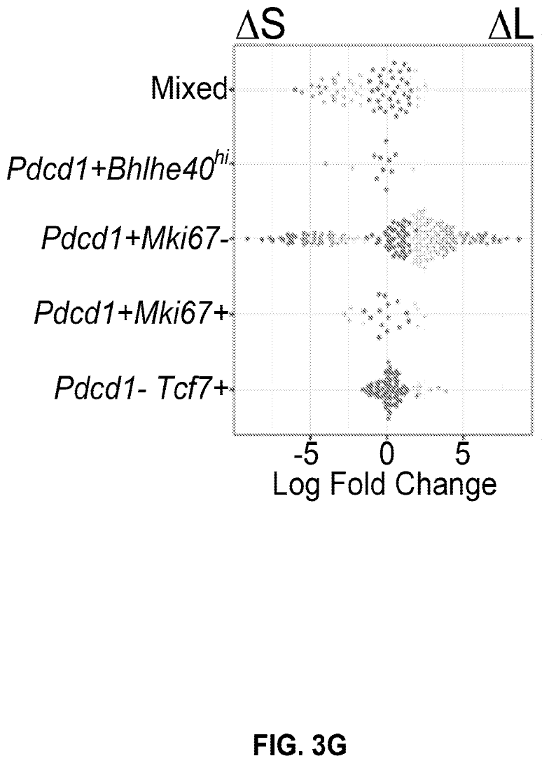

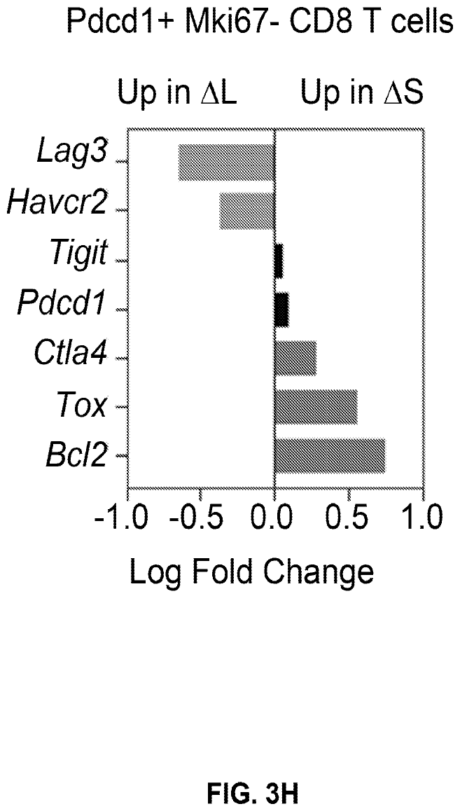

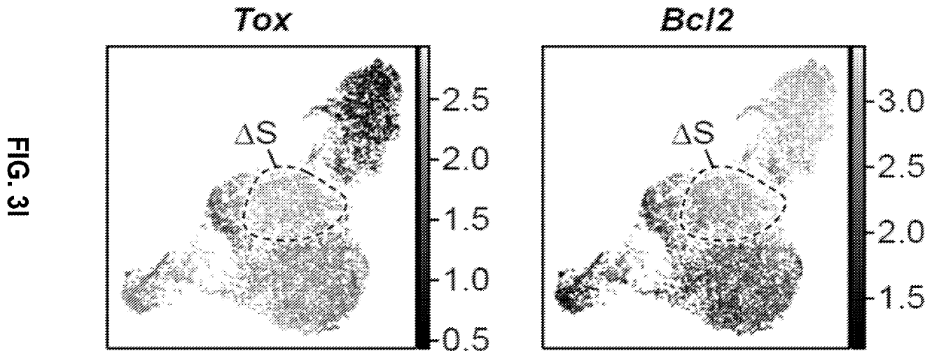

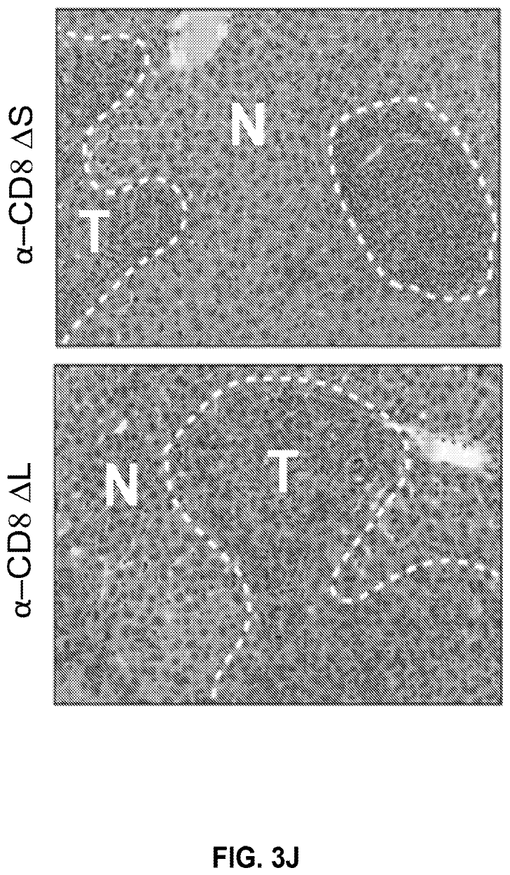

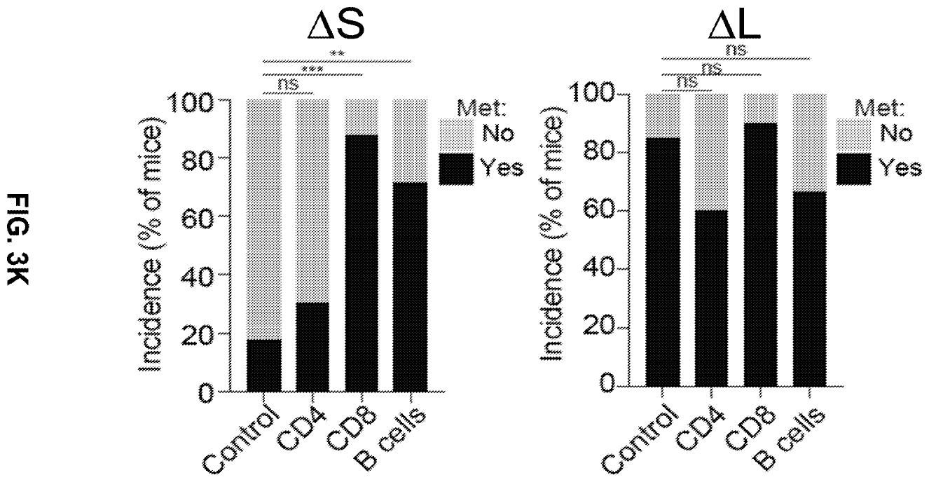

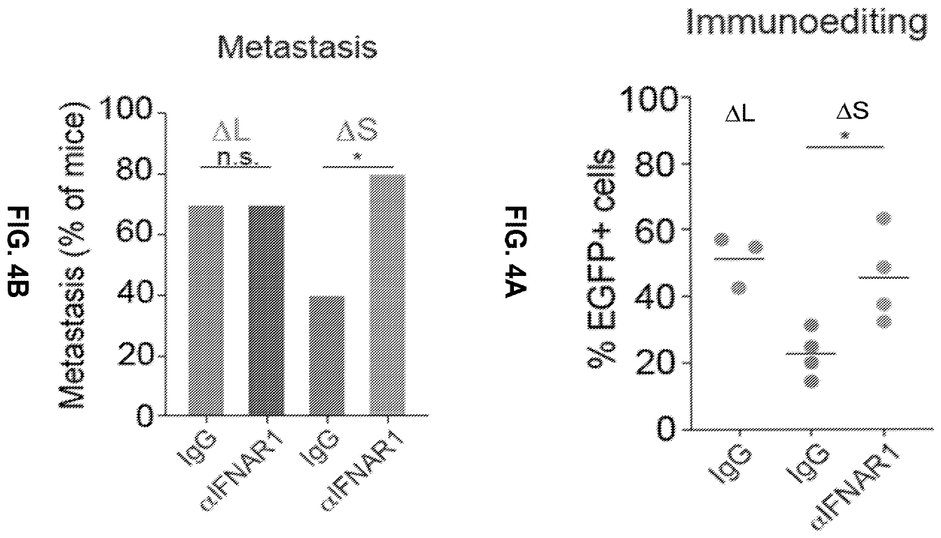

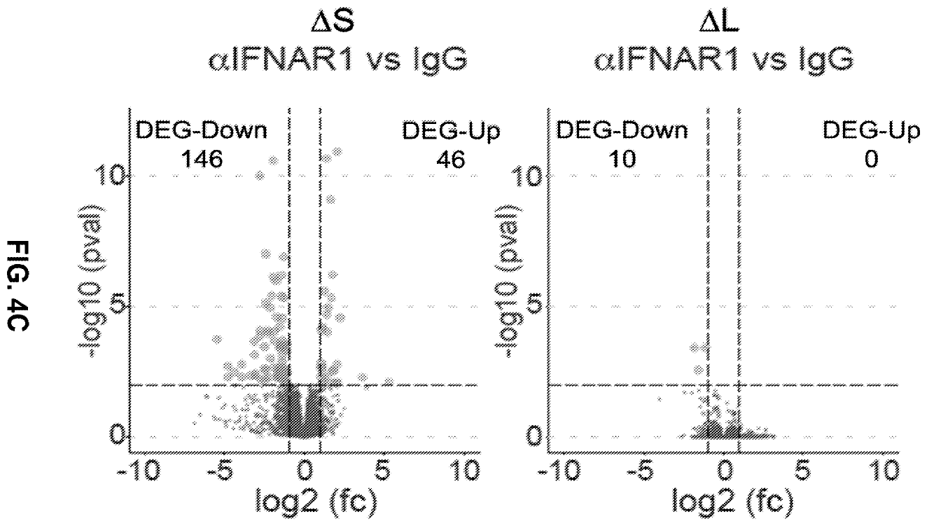

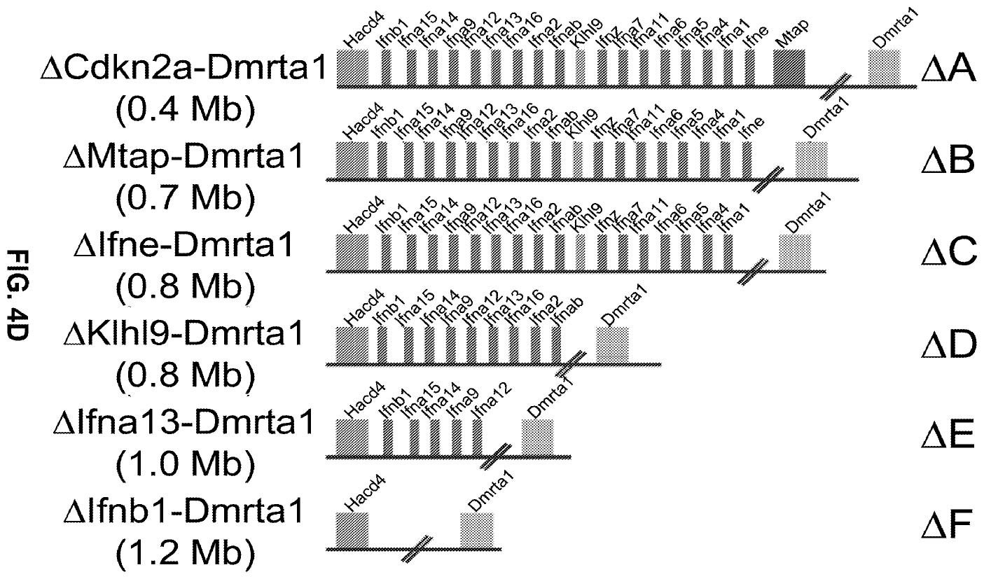

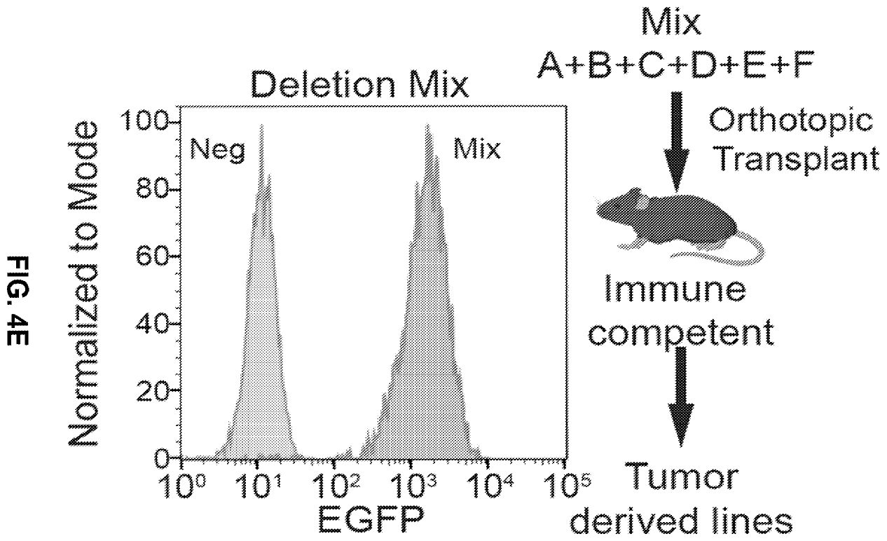

Data cleanup. We began by filtering for lowly expressed genes defined as those expressed in less than 32 cells in the combined dataset. The resulting count matrix was then normalized by library size (defined as the total RNA counts per cell), scaled by median library size, and log 2-transformed with a pseudocount of 0.1 for the combined dataset. For downstream analysis, we first performed dimensionality reduction using Principal Component Analysis (PCA) to obtain top 30 principal components (PCs), chosen based on the decay of associated eigenvalues, computed on the top 4,000 highly variable genes (HVGs). We then computed a k-nearest neighbor graph representation of the cells on the obtained principal components (n_neighbors 30). We visualized the cells on a 2-dimensional projection using UMAP⁸¹ based on the implementation in Scanpy (using min_dist=0.1 parameter). All the cells from different samples were observed to group together based on their cell type, which indicated that no batch effect was present in the data (FIG. 3A). The cells were then clustered using PhenoGraph⁸² on the PCA space with k=30. We ensured that the clusters were robust to variations around the chosen parameter of k. We measured consistency using adjusted rand index (as implemented in the Sklearn package in Python) and observed high degree of consistency for values of k around 30. Upon close inspection of the obtained clusters, we observed one cluster that had low CD45 (PTPRC-) and high KRT8+expression and two other clusters that had low CD45 and high expression of Mitochondrial genes. As such, we decided to remove these clusters from further analysis.

IEN response signature. We first sought to broadly understand, on a per cell type basis, the response to IFN activity. We reasoned that to answer this, we ought to identify the genes that are most differential between a-IFNARI and control ΔS. As such, we constructed an IFN signature by identifying top 100 differentially upregulated genes in ΔS compared to a-IFNAR1. The differential genes were identified using MAST⁸³ and the top 100 genes were averaged on a per cell basis and plotted on the UMAP (FIG. 3C). Once the signature was constructed, we removed cells from the a-IFNARI condition from further analysis in order to directly contrast ΔS and ΔL.

Analysis on ΔS and ΔL samples. The count matrix of CD45+ cells from the AS and ΔL samples included 15334 cells and 15329 genes, 7774 cells belonging to ΔS and 7560 to ΔL. To ensure that the observed heterogeneity was not impacted by these cell clusters, we re-processed the data using the same parameters as described above. Broad cell types were assigned to these clusters according to the average expression of known markers.

CD8. T cells. We isolated cells identified as CD8+ T cells in order to analyze them separately. For this, the 6,080 cells were sub-clustered using PhenoGraph on top of the first 30 PCs (k 30) using 1,500 highly variable genes. Using known markers, these PhenoGraph clusters were then annotated into further subtypes of CD8+ T cells based on the average expression of the markers in each sub-cluster.

Milo analysis on CD8. T cells. We employed Milo⁴³ to statistically quantify the changes in abundance of ΔS and ΔL specific cells among the CD8+ T cells subtypes. Milo utilizes nearest-neighbor graphs to construct local neighborhoods (possibly overlapping) of cells and calculates and visualizes differential abundance of cells from different conditions in the obtained neighborhoods. For this analysis, we first constructed a k-nearest neighbor graph (k=30) on the first 30 PCs using the buildGraph function in Milo. Neighborhoods were calculated using the makeNhoods function (prop=0.1, refined=TRUE). We used default parameters for countCells, testNhoods, and calcNhoodDistance in order to calculate statistical significance and spatial FDR correction, and plotNhoodGraphDA (alpha=0.5) to visualize the results. The color scale of the log FC uses blue to represent higher abundance of ΔL cells and red to represent higher abundance of ΔS specific cells, and the size of the circle is proportional the number of cells belonging to the neighborhood. We further assigned each neighborhood a cell-type identity if more than 80% of the cells in a neighborhood belonged to a specific CD8+ T subtype, otherwise they are categorized as Mixed.

Dendritic cells. Cells annotated as dendritic cells were isolated for further analysis. The 1,134 cells were clustered using PhenoGraph on top 30 principal components (k=30) using 1,500 HVGs. The dendritic cells were further cell typed according to markers from previous studies⁸⁴. The proportion of cells that belong to ΔL and ΔS in each cluster was calculated and plotted.

Macrophages. Cells labeled as macrophages (1,788 cells) were isolated. The cells were clustered using PhenoGraph on top 30 principal components (k=30) using 1,500 HVGs. These clusters were analyzed and annotated according to macrophage subtypes based on the differentially expressed genes computed in each cluster compared to the rest of the data using MAST. The proportion of cells that belong to ΔL and ΔS in each cluster was calculated and plotted.

B cells. 1,204 cells annotated as B cells were selected for. The cells were clustered using PhenoGraph on top 30 principal components (k=30) using 1,500 HVGs. We obtained differentially expressed genes in each B cell sub-cluster using MAST and utilized the results to distinguish distinct populations. The proportion of cells that belong to ΔL and ΔS in each cluster was calculated and plotted.

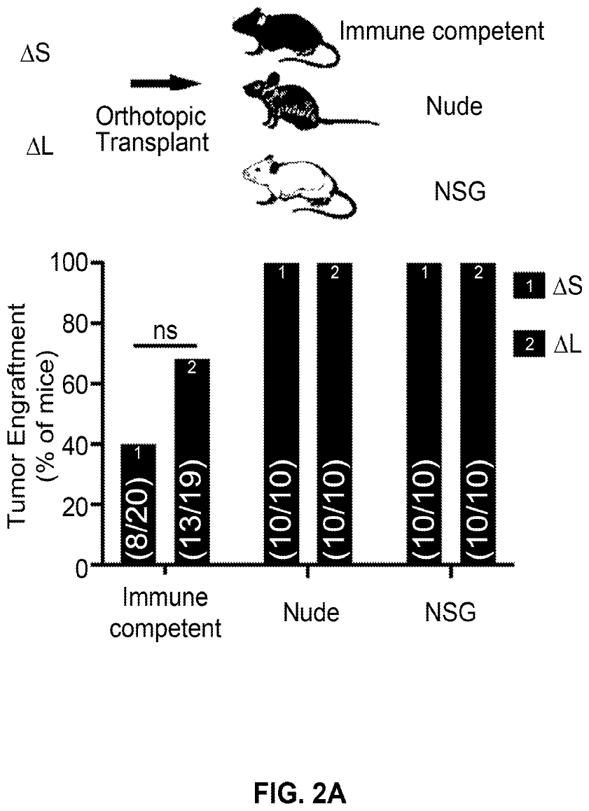

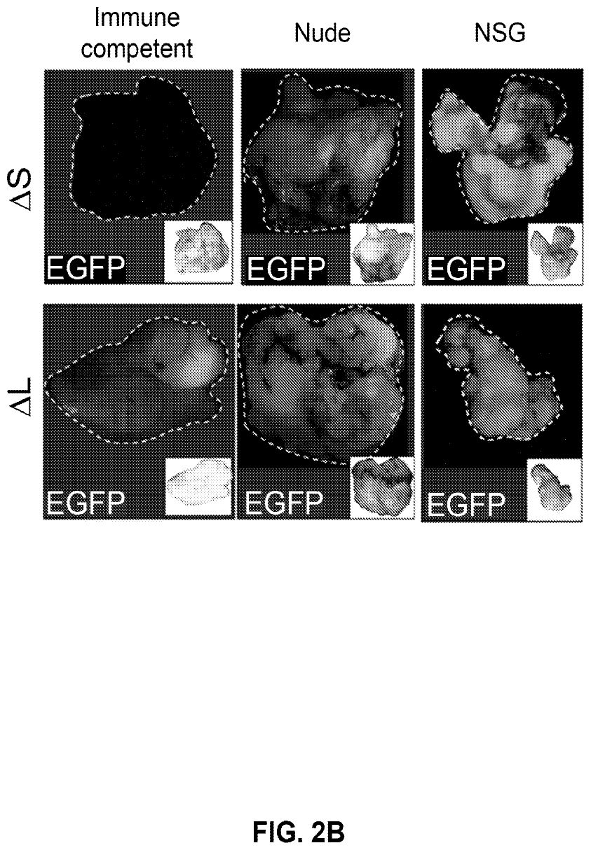

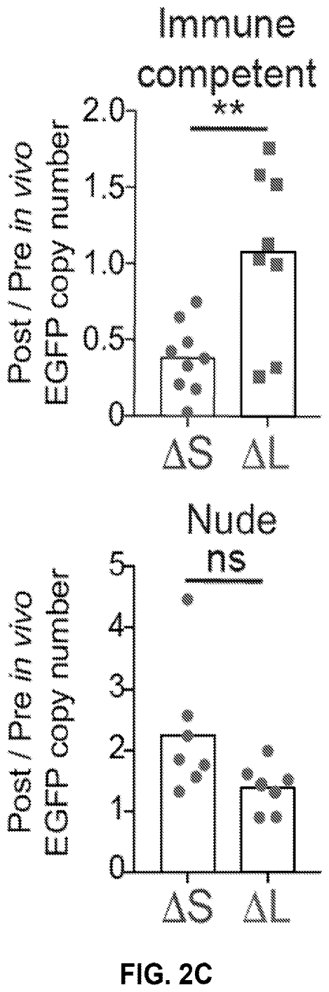

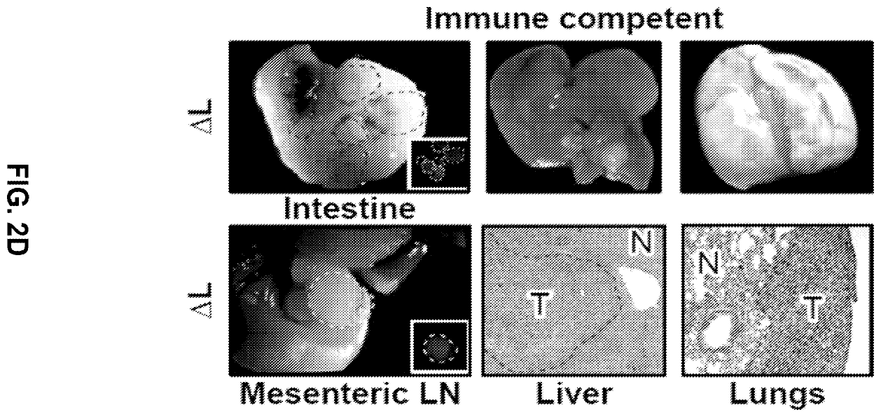

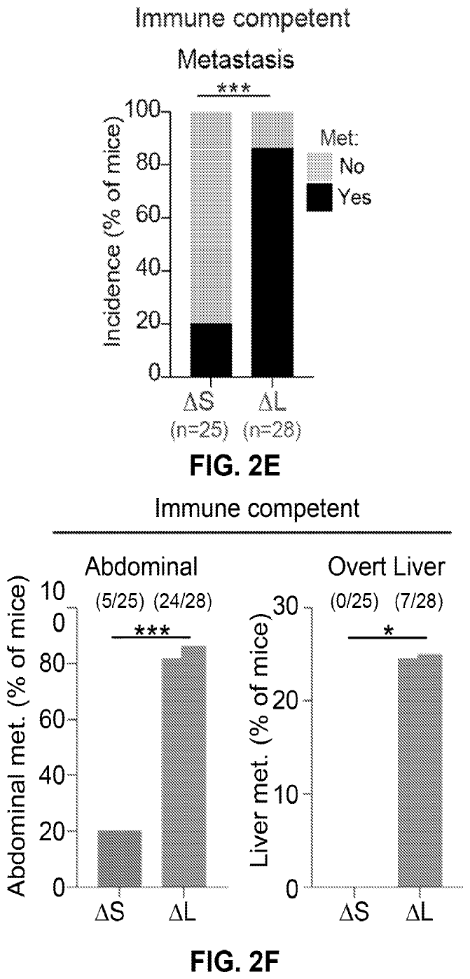

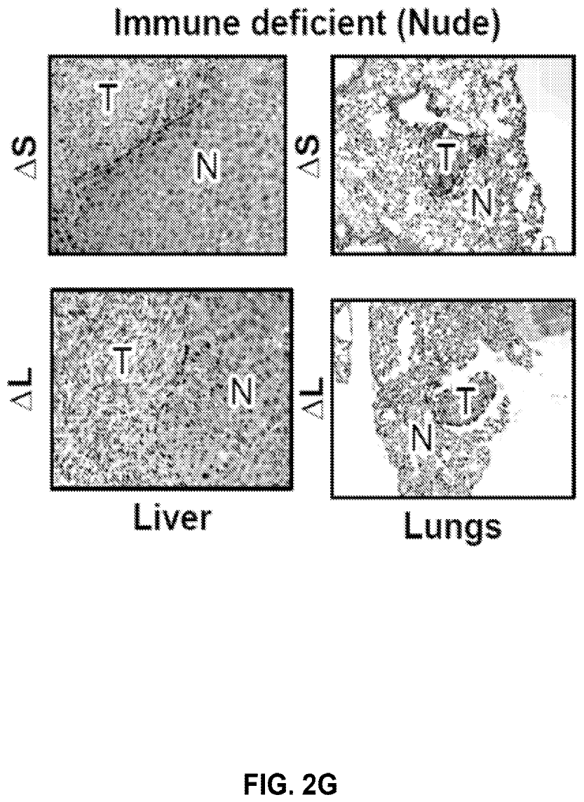

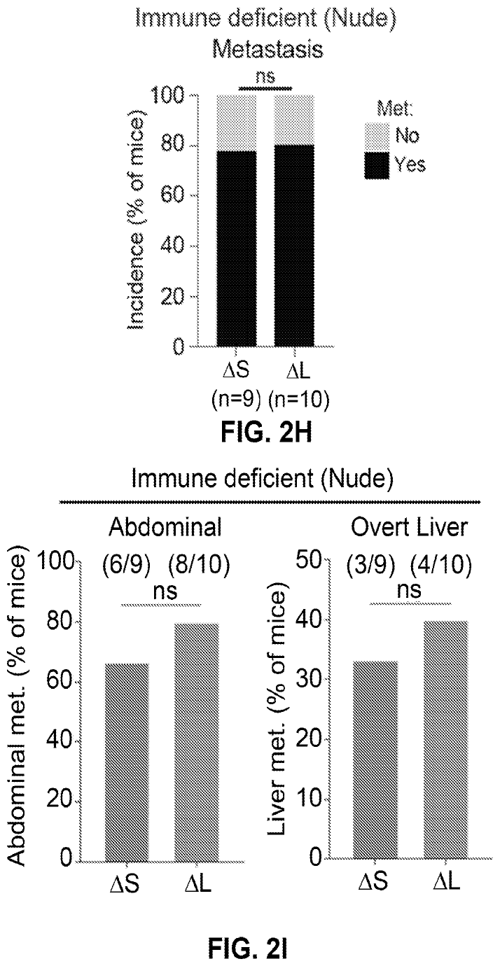

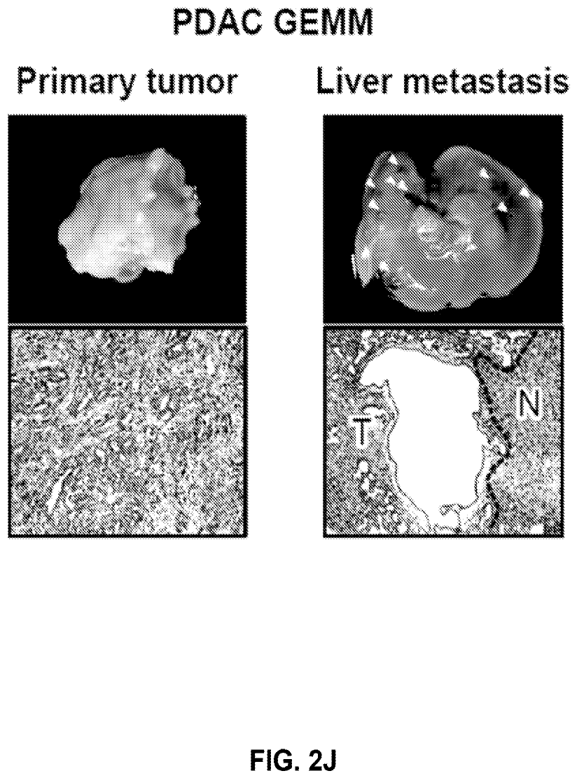

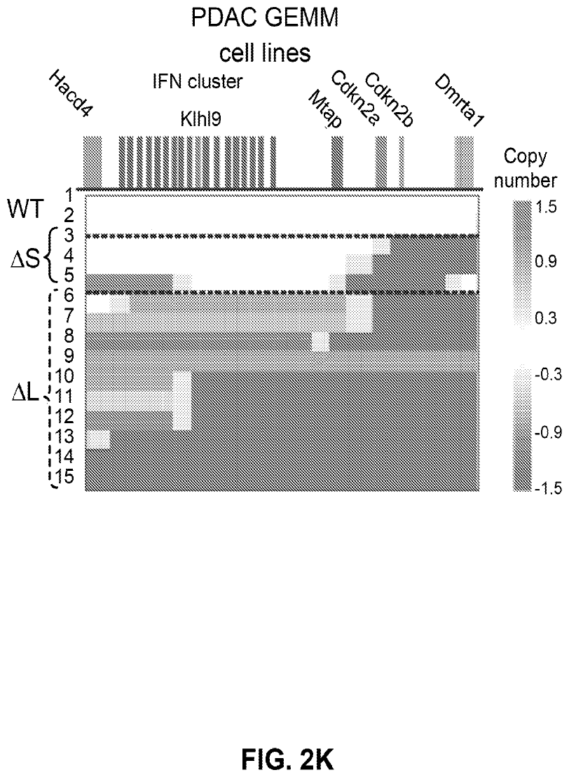

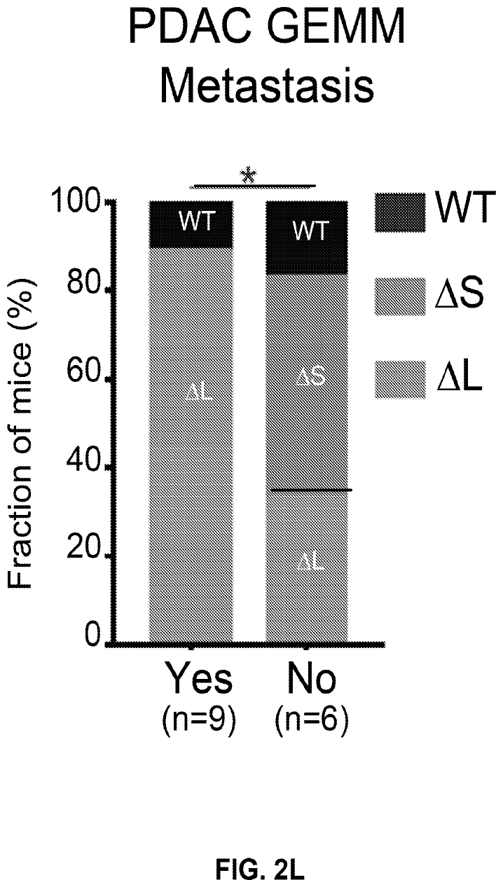

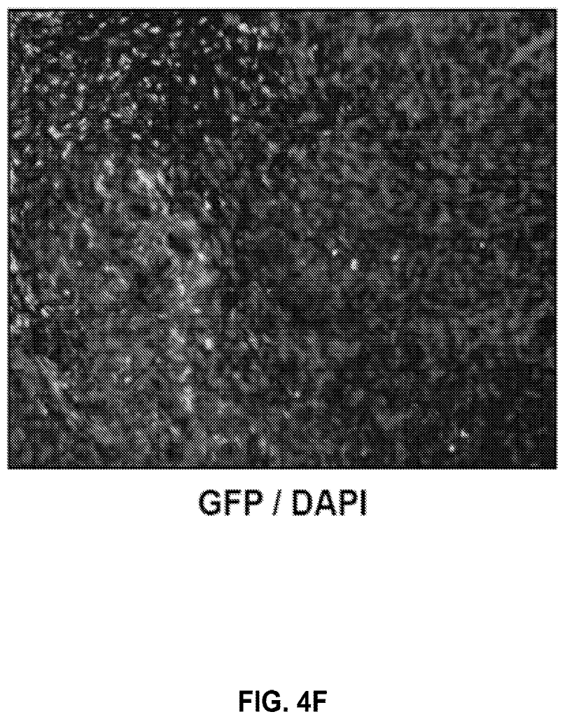

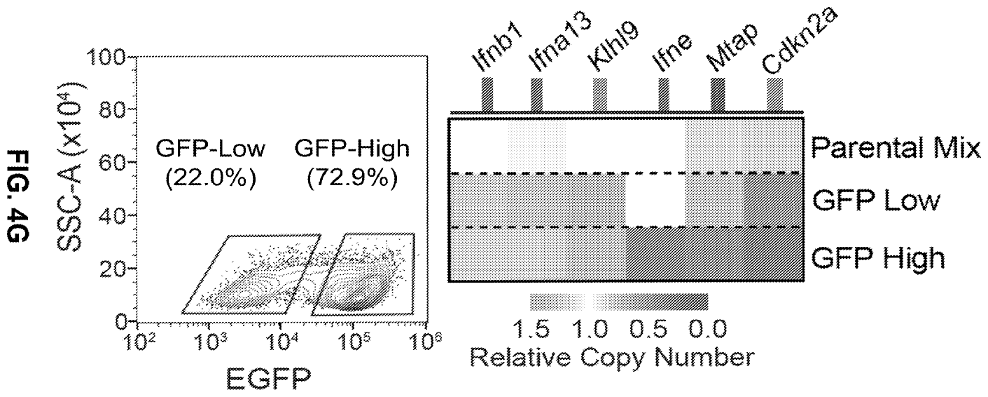

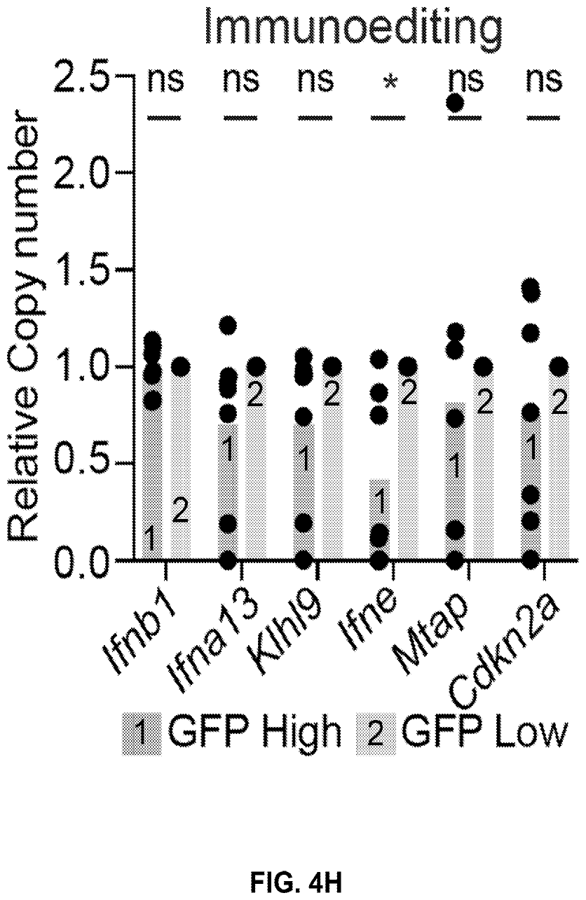

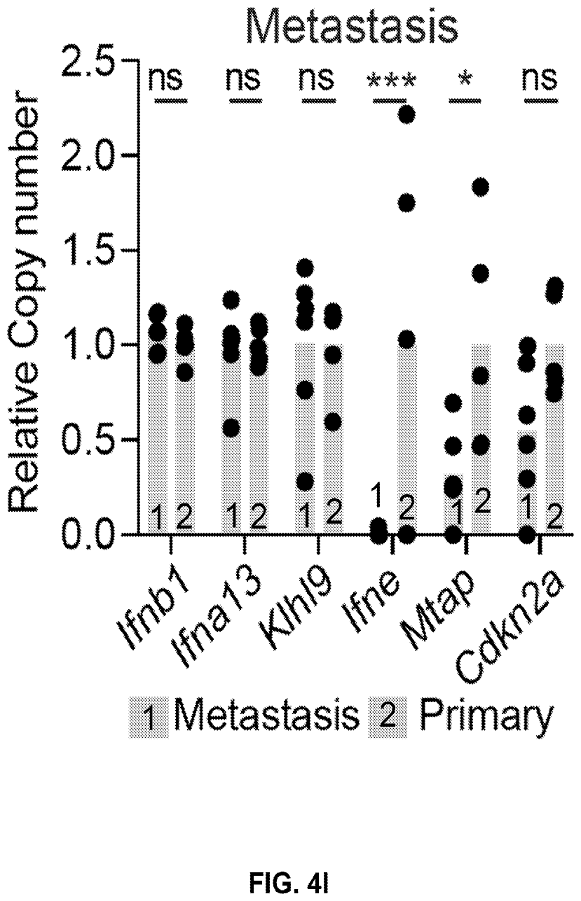

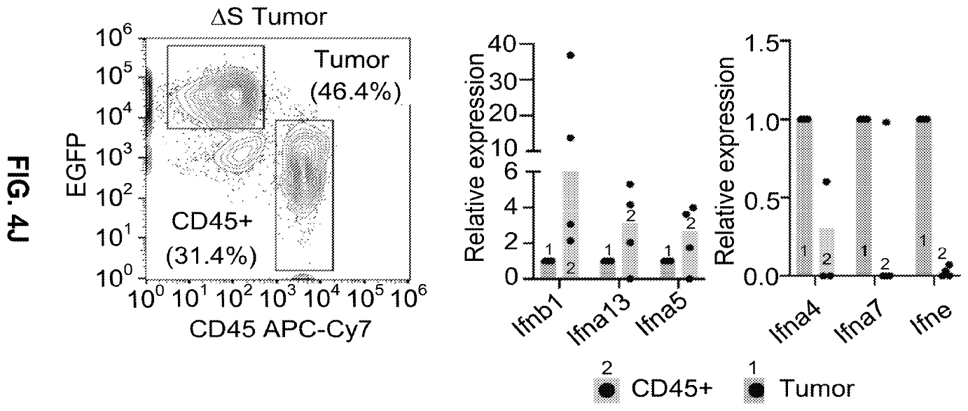

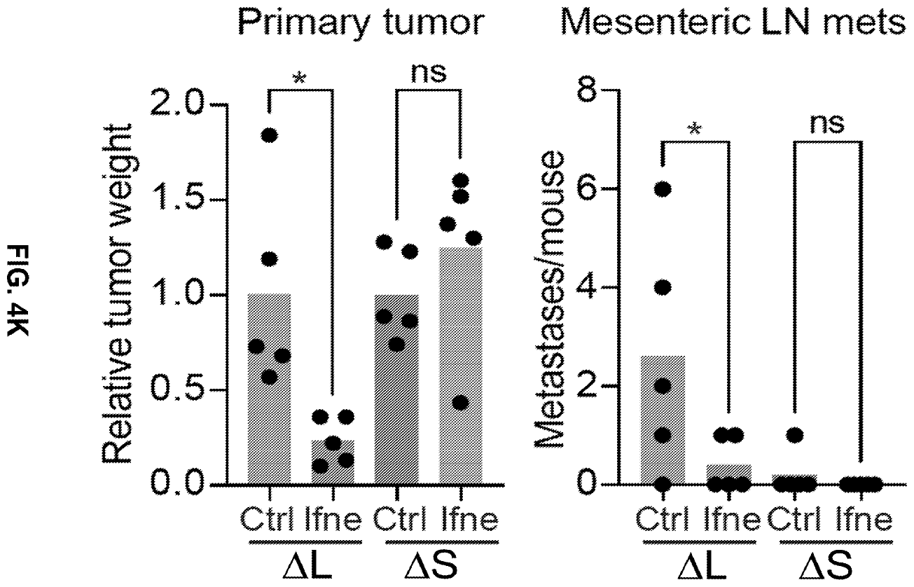

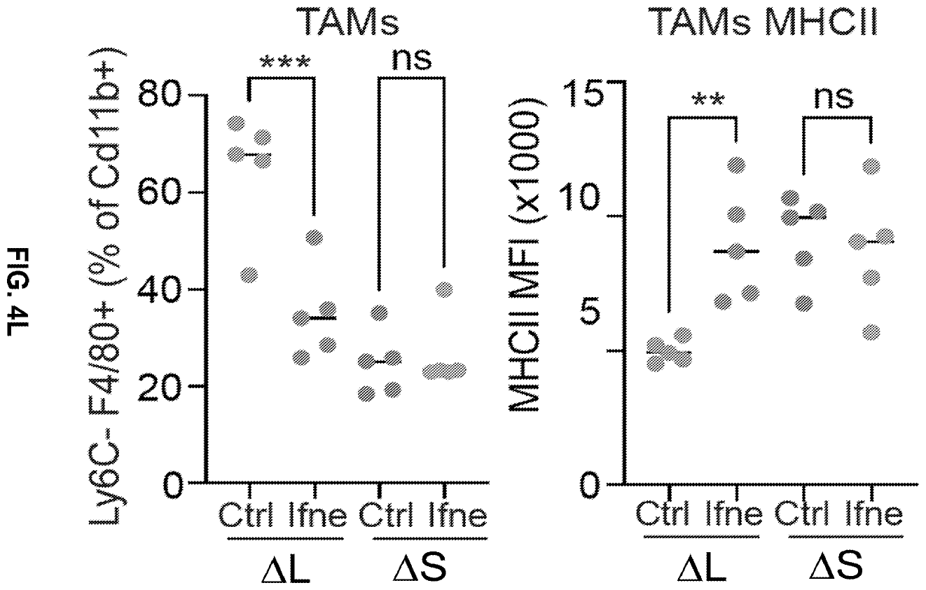

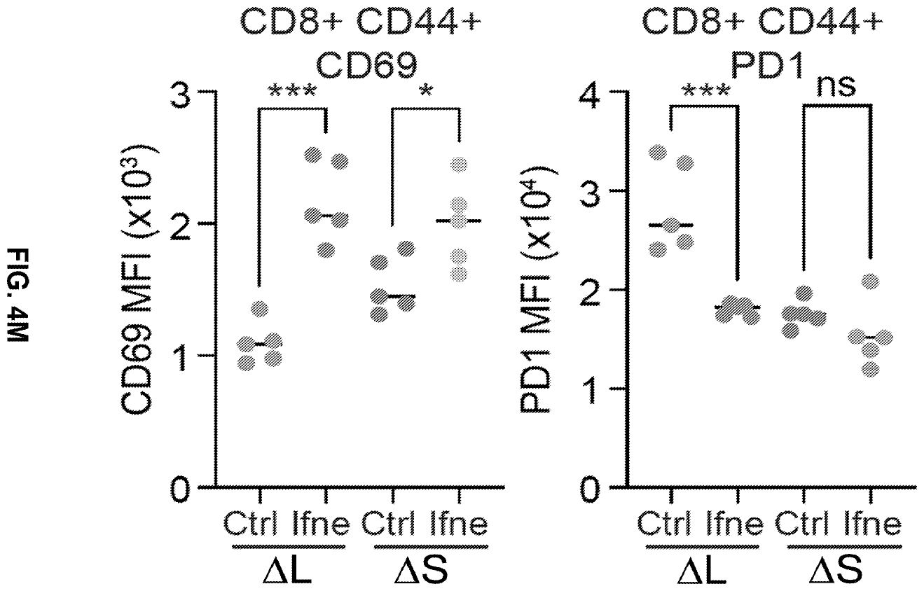

General Statistical Analysis. Graphs and statistical analyses for FIGS. 2, 4, 6-9, 11 and 12 were done with GraphPad Prism. For all experiments n represents the number of independent biological replicates. For FIGS. 2C, 6E, 8D, and 9A-91 differences were evaluated with a two-tailed t-test. For FIGS. 4A-B, 4H-41, 4J-4M, 7G, 7K, 11D, 11F, and 121-12J, differences were assessed by a one-way ANOVA followed by Tukey or Sidak's multiple comparison test. To assess differences in tumor initiation or metastasis incidence, contingency tables followed by a chi-square test were done for FIGS. 2A, 2E, 2F, 2H, 2I, 2L, 3K, 7F, and 7M. For survival curves, log rank-test was used to assess significant differences. Differences were considered significant for p values <0.05, where asterisks represent the level of significance for the analysis used: *, p<0.05; ** p<0.01; ***, p<0.001; n.s. not significant, p >0.05.

### Example 2: MACHETE Enables Efficient Generation of Megabase-Sized Genomic Deletions in Cellular Models

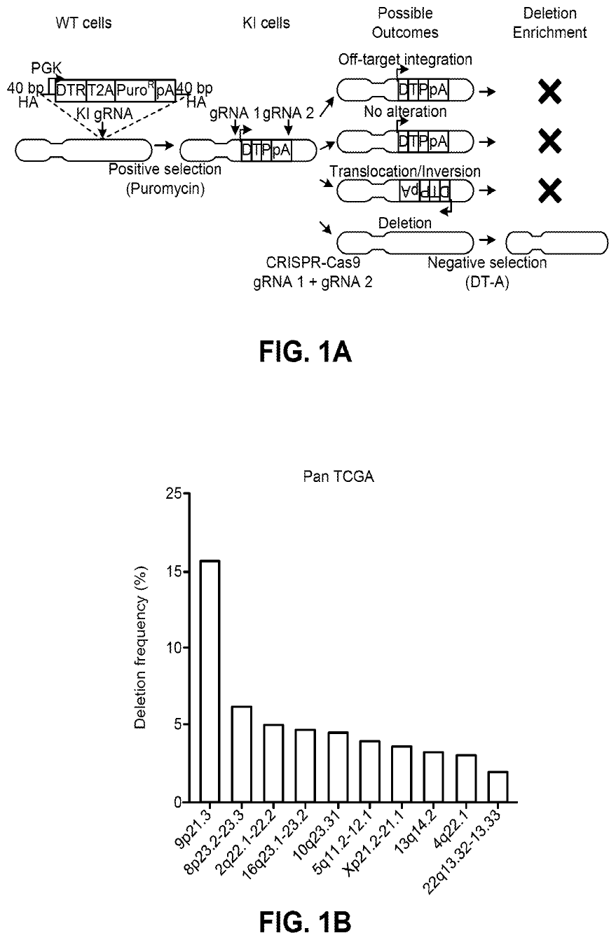

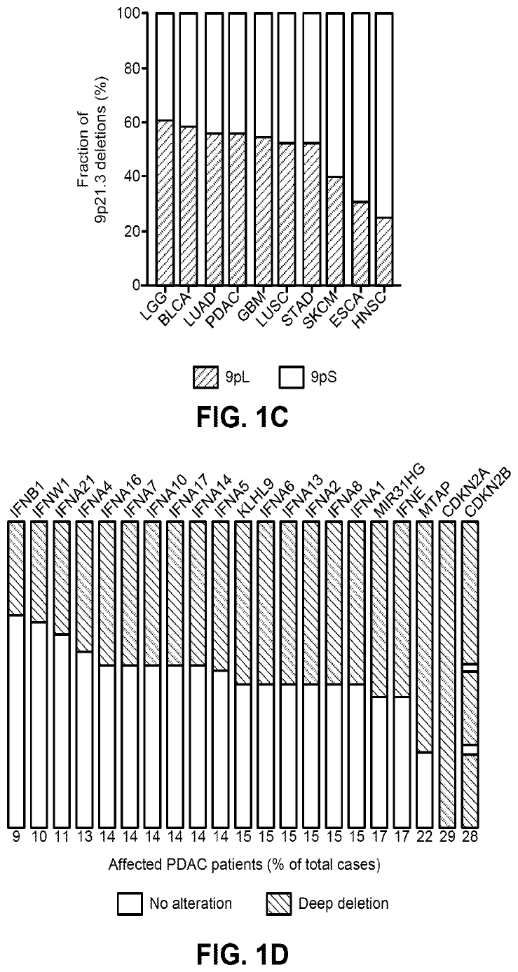

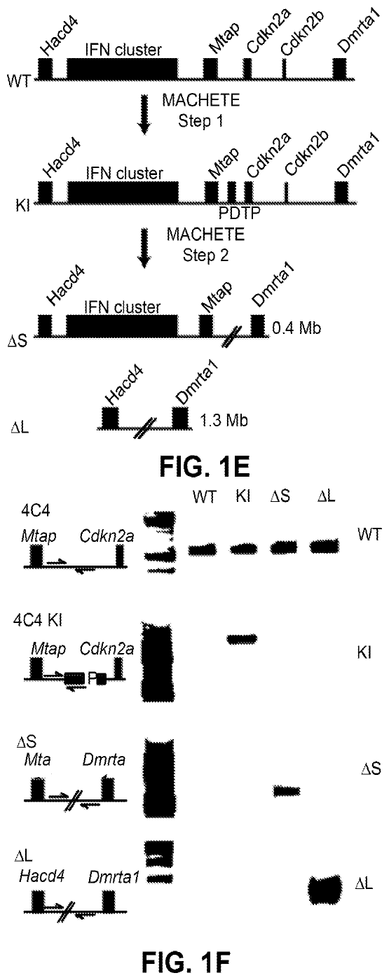

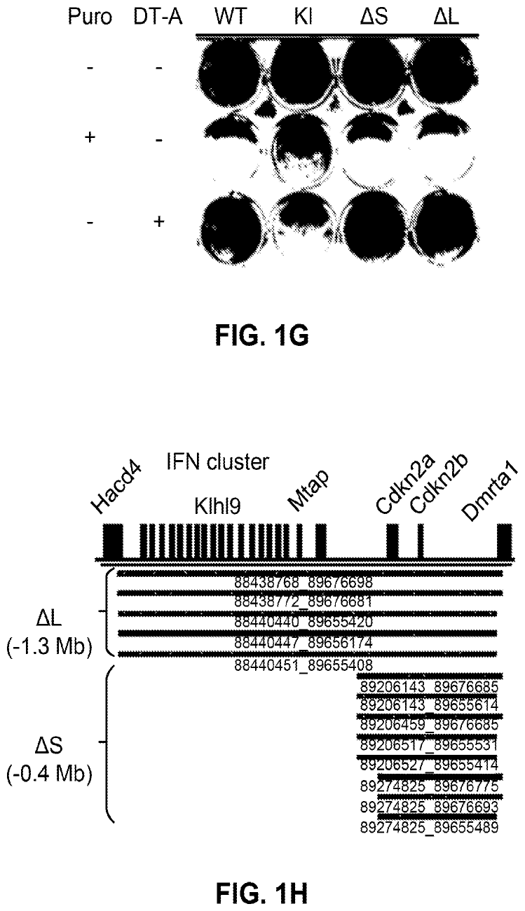

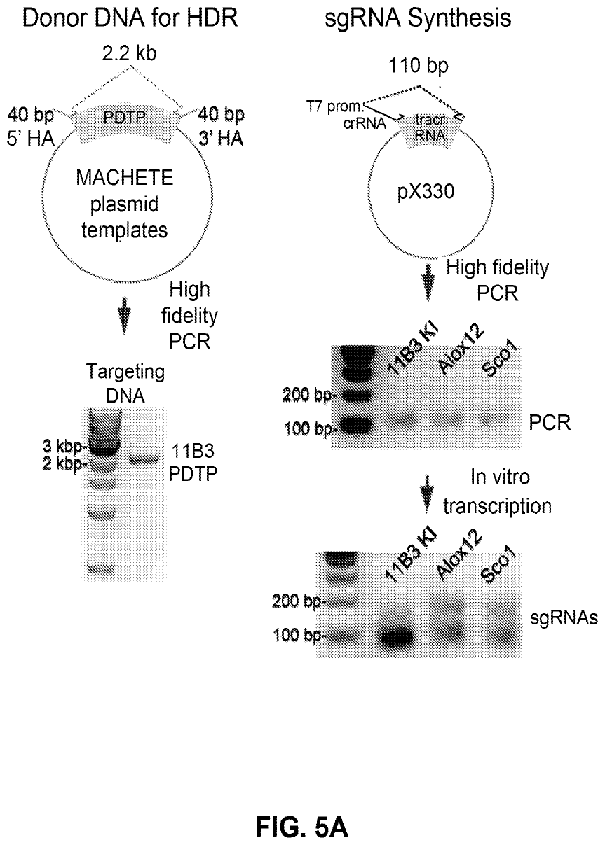

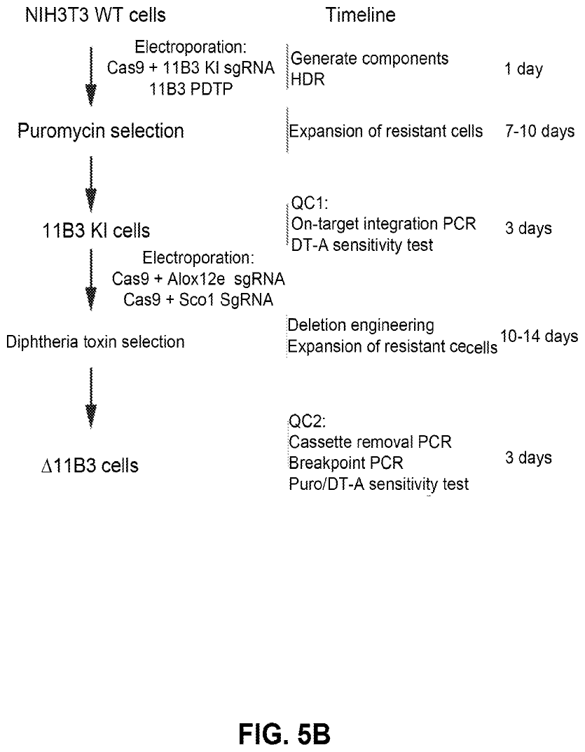

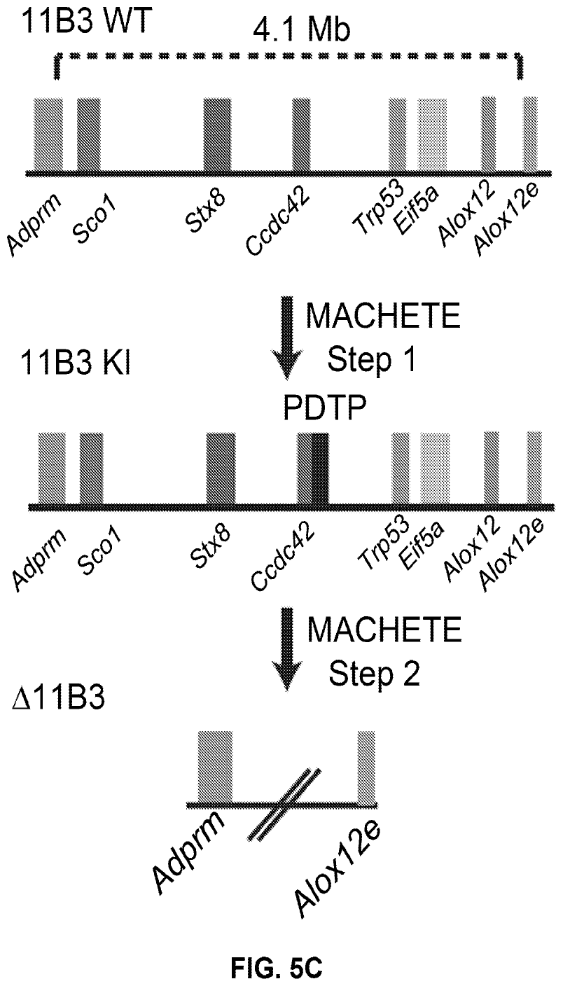

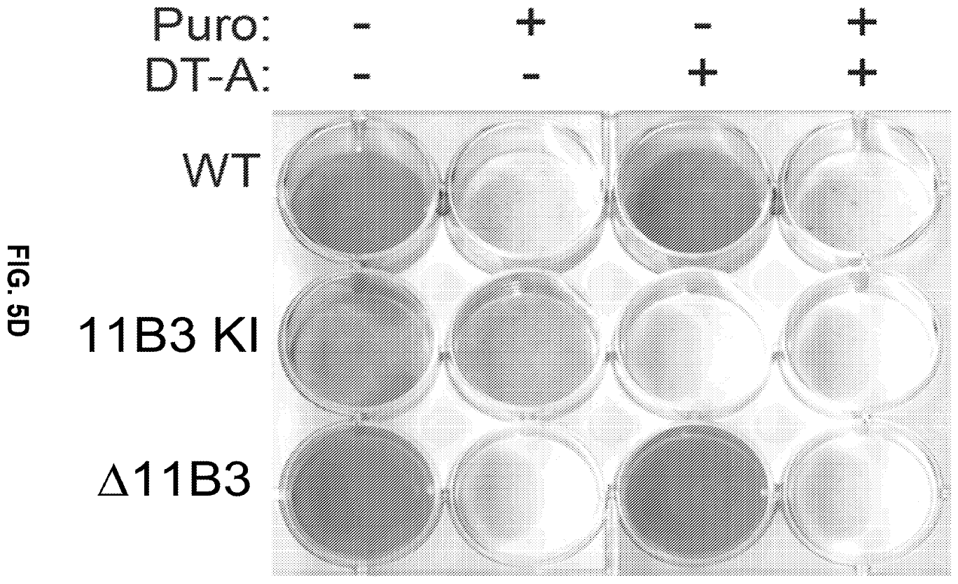

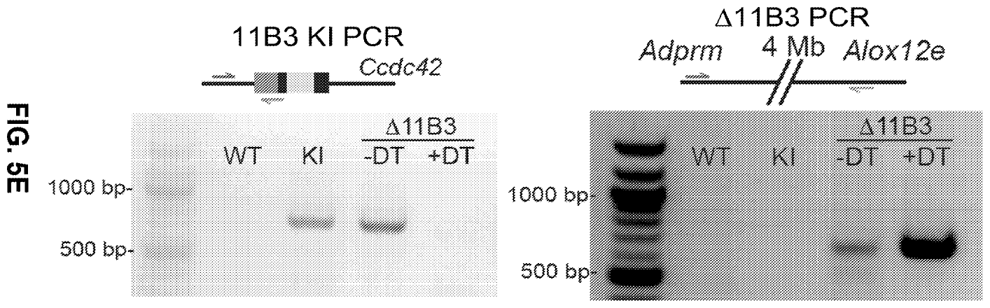

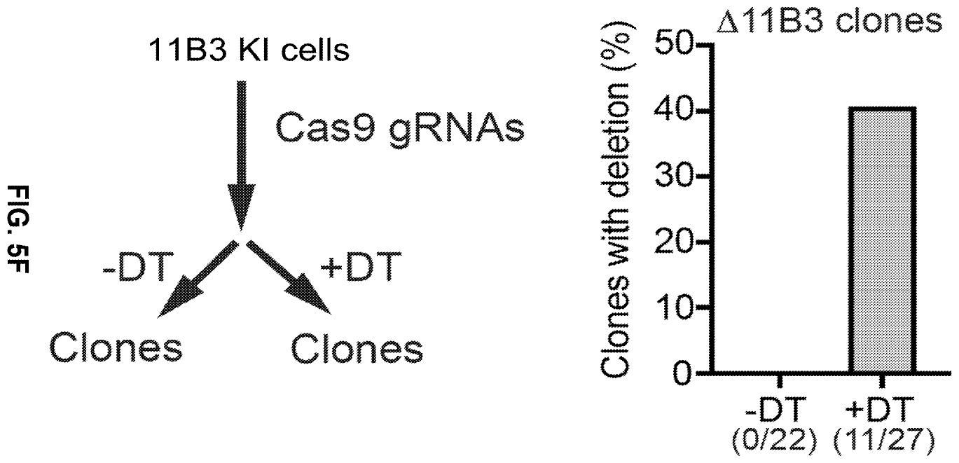

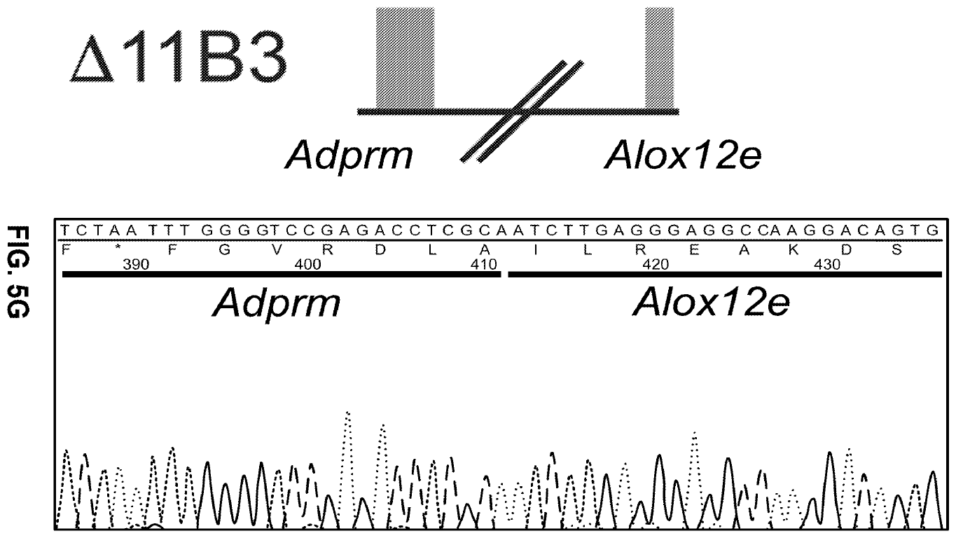

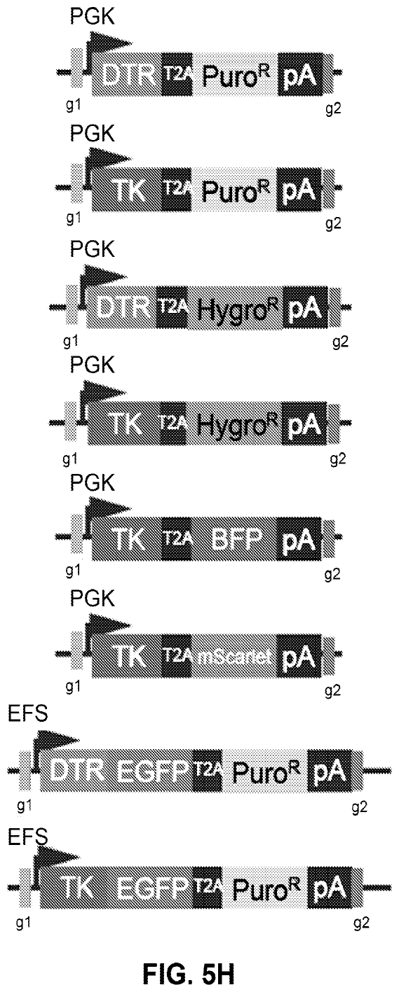

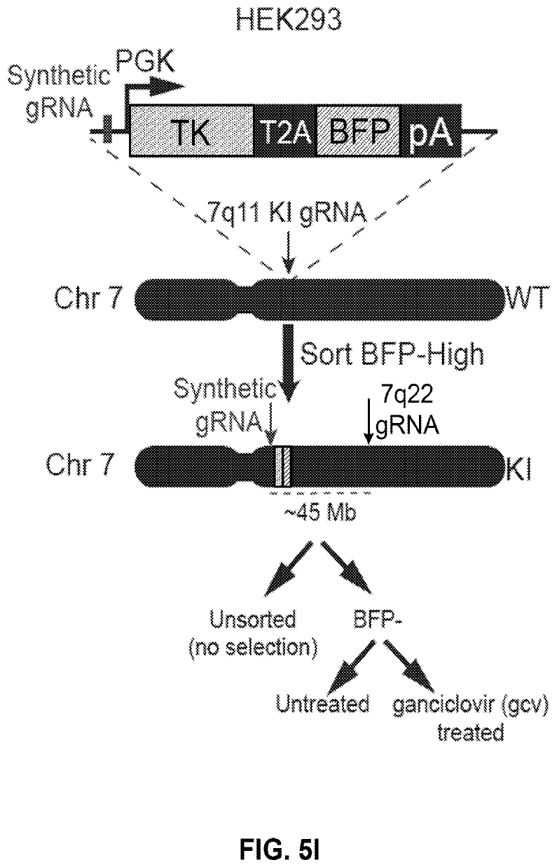

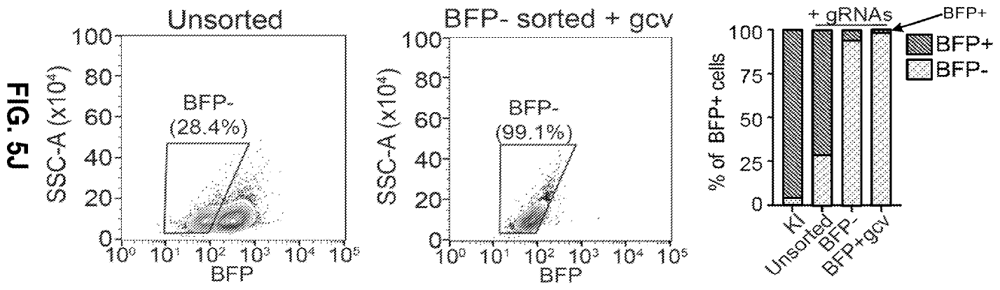

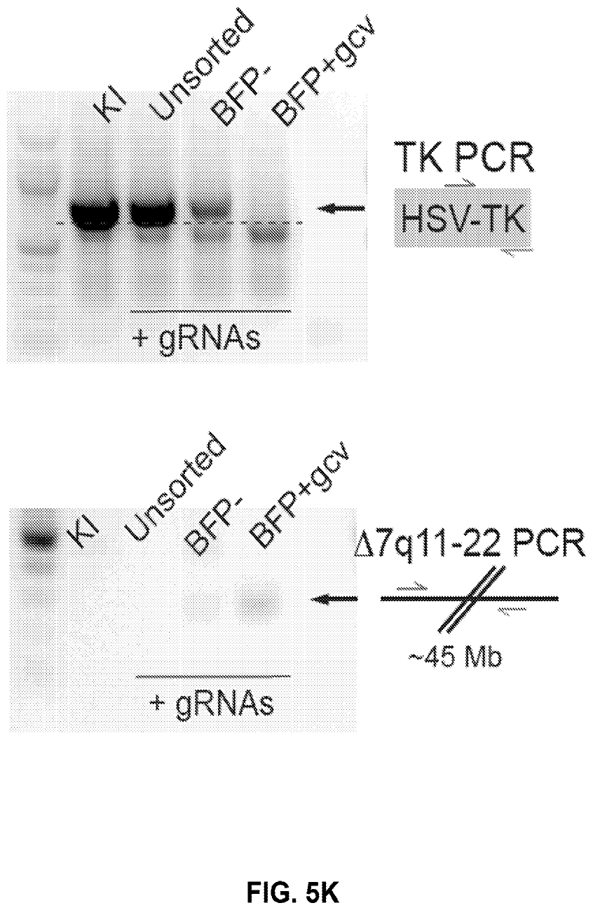

To facilitate the experimental study of genomic deletions, we developed a rapid and flexible approach to engineer megabase-sized deletions termed Molecular Alteration of Chromosomes with Engineered Tandem Elements (MACHETE). MACHETE involves an integrated process that inserts a selection cassette within a region of interest, followed by its co-deletion with defined regions of flanking DNA (FIG. 1A). First, a bicistronic cassette encoding tandem negative and positive selection markers is amplified using oligos with homology to a region within an intended deletion. Second, the cassette is then inserted into the genome by CRISPR-facilitated homology-directed repair, and cells with integrations are enriched by positive selection. Third, a pair of single guide RNAs (sgRNAs) targeting the breakpoints of the intended deletion are introduced on either side of the bicistronic cassette, followed by negative selection. Since the sequence specificity of the flanking guides exclusively deletes on-target integrations of the suicide cassette, the latter step not only eliminates cells that retain the selection cassette but also those harboring off-target integrations (FIG. 1A). Notably, the MACHETE protocol was designed to eliminate the need for cloning components: donor DNA is generated by introducing 40-bp homology arms via PCR amplification of the selection cassette, which is coupled to ribonucleoproteins (RNPs) of recombinant Cas9 complexed with sgRNAs (FIGS. 5A-5B). We envisioned that this approach would enable engineering of an allelic series of deletions, thereby enabling the systematic functional dissection of distinct regions within a locus.

As an initial proof of concept, we engineered a 4.1-Mb deletion of the murine 11B3 locus (syntenic to human 17p13.1), which encompasses the Trp53 TSG (FIG. 5C) and had been previously produced using a Cre/loxP approach¹³. NIH3T3 fibroblasts were targeted with a PGK-DTR-T2A-Puro (PDTP) dual-selection cassette to an intronic region of Ccdc42, a gene located in the 11B3 locus, and positively selected for insertion of the cassette (11B3 knock-in (KI) cells). Cas9-sgRNA RNPs were then introduced to target regions flanking Scol and Alox12, the genes that demarcate the intended deletion, and negative selection was performed using DT to produce a cell population termed D11B3 (FIG. 5C). Parental, 11B3 KI, and D11B3 populations showed the expected pattern of resistance or sensitivity to the selection agents, presence/absence of the cassette, and expected deletion breakpoint (5D-5E). Clonal analysis showed that use of negative selection effectively enabled the generation of the desired deletion, by increasing the efficiency of D11B3 engineering from undetectable (0/22) to 40% of positive clones (11/27, all heterozygous) (FIG. 5F), which was confirmed by sequencing (FIG. 5G).

We further developed a series of constructs that enable the use of MACHETE across various experimental contexts (FIG. 5H). To illustrate the use of MACHETE in human cells, we selected a cassette composed by a herpes simplex virus thymidine kinase with blue fluorescent protein (HSV-TK-T2A-BFP), which enables positive selection via fluorescence activated cell sorting (FACS) and negative selection using ganciclovir. This construct enabled the production of cells harboring a 45 Mb deletion of chromosome 7q11-7q22 (FIGS. 51-5K). Thus, MACHETE is a customizable approach to efficiently engineer large chromosomal deletion events.

### Example 3: Loss of Type I IFN Genes is a Common Event in 9p21.3 Deleted Tumors

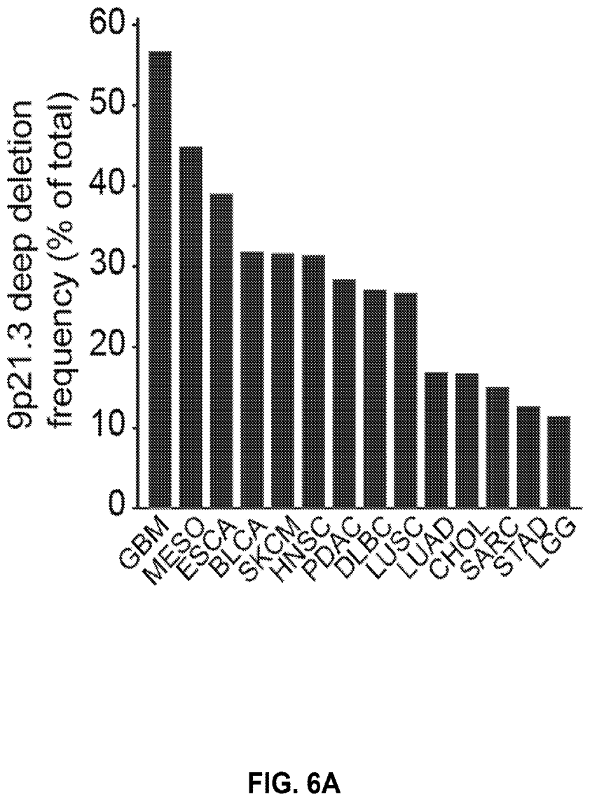

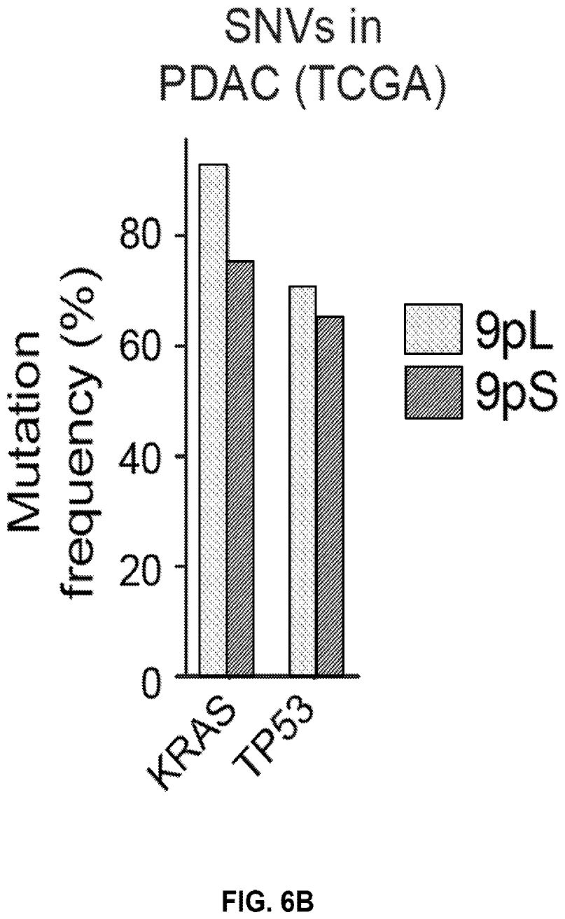

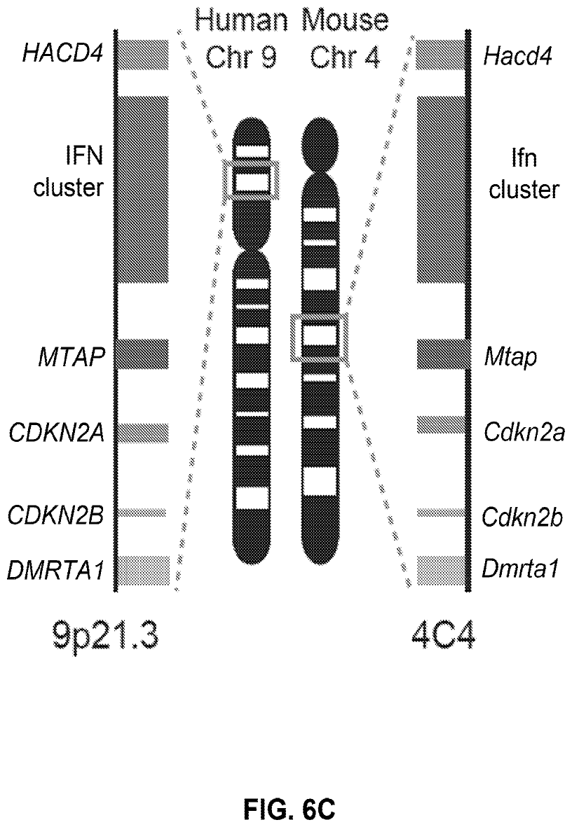

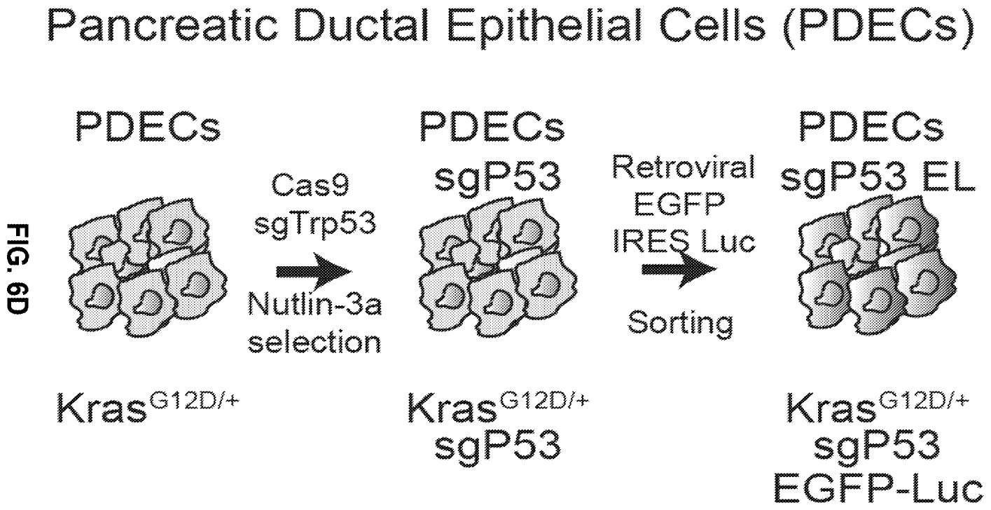

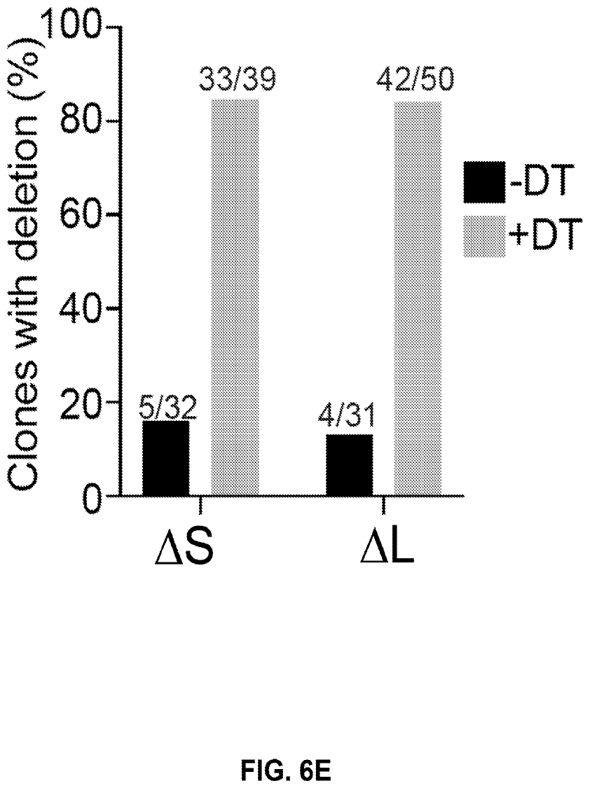

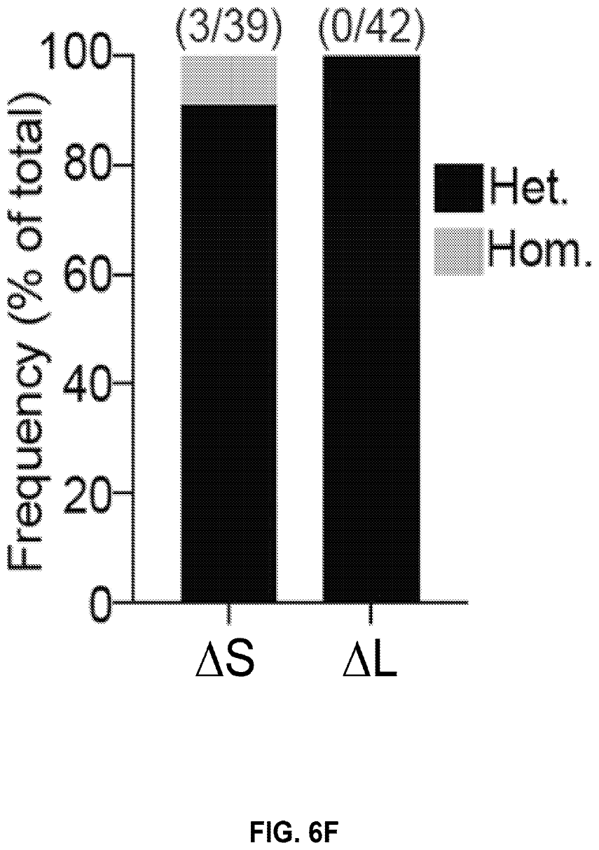

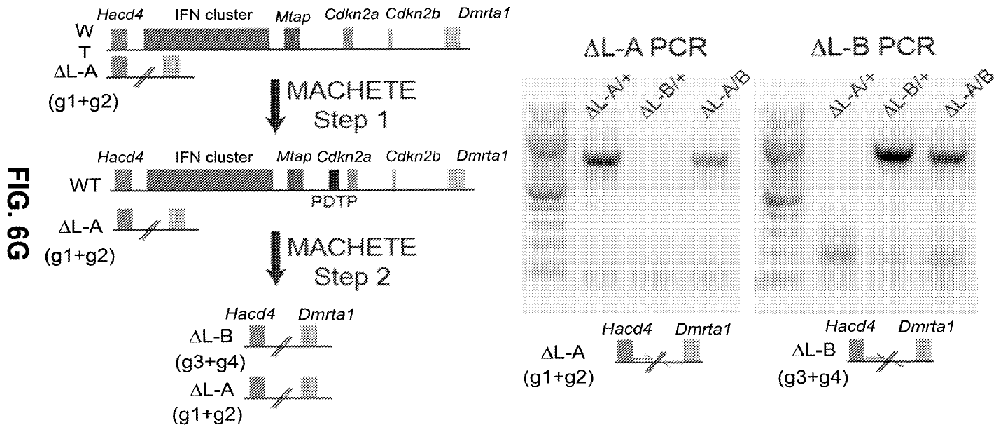

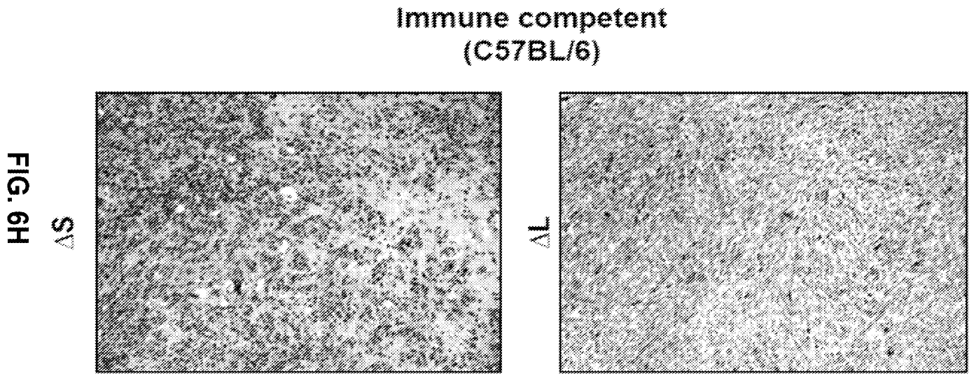

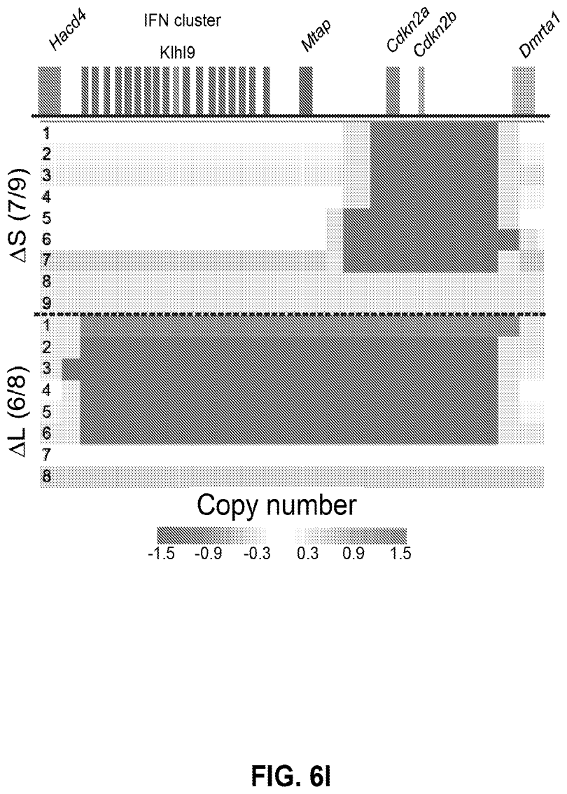

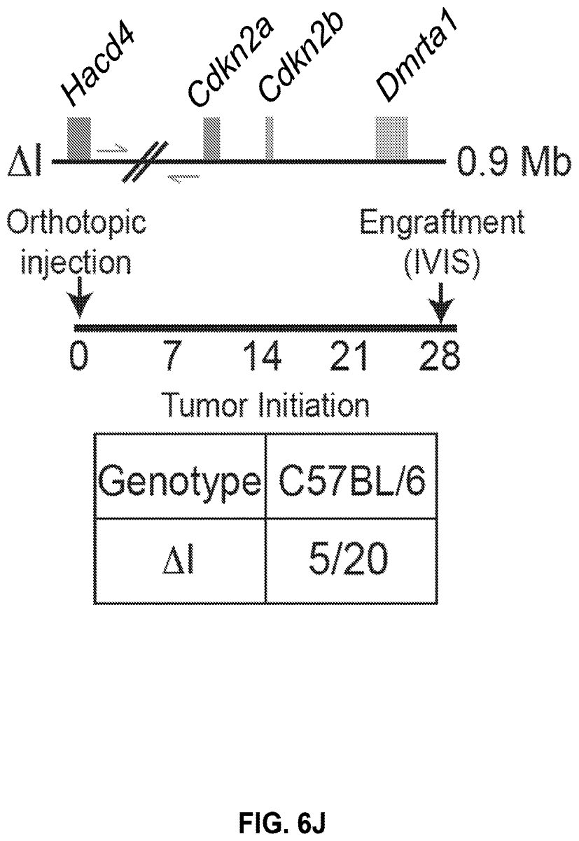

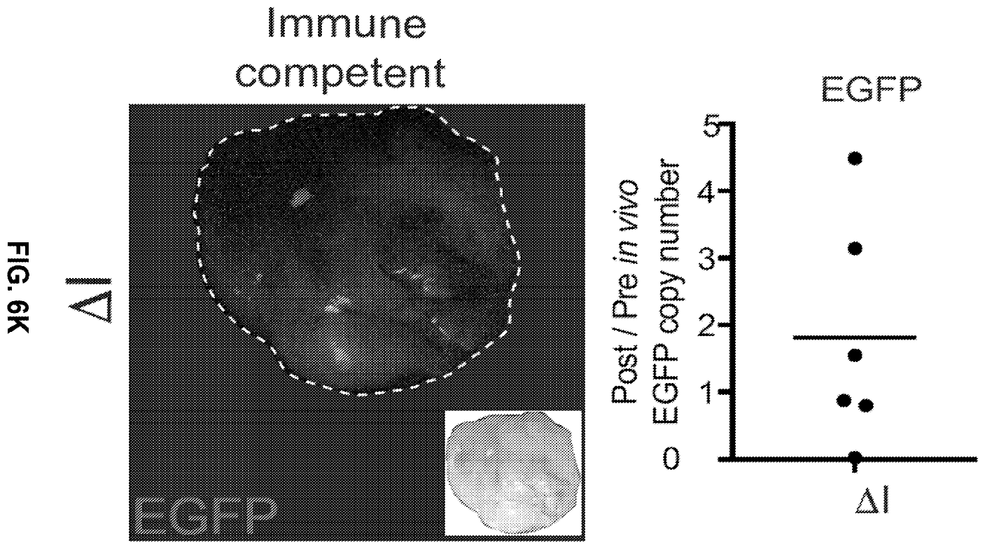

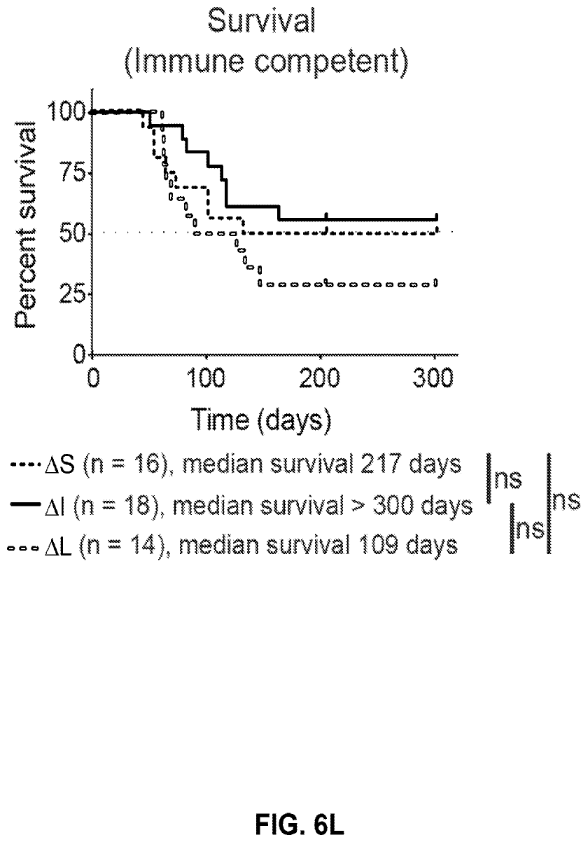

Armed with MACHETE, we set out to interrogate the biology of deletions at the 9p21.3 locus (FIG. 1B). Interestingly, although (DKN2A is a well-established tumor suppressor in this region, we and others have noted that 9p21.3 deletions can encompass additional genes, including a cluster of 16 type I IFN genes whose genetic loss has not been functionally implicated in tumorigenesis despite the known role of IFN signaling in anti-tumor immunity³⁰. An analysis of the TCGA dataset³¹ revealed that fourteen different tumor types harbor homozygous 9p21.3 deletions in over 10% of cases (FIG. 6A). We further classified 9p21.3 deletions into those targeting CDKN2A/B alone (9p small, or 9pS) or larger events that typically encompassed the entire type I IFN cluster (9p large, or 9 pL) (FIG. 1C). The frequency of the 9 pL events ranged between 20-60% depending on tumor type and was one of the highest in pancreatic ductal adenocarcinoma (PDAC) (FIG. 1D).

### Example 4: Engineering 9p21.3 Deletions in Mouse Models of PDAC

Genetic analyses of human PDAC indicate that (DKN2A deletions are an early event in tumor evolution^{32,33}, which are thought to emerge as heterozygous deletions that subsequently undergo loss of heterozygosity^{34,35}. These deletions tend to co-occur with activating KRΔS mutations and TP53 loss, two other major drivers in this disease (FIG. 6B)³⁶. Given the potential role of type I IFNs in modulating immune responses, we set out to study the biology of different 9p deletions in a syngeneic model of murine PDAC derived from established pancreatic ductal epithelial cells (PDECs) that harbor an endogenous activated Kras^{G12D} allele^{37,38}. While Cdkn2a expression is blunted in this system, the lesions produced following PDEC transplantation resemble premalignant stages of PDAC, display a limited capacity to progress to invasive adenocarcinoma³⁸, and allow the study of immune-related processes^{37,39}. Thus, given the synteny between human 9p21.3 and murine 4C4 (FIG. 6C), PDEC cells provide a good platform for MACHETE-based engineering of 9p21.3 equivalent deletions in vitro and the subsequent study of tumor phenotypes in an immune competent in vivo context.

To model the most relevant genetic configuration for 9p21.3 loss in human PDAC, we generated Trp53 knockout PDEC cells using transient CRISPR-Cas9 and introduced an EGFP-Luciferase cassette to enable visualization of engrafted cells (PDEC-sgP53-EL cells) (FIG. 6D). MACHETE was then used to engineer the two most frequent configurations of 9p21.3 deletions: ΔS (“Small”; 0.4 Mb loss spanning Cdkn2a and Cdkn2b), and ΔL (“Large”; 1.3 Mb loss spanning the entire 4C4 locus) (FIGS. 1E-1G). Deep sequencing of the breakpoint regions confirmed the presence of precise 0.4 and 1.3 Mb deletions, and clonal analysis of targeted cell populations indicated that MACHETE achieved an >8-fold increase in producing cells with the intended heterozygous deletion (FIG. 1H, FIGS. 6E-6F). As expected, these populations could be further edited through MACHETE's capability for iterative engineering (FIG. 6G). Given the comparable deletion efficiency of ΔS and ΔL cells, cell populations were used for subsequent analyses to minimize the effects of clonal variation.

### Example 5: Tumors with ΔL Deletions are Differentially Surveilled by the Adaptive Immune System

To determine whether each heterozygous deletion event contributes to tumorigenesis, we transplanted the ΔS and ΔL lines into the pancreata of syngeneic C57BL/6 recipients and assessed tumor formation at 4 weeks via bioluminescent imaging and at endpoint. Cells bearing the ΔL deletion tended to form more tumors than ΔS cells, although the difference was not statistically significant (FIG. 2A). Tumors arising from both genotypes were poorly differentiated, consistent with the histopathology of autochthonous Trp53- and Cdkn2a-deficient PDAC models (FIG. 6H) 40. Sparse whole genome sequencing (sWGS) confirmed that most ΔS or ΔL tumors acquired homozygous deletions of their respective alleles (7/9 lines for AS; 6/8 lines for ΔL), as occurs in human PDAC (FIG. 6I). However, there was one notable difference: ΔL tumors retained a strong EGFP fluorescence signal and genomic copy number compared to ΔS tumors (FIGS. 2B-2C).

The above findings are consistent with immunoediting of cells with high reporter expression⁴¹ and raise the possibility that ΔL cells may be less immunogenic than their AS counterparts. Accordingly, ΔS and ΔL cells showed a similar capability of forming EGFP-expressing tumors in Foxn1^{nu} (“nude”: T and B cell deficient) and NOD/SCID Il2rg^{-/-} (NSG: T, B, and NK cell deficient) mice (FIGS. 2A-2C). Interestingly, cell populations engineered to harbor 4C4 deletions that eliminate upstream elements but that retained the Cdkn2a b genes (AI allele) had reduced tumor initiating capacity yet produced tumors that expressed similar levels of EGFP as ΔL tumors (FIGS. 6J-6L). These data imply that genetic elements upstream of Cdkn2a b contribute to immunoediting of developing tumors.

### Example 6: ΔL Deletions Promote Metastasis by Evasion of Adaptive Immunity

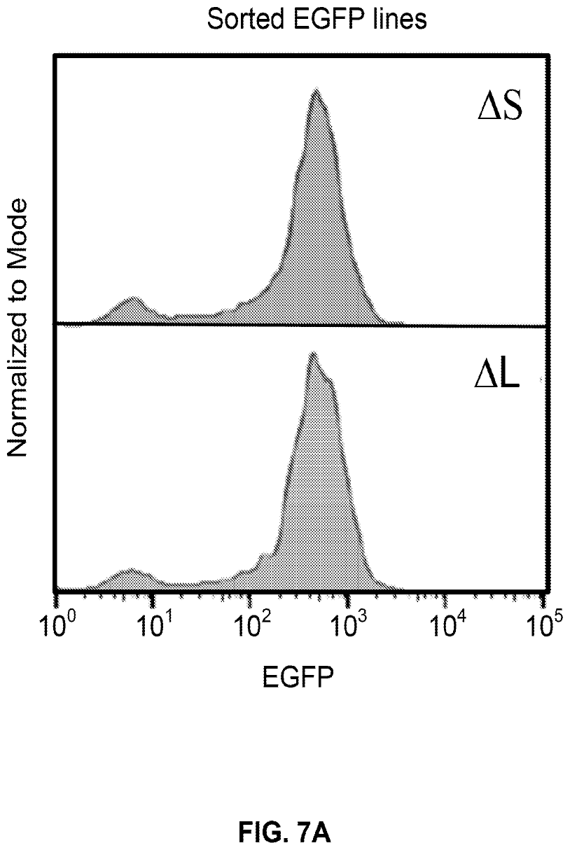

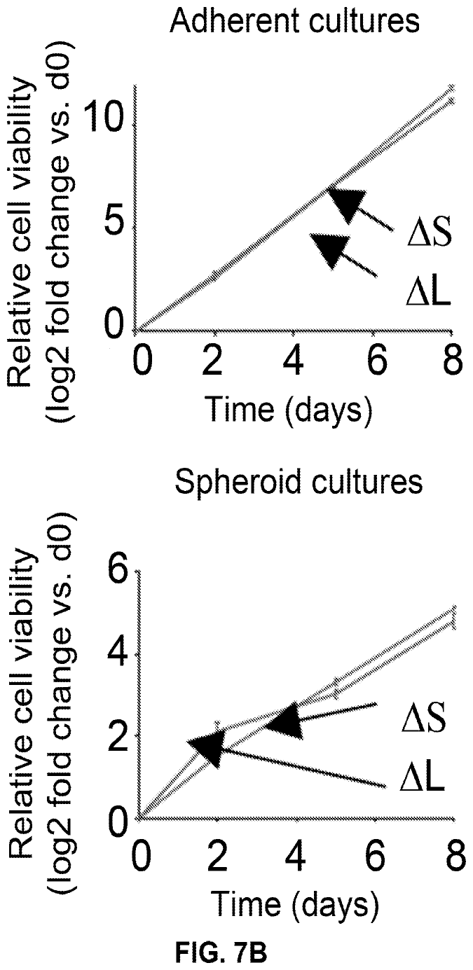

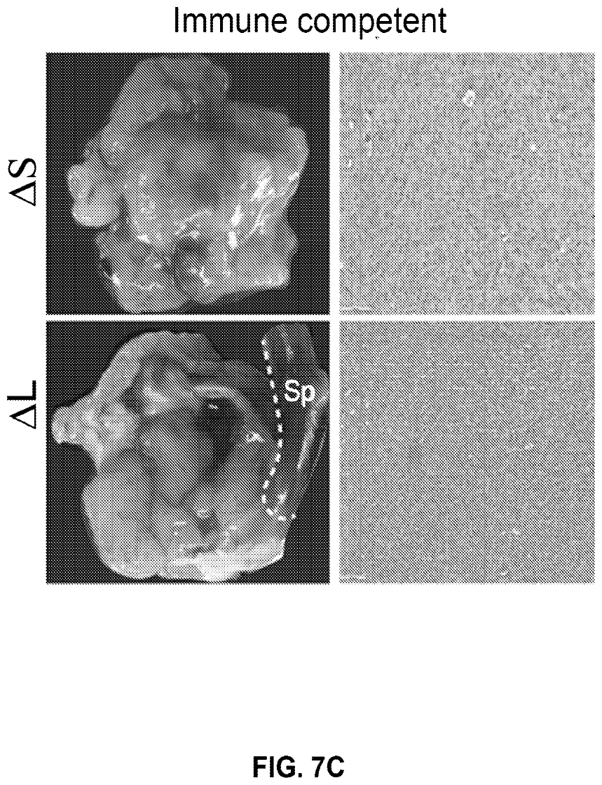

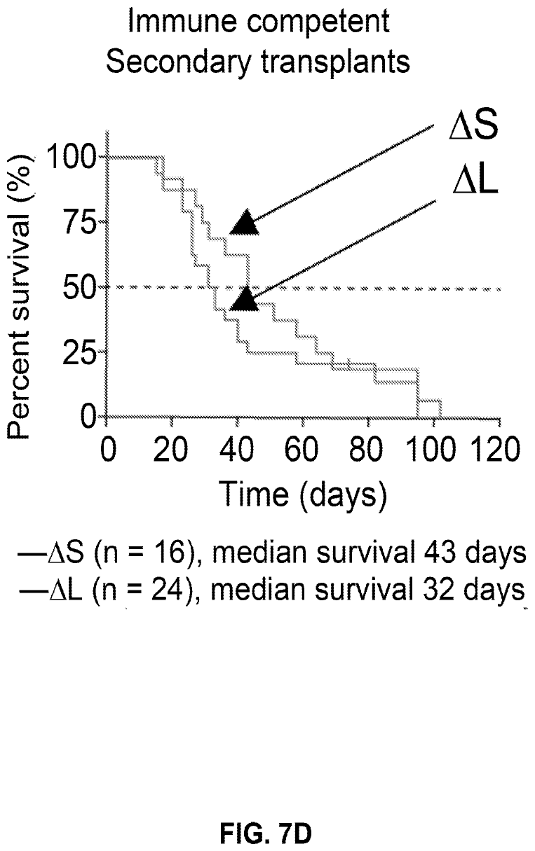

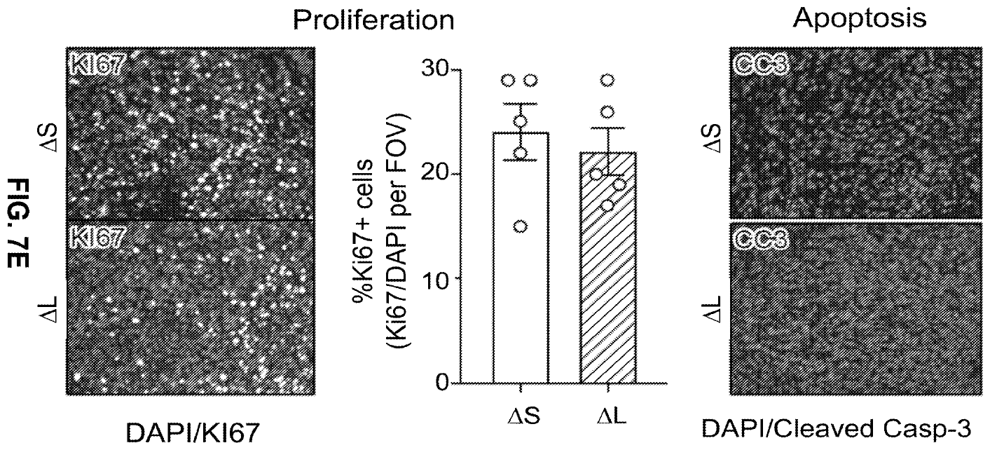

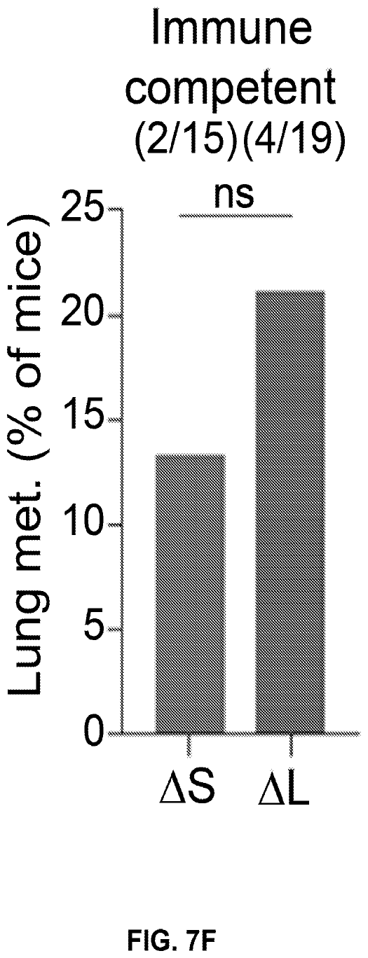

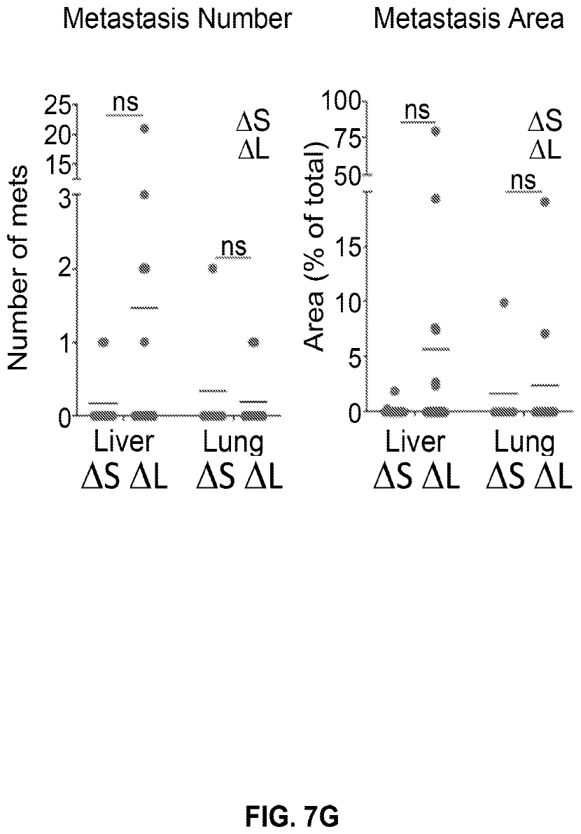

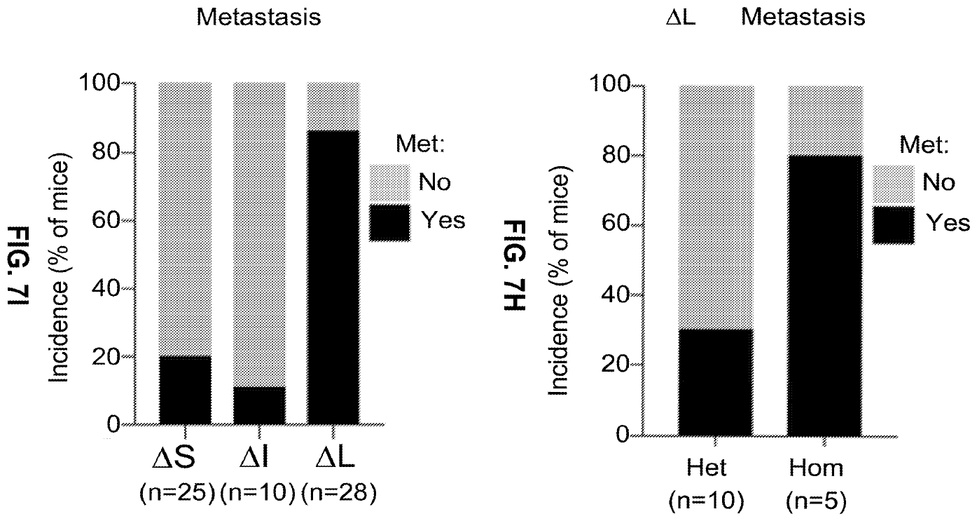

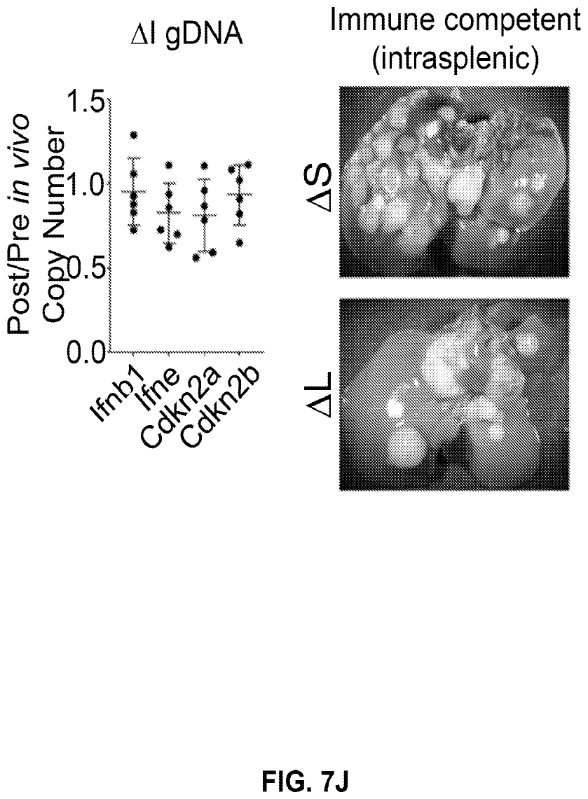

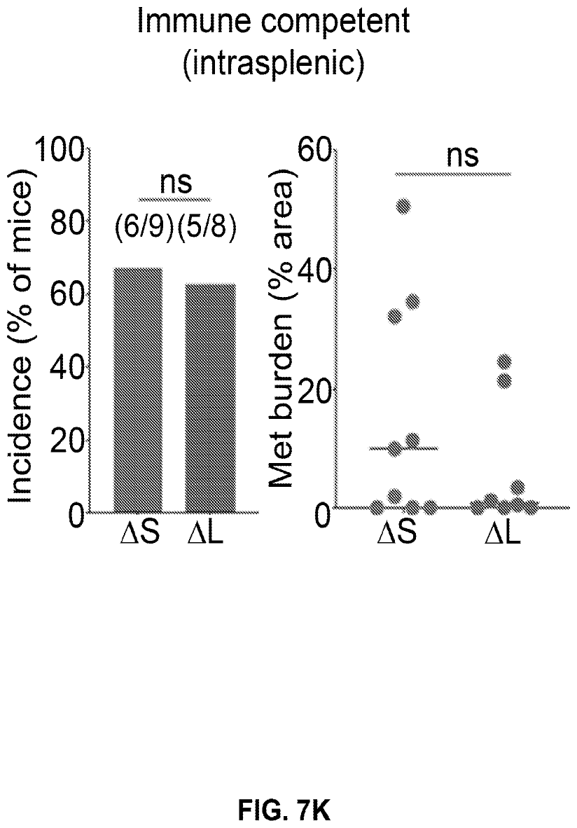

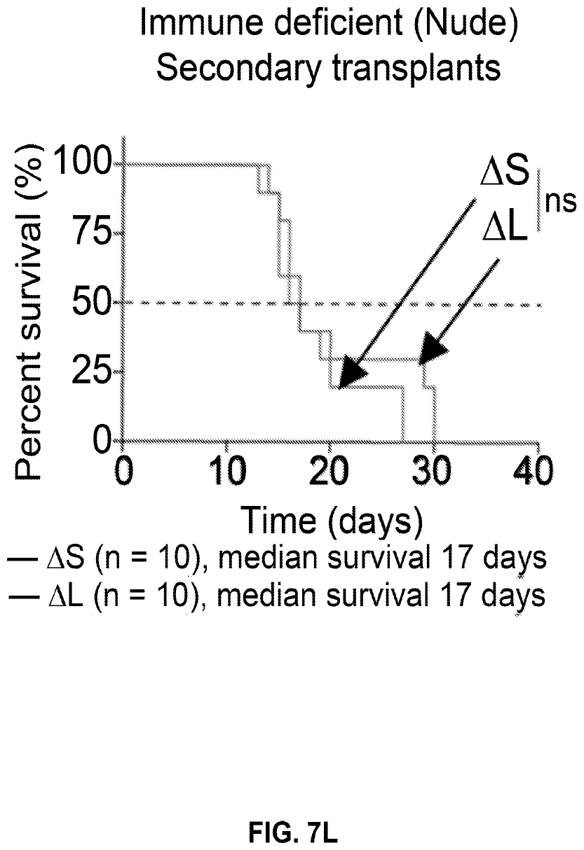

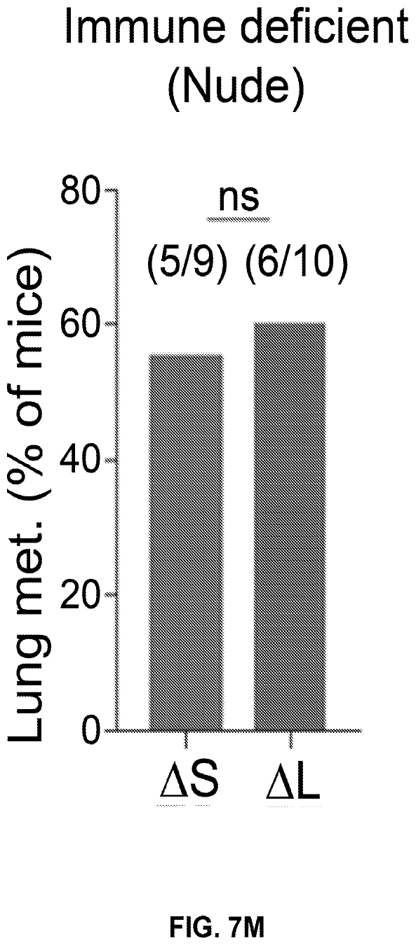

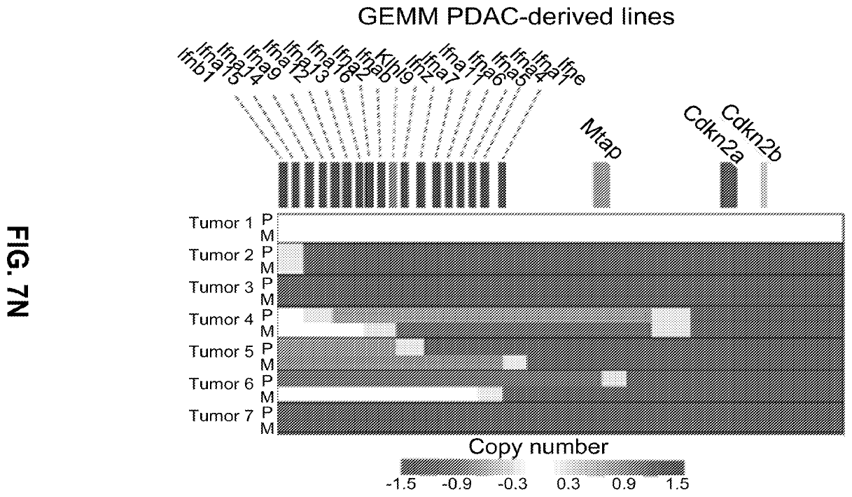

We next compared the behavior of ΔS and ΔL tumor-derived cell lines in orthotopic transplantation assays. Four independently derived ΔS and ΔL tumor lines were FACS-sorted to obtain cell populations with comparable EGFP levels to eliminate differences in reporter expression as a confounding factor (FIG. 7A). ΔS and ΔL tumor cells showed a similar ability to proliferate in adherent or suspension cultures and produced tumors with undifferentiated histopathology (FIGS. 7B-7C). However, consistent with their acquisition of homozygous 4C4 deletions, the tumors progressed more rapidly (FIG. 7D).

Although ΔS and ΔL tumors showed no obvious difference in the fraction of proliferating or apoptotic cells (FIG. 7E), ΔL tumors were much more prone to metastasis (FIG. 2D-2E). Indeed, these mice displayed a 4-fold higher incidence of macrometastases in the abdominal area (mesenteric lymph nodes, intestine, and peritoneal cavity) compared to their ΔS counterparts, and uniquely harbored overt liver metastases (˜25% of mice) (FIG. 2F). These observations were confirmed through histological analyses, which also indicated a trend for larger number and area of liver lesions (FIGS. 7F-7G).

Further insights into the greater metastatic potential of ΔL cells were obtained through analyzing additional tumor genotypes and routes of cell delivery, or by studying tumor behavior in immunocompromised animals. First, tumor-derived cells that remained heterozygous for the ΔL (2/8 lines that did not undergo LOH) or ΔI alleles were unable to efficiently produce metastases following orthotopic injection (FIGS. 7H-7I). Second, homozygous ΔS or ΔL tumor cells were equally able to produce experimental liver metastases following intrasplenic injection (FIGS. 7J-7K). Third, as was observed for the immunoediting phenotype, homozygous ΔS and ΔL cells showed a similarly high frequency of metastasis following orthotopic injection into Nude mice (FIGS. 2G-21, FIGS. 7L-7M). Therefore, the enhanced metastatic propensity of ΔL cells requires concomitant homozygous deletion of Cdkn2a b and the IFN cluster and involves an immune surveillance mechanism that acts prior to the colonization at distant sites.

### Example 7: Loss of Type I IFNs Correlates with Metastasis in Autochthonous Mouse Models of PDAC

Next, we tested the association between large 4C4 deletions and metastasis in an independent and autochthonous genetically engineered mouse model (GEMM) of PDAC. In agreement with a previous report³⁶, metastatic GEMM tumors initiated by mutant Kras^{G12D} alone or in combination with a TGFβ pathway alteration (Smad4 depletion in our model) spontaneously acquire 4C4 deletions during their natural course of tumor evolution (FIGS. 2J-2L). Analysis of deletion size revealed that primary tumor cells isolated from mice with metastases almost always harbored large 4C4 events (8/9 mice) whereas those without overt metastases had focal Cdkn2a b deletions or no 4C4 alteration (4/6 mice) (FIGS. 2K, 2L). The presence and extent of 4C4 deletion was similar between individual primary and metastatic pairs (n=7), confirming that 4C4 loss is an early event in this model (FIG. 7N). Nonetheless, in contrast to the PDEC system, primary tumors arising in this GEMM model displayed a moderately differentiated histology with stromal involvement (FIG. 2J), implying that the increased metastatic potential associated with large 4C4 deletions does not require an undifferentiated histopathology. These orthogonal data reinforce the notion that one or more genes unique to the ΔL deletion suppress metastasis.

### Example 8: 4C4 Deletion Genotype Dictates Type I IFN Signaling and Immune Infiltration

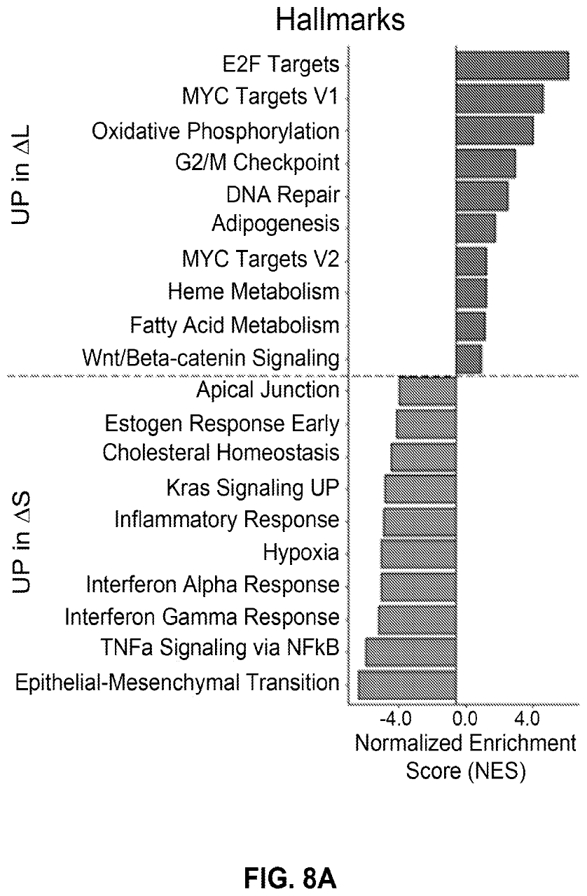

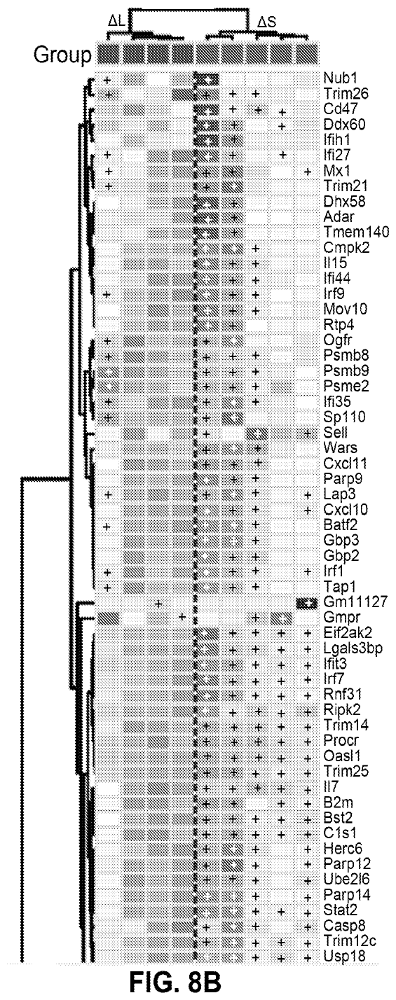

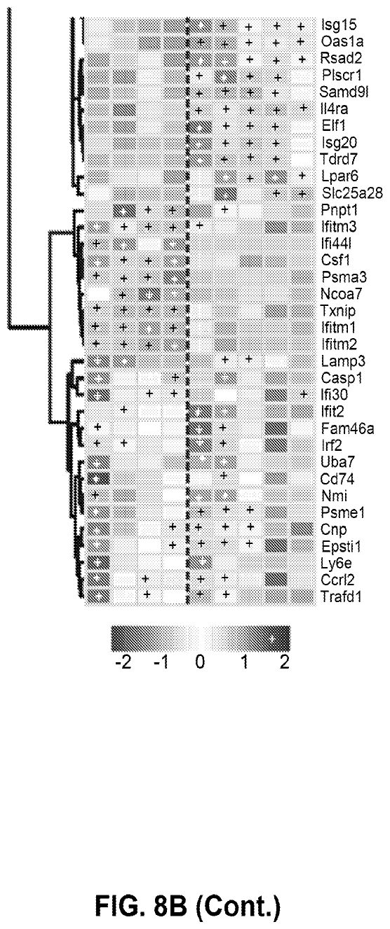

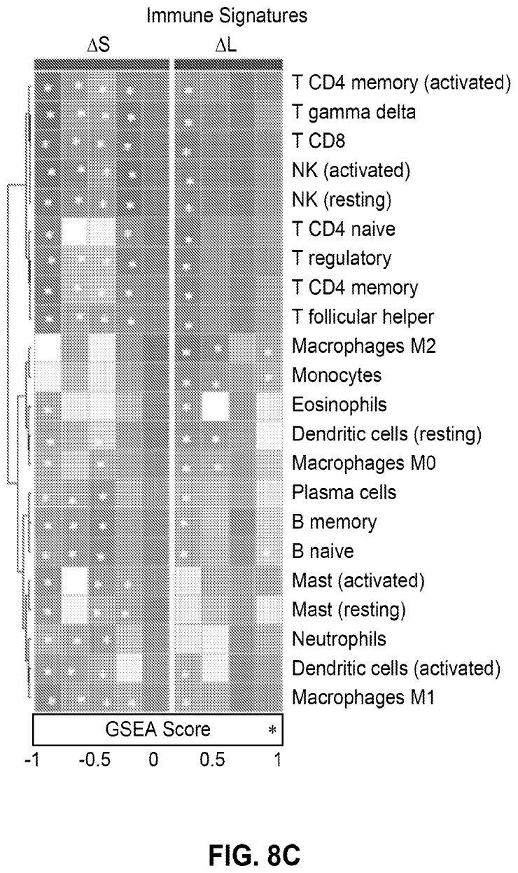

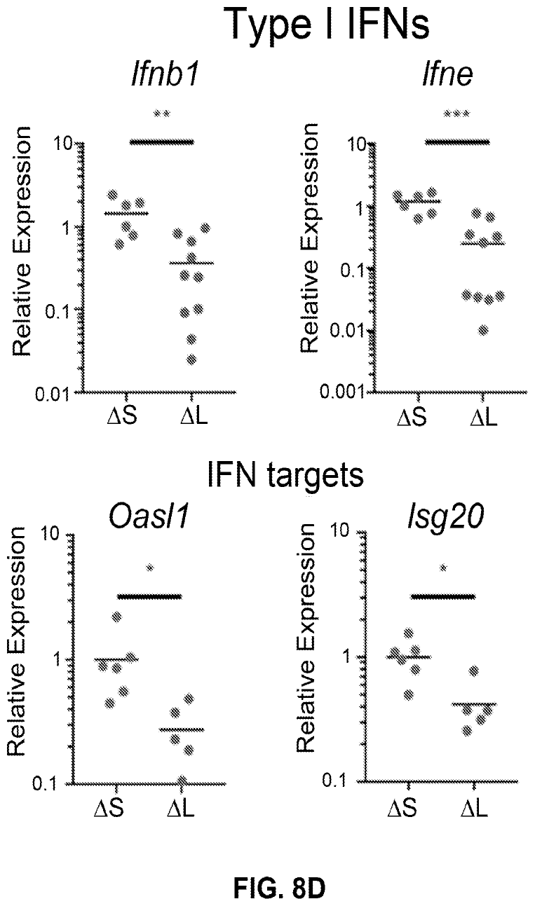

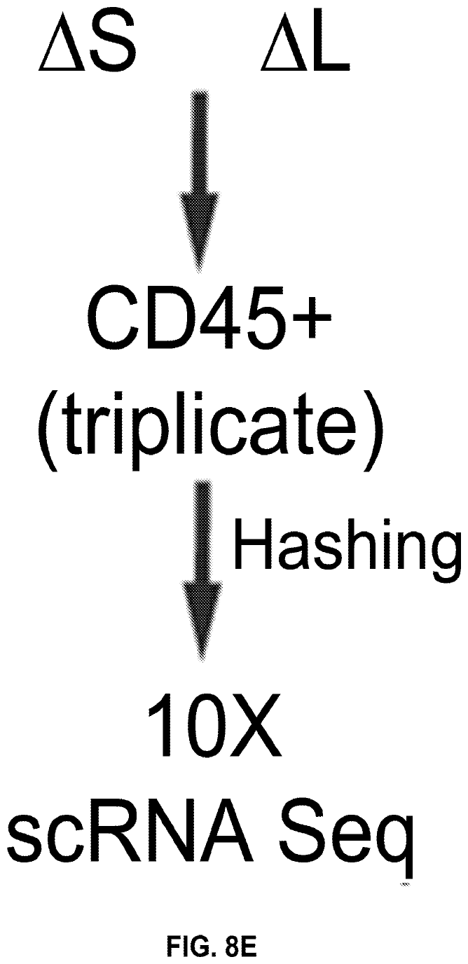

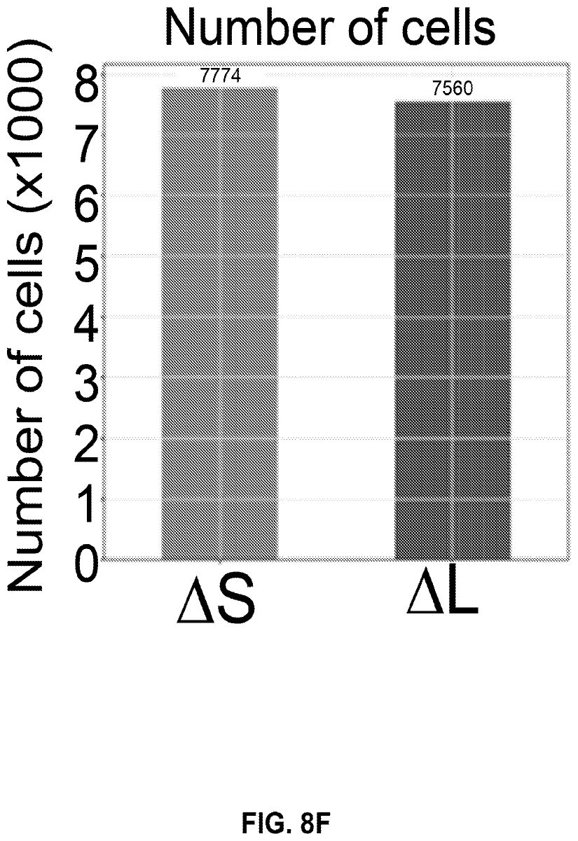

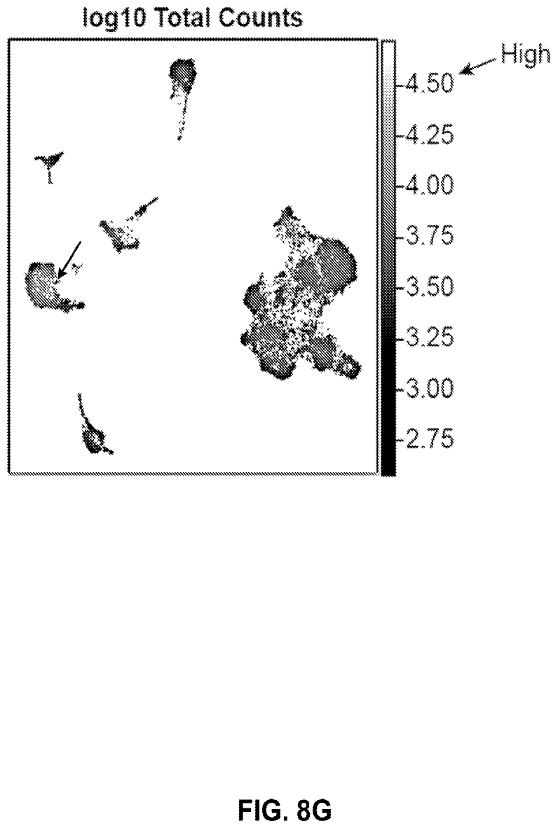

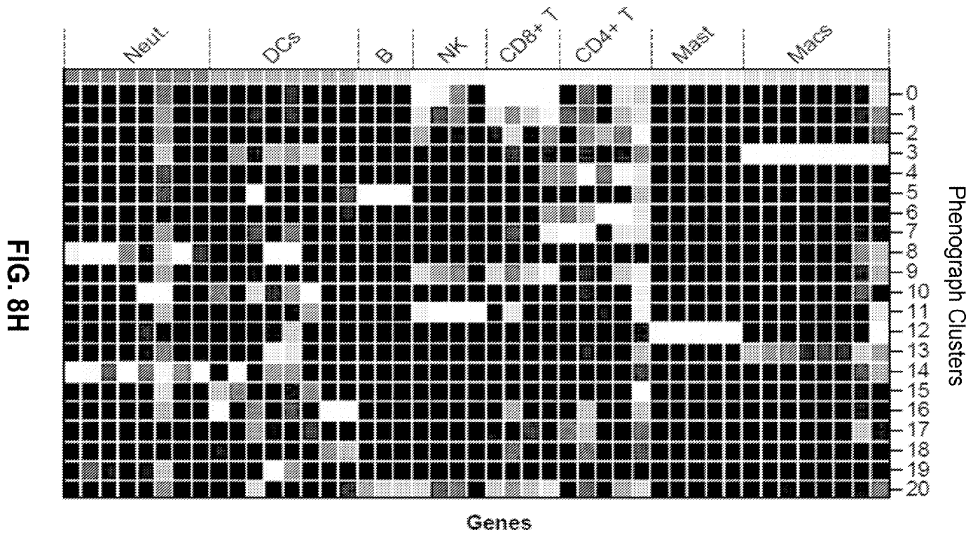

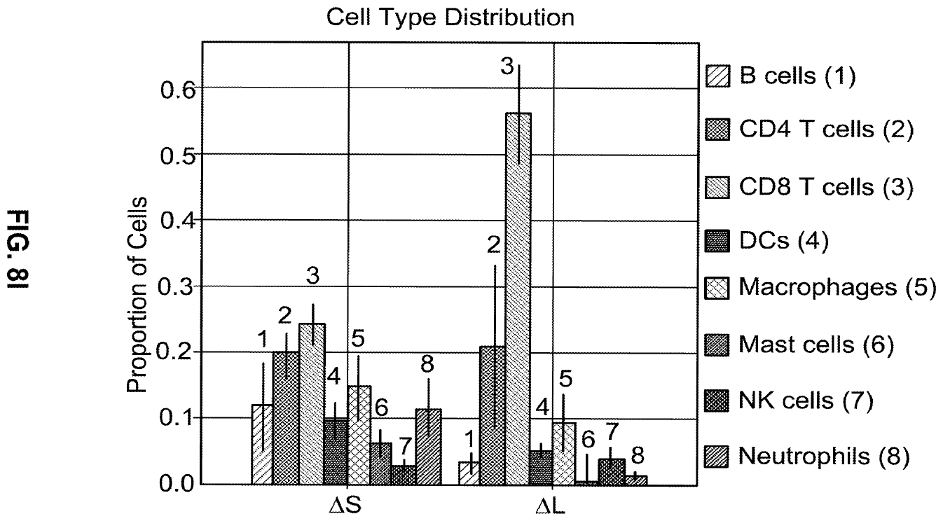

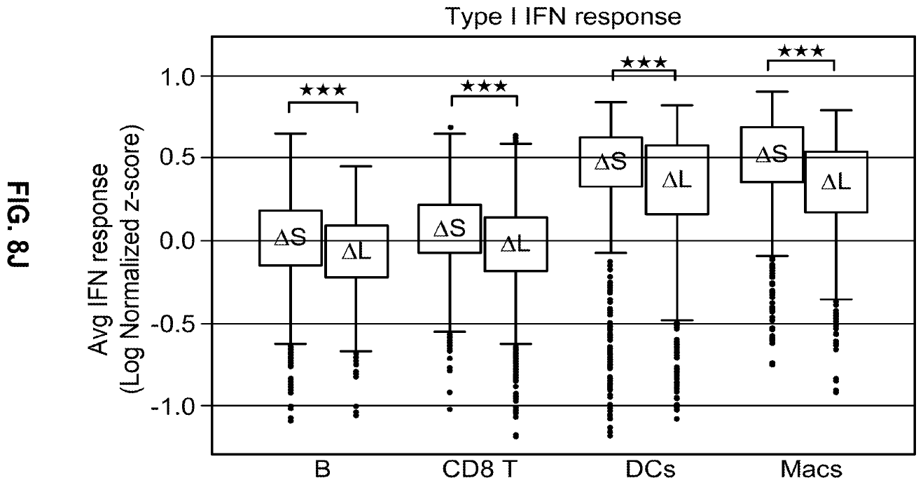

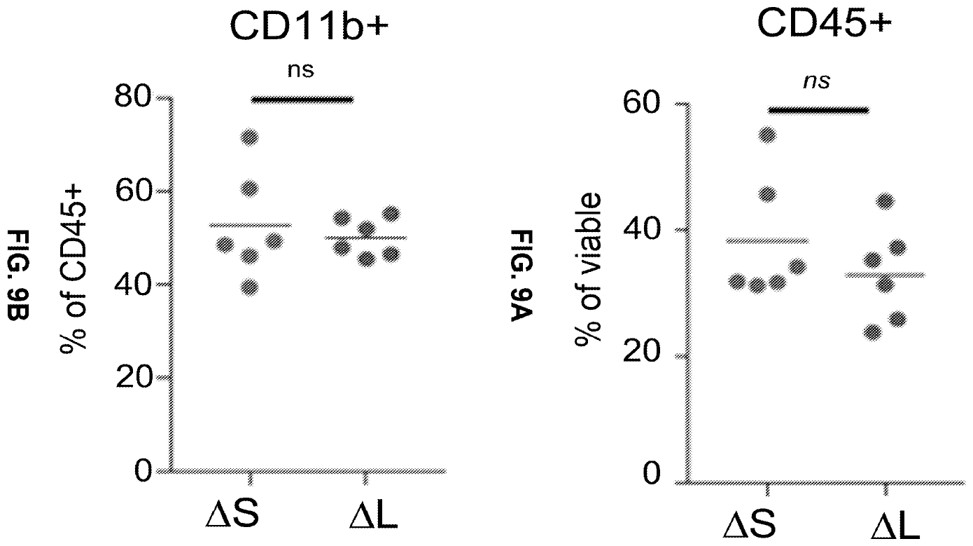

To help understand how distinct 4C4 deletion events influence tumor phenotypes, we performed RNA-seq on bulk ΔL and ΔS tumors and inferred differences in signaling pathways and immune cell composition using CIBERSORT⁴². When compared to ΔS tumors, ΔL tumors displayed a decrease in pathways linked to IFN signaling (FIGS. 8A-8B), as well as a broad depletion in immune signatures, including B and T cell populations (FIG. 8C). Further analyses using RT-qPCR confirmed that ΔL tumors have reduced levels of type I IFNs (Ifnb1 and Ifne) and IFN-responsive genes (Oasl1 and Isg20) (FIG. 8D). Adding granularity to these observations, single cell RNA sequencing (scRNA-seq) of tumor-infiltrating CD45+ cells isolated from ΔS and ΔL tumors identified changes in the abundance of multiple immune cell populations (FIGS. 8E-81). ΔL tumors had fewer B cells and myeloid populations, which was accompanied by an increase in CD8+ T cells-changes that were confirmed by flow cytometry (FIGS. 3A-3B, 9A-91).

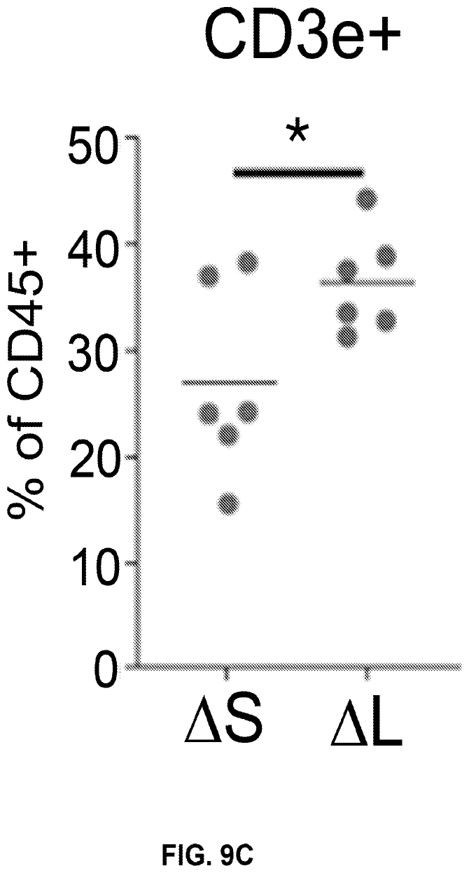

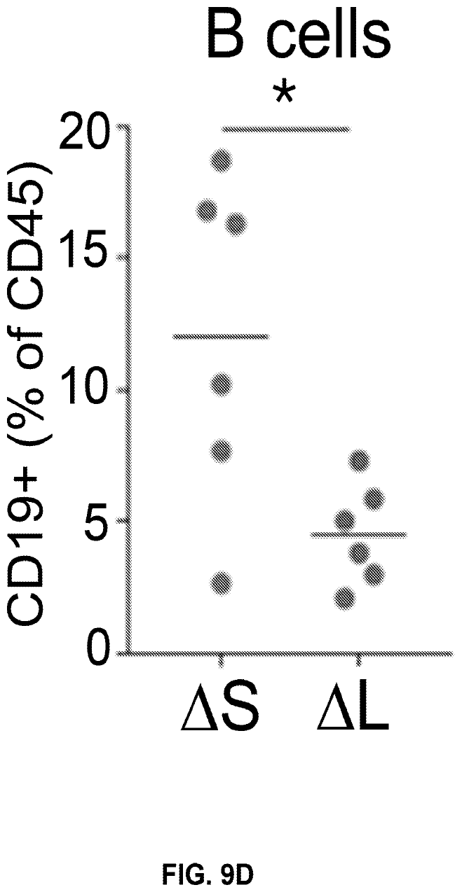

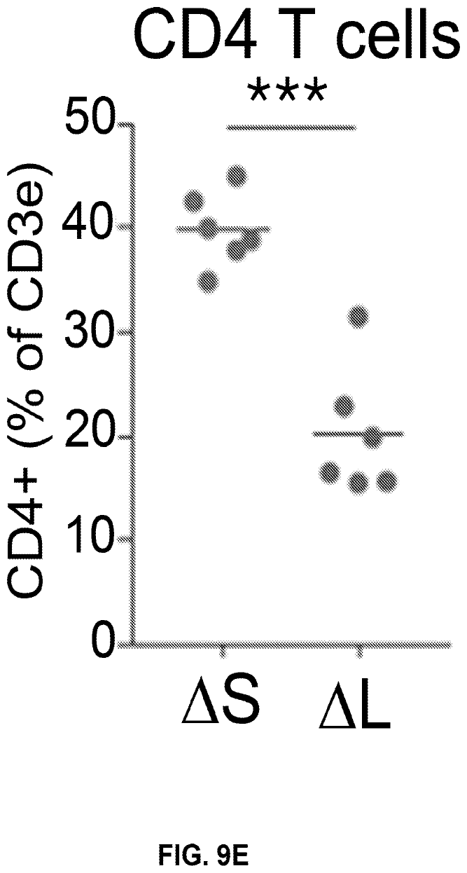

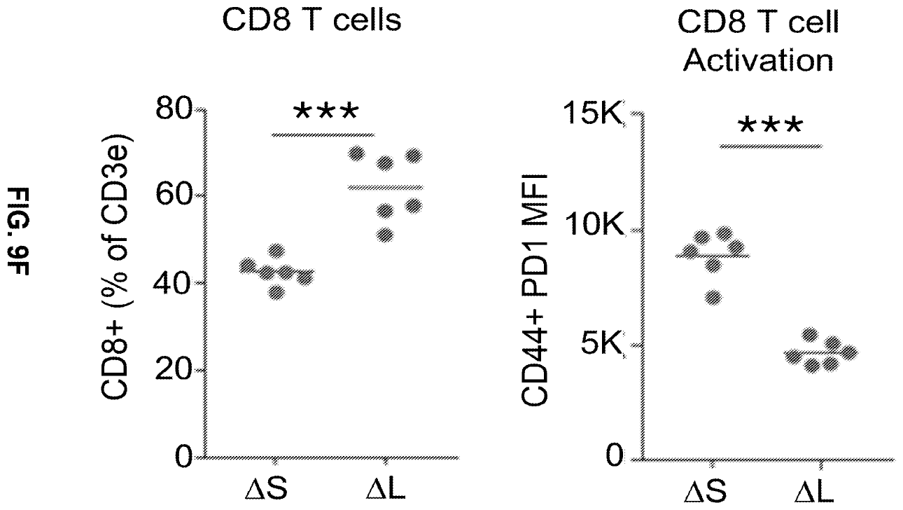

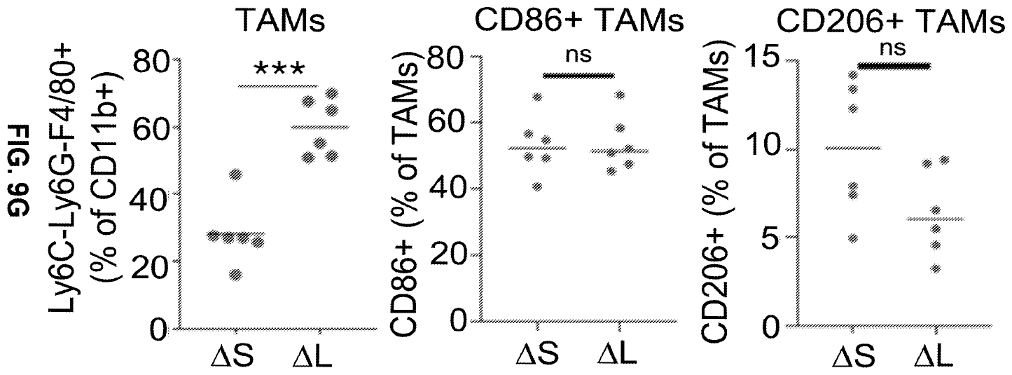

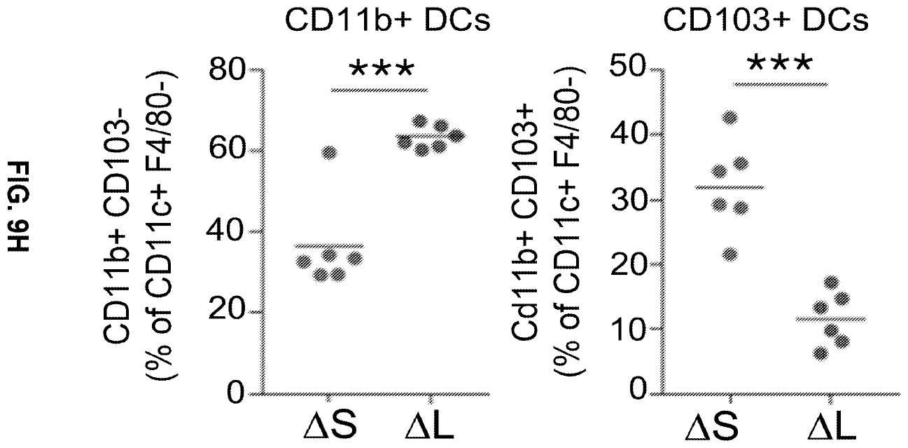

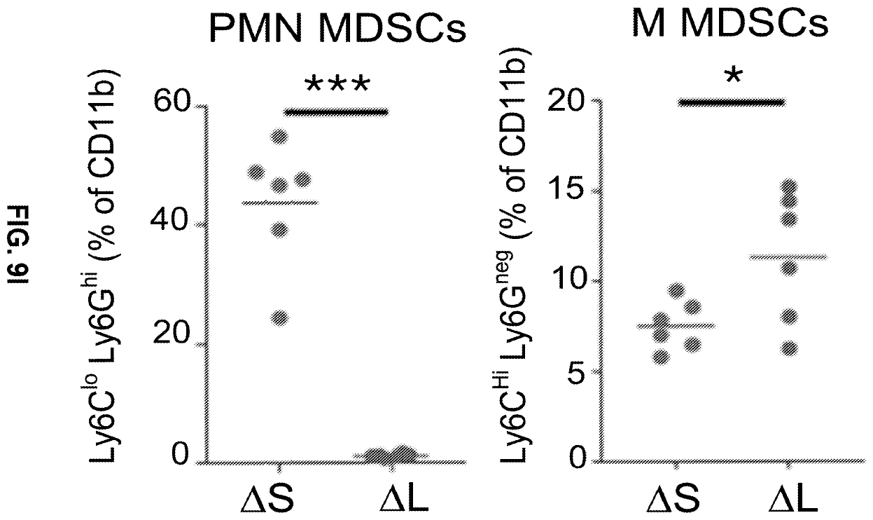

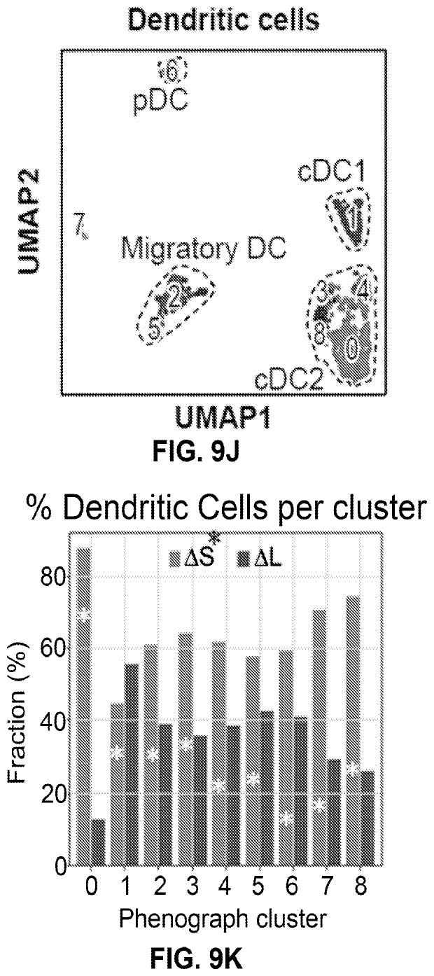

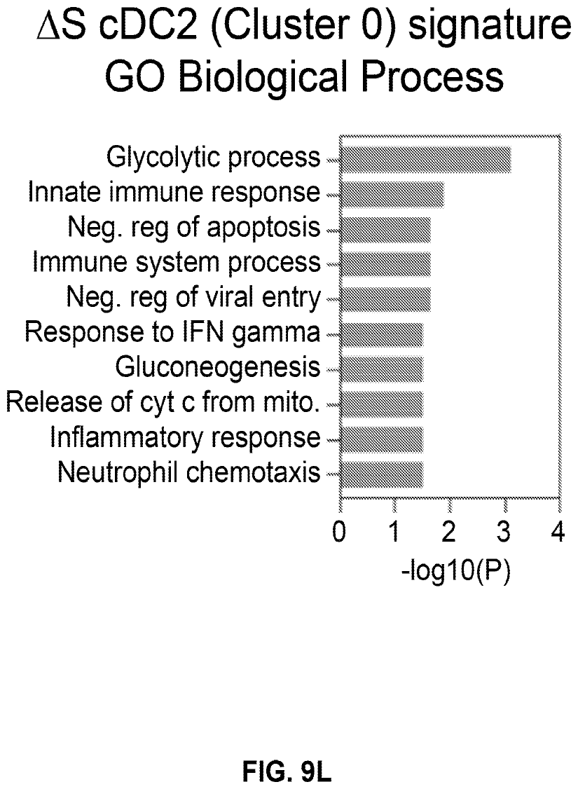

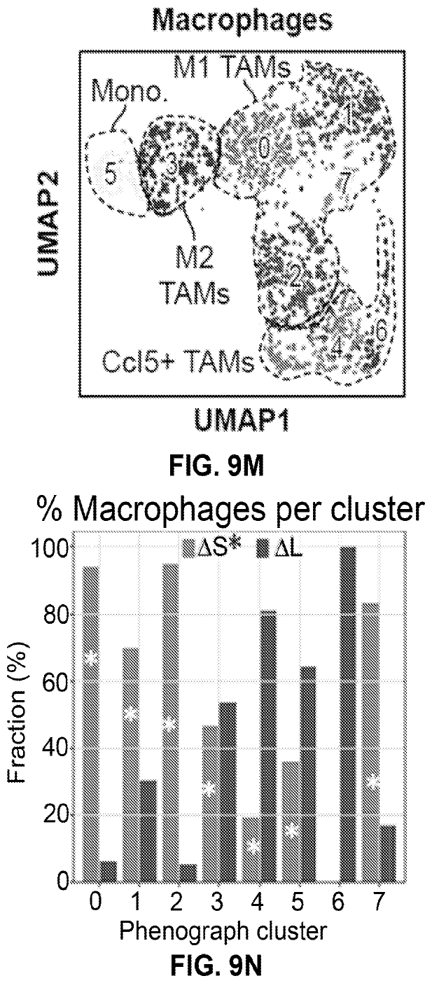

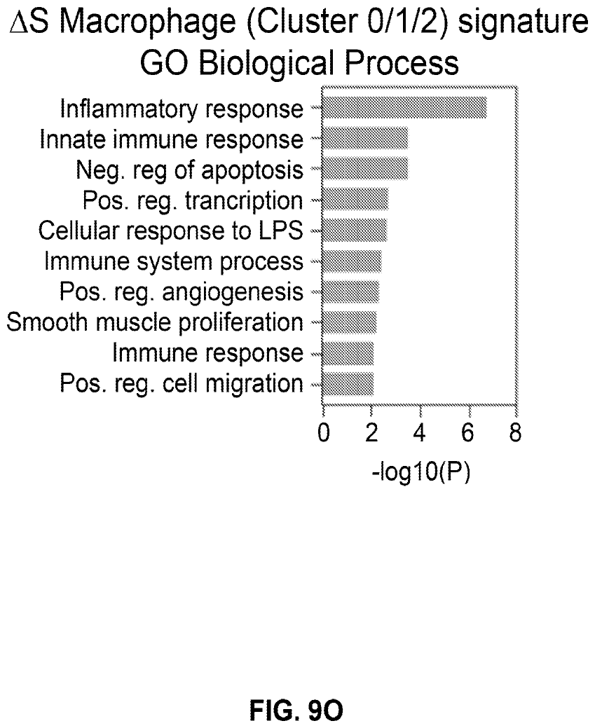

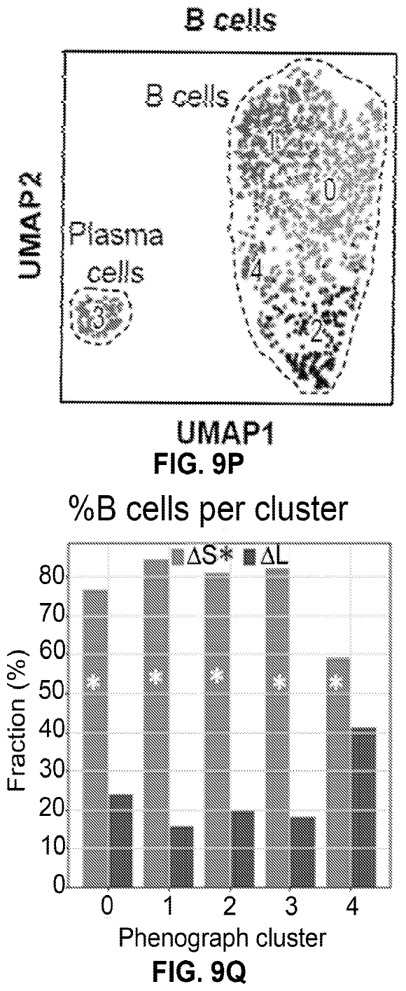

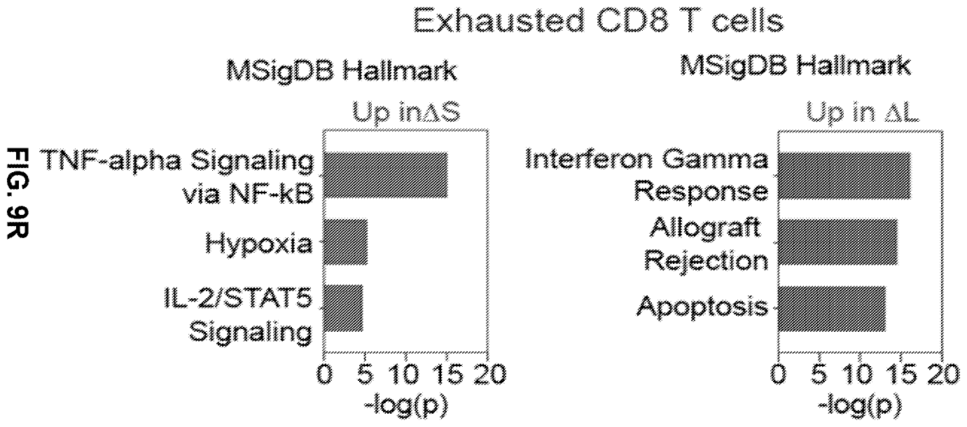

Beyond alterations in the composition of infiltrating CD45+ cells, the distinct 4C4 deletions led to changes in the transcriptional state of immune subsets. Analysis of an experimentally derived type I IFN response signature (see Methods, data not shown) showed that professional antigen-presenting cells (APCs; macrophages, dendritic cells and B cells) and CD8+ T cells exhibited reduced type I IFN signaling in the ΔL setting (FIGS. 3B-3C, FIG. 9J). Moreover, the specific effects of 4C4 deletions on APCs were immune cell type-dependent: a more pro-inflammatory state of cDC2 dendritic cells in ΔS tumors (FIGS. 9J-9L); a shift in macrophage transcriptional states toward higher M1-like cells in in ΔS tumors (FIGS. 9M-90); and an overall reduction across all B cell subtypes in ΔL tumors (FIGS. 9P-9Q).

Analysis of CD8+ T cells showed a range of activation states, with a dominant presence of activated/exhausted (Pdcd1+, Ctla4+, Haver2+, Lag3+), naïve (Pdcd1−, Tcf7+, Sell+), and cycling cells (Pdcd1+, MKi67+) (FIGS. 3D-3E). Intriguingly, the non-proliferating Pdcd1+ population of CD8+ T cells occupied distinct phenotypic space in AS and ΔL tumors. Further characterization using MILO⁴³ revealed that ΔS tumors accumulated exhausted CD8+ T cells marked by Tox and Bcl2 expression, whereas those present in ΔL tumors were transcriptionally distinct and displayed higher expression of Haver2 and Lag3 (FIGS. 3F-31, FIG. 9R). The high levels of IFN-engaged APCs and distinct CD8+ T cell states present in ΔS tumors implied ongoing immune surveillance that, based our phenotypic data, may suppress metastatic spread. In agreement, depletion of B and CD8+ cells, but not CD4+ cells, enhanced the metastatic potential of ΔS tumor cells to levels observed for ΔL tumors (FIGS. 3J-3K). Collectively, these data suggest that loss of tumor-intrinsic type I IFNs impairs the function of professional APCs and produces a unique state of CD8+ T cell dysfunction, leading to defects in anti-tumor immunity.

### Example 9: 9p21.3 Deletions Correlate with IFN Signaling and Immune Infiltration in Human PDAC

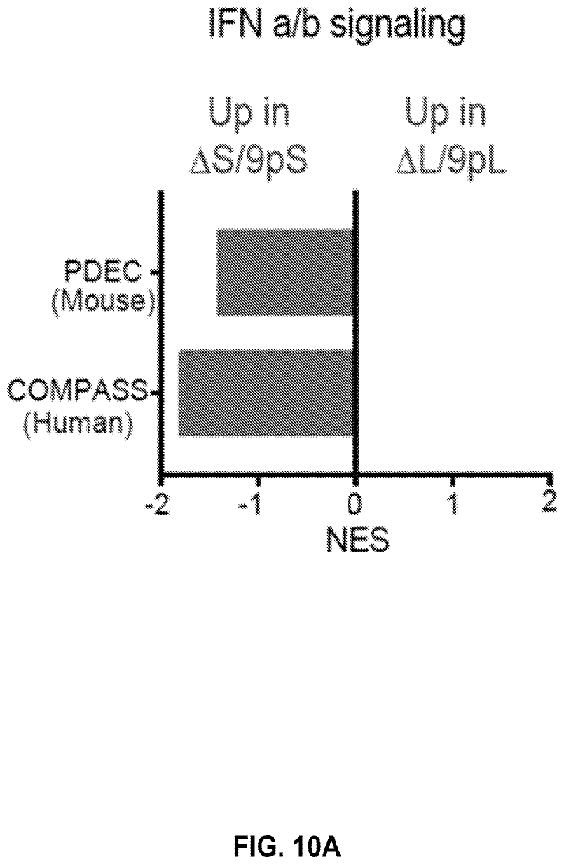

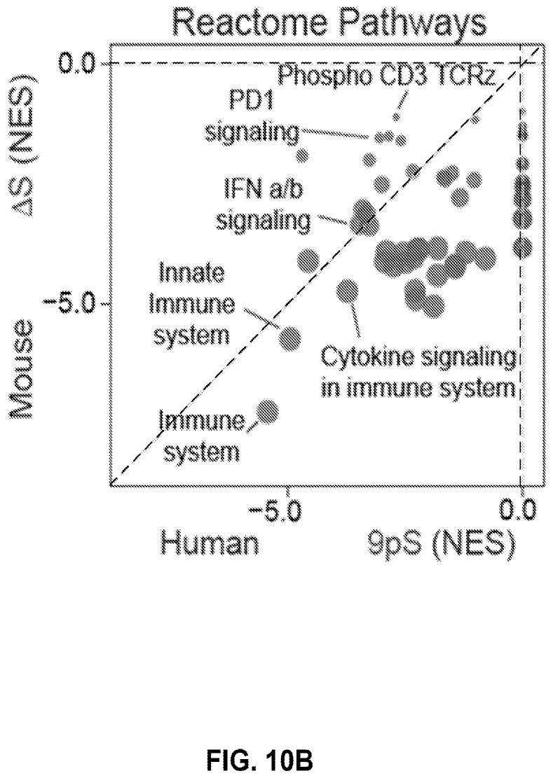

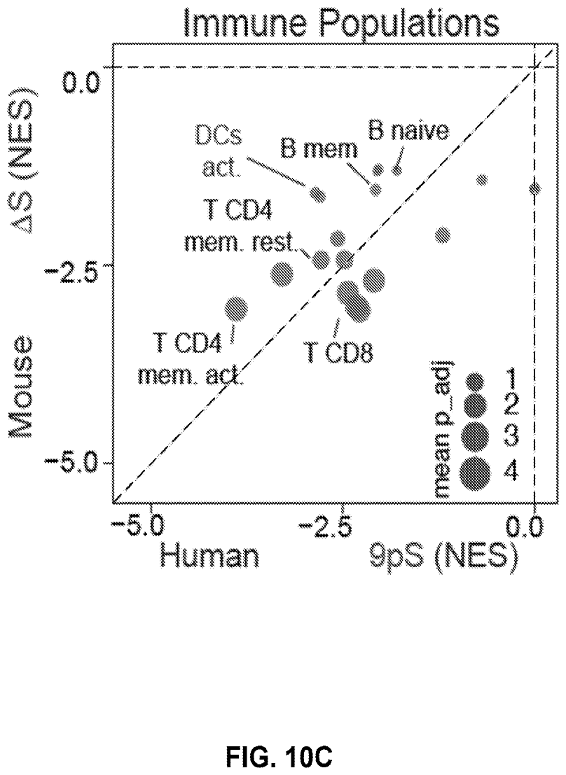

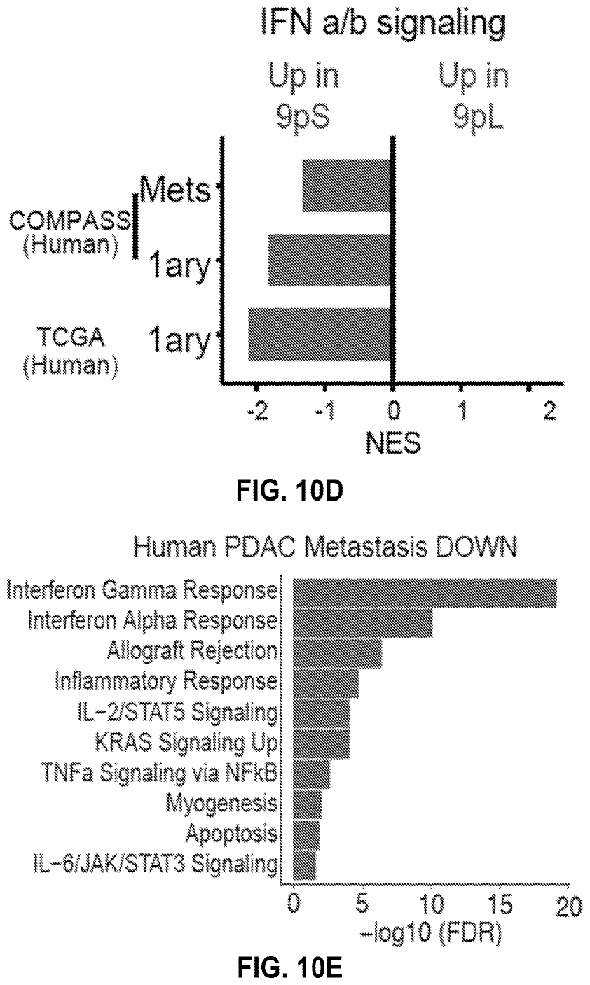

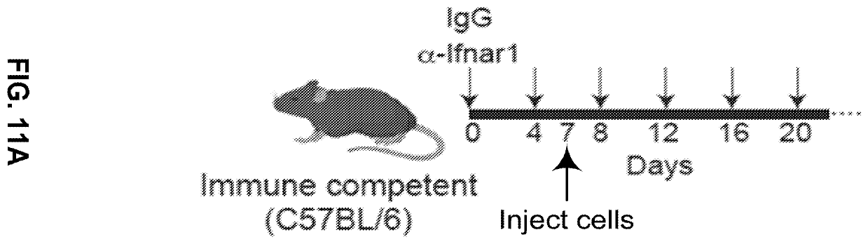

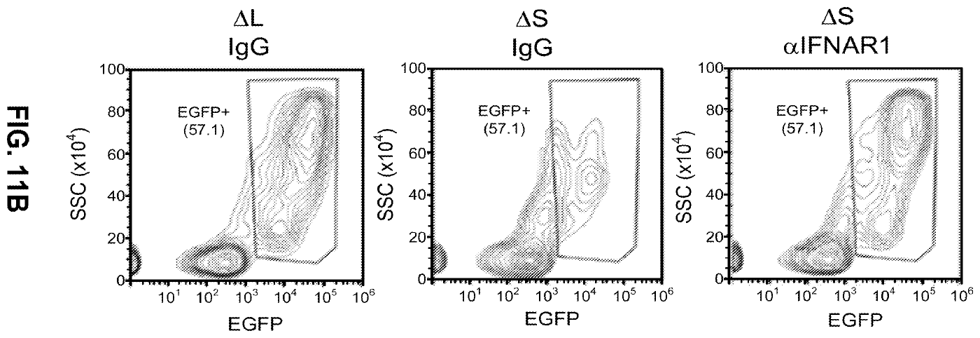

To test how 9p21.3 deletions that encompass the type I IFN cluster alter the tumor microenvironment in human PDAC, we analyzed sequencing data obtained from the COMPASS trial, which contains 218 primary and 180 metastatic PDAC samples isolated by laser capture microdissection^{44,45}. The availability of whole genome and RNA sequencing from each of these samples allows tumors to be categorized based on 9p deletion status and then analyzed for immune signatures linked to infiltrating stromal cells. Consistent with our findings in murine tumors, analysis of primary tumors showed that 9 pL deletions correlated with reduced type I IFN signaling compared to their 9pS counterparts (FIG. 10A).

The genotype-specific differences in gene ontology pathways and inferred immune cell composition correlated well across species (FIGS. 10B-10C). Notably, IFN cluster-proficient (AS/9pS) tumors were enriched in pathways associated with immune infiltration of both innate and adaptive categories (FIG. 10B) and showed a relative enrichment of most immune cell populations, particularly effector CD8+ T and B cell subsets (FIG. 10C). Nevertheless, the relative enrichment in type I IFN signatures present in primary 9pS tumors was reduced in 9pS metastases (FIG. 10D)⁴⁶, and analysis of RNA-seq data from a second cohort of matched primary and metastatic PDAC samples confirmed a reduction in type I IFN signaling in metastases irrespective of tumor genotype (FIG. 10E). When considered in the context of our functional studies, these data imply that downregulation of type I IFN signaling, by genetic or other means, promotes PDAC metastasis.

### Example 10: Disruption of IFNAR Signaling Phenocopies the Immune Evasive and Pro-Metastatic Properties of ΔL Cells

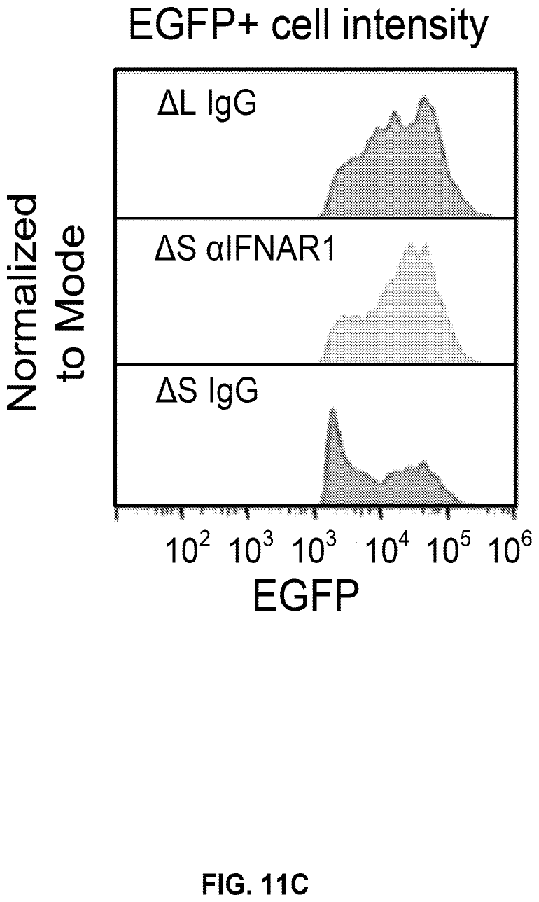

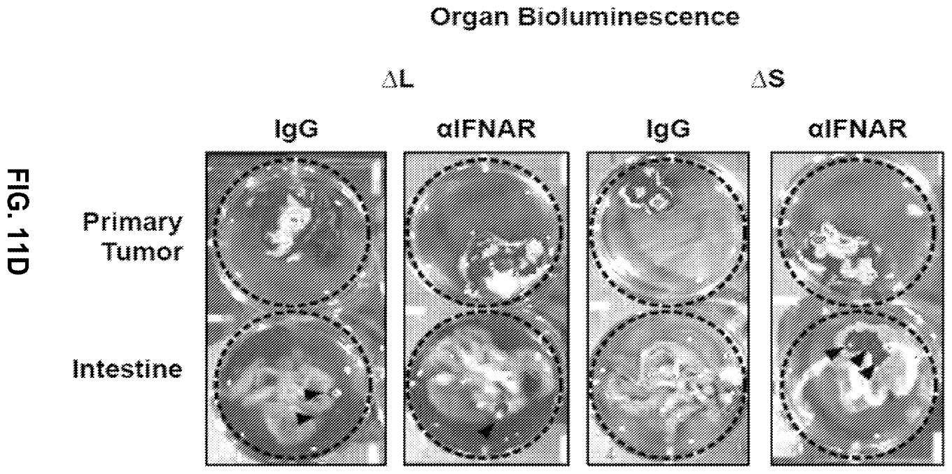

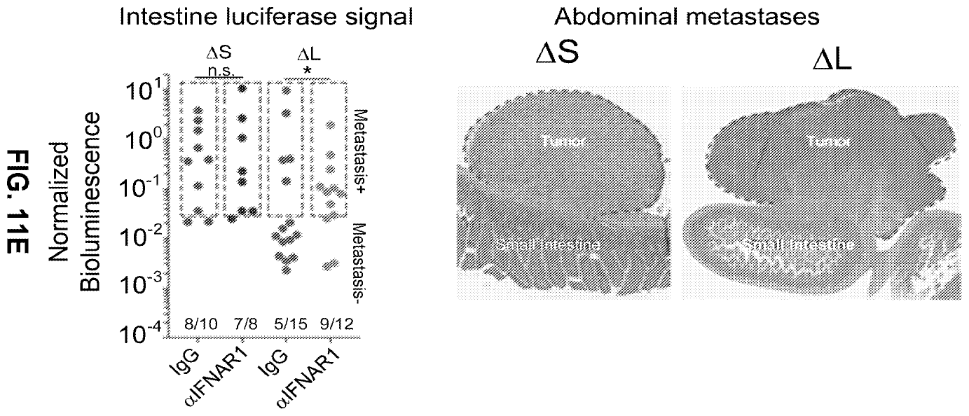

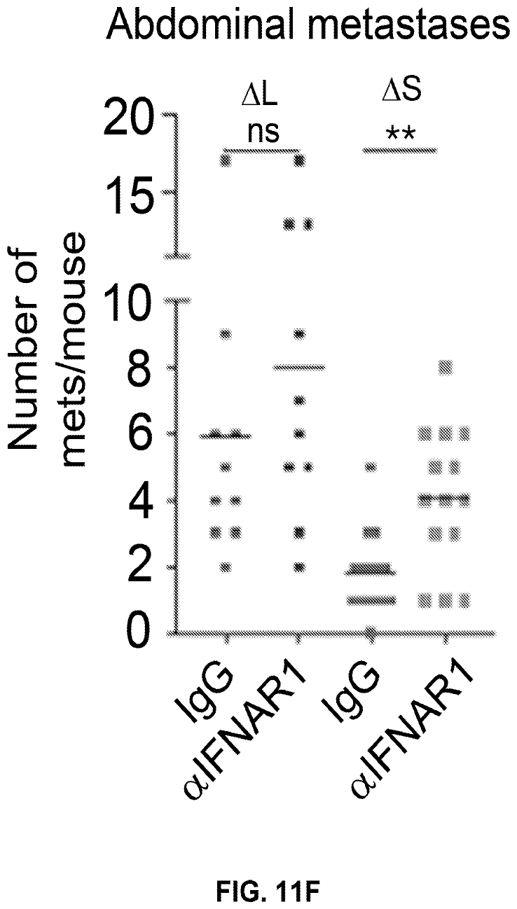

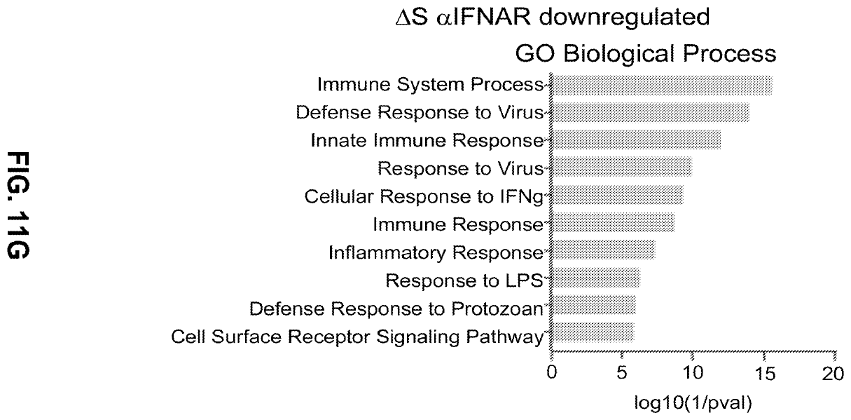

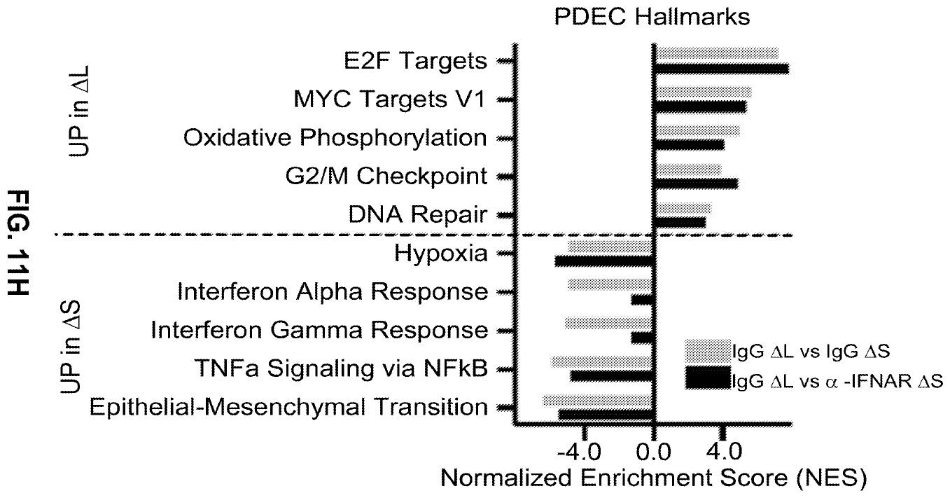

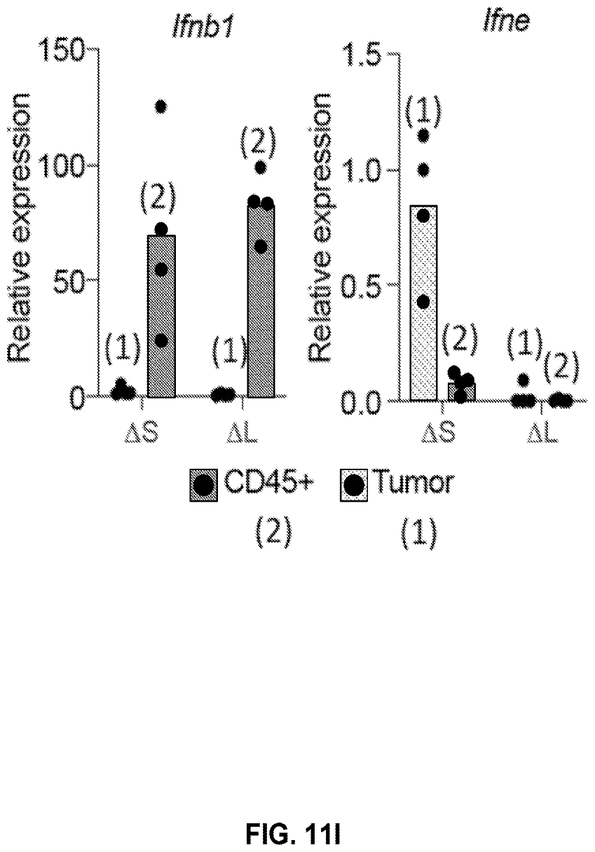

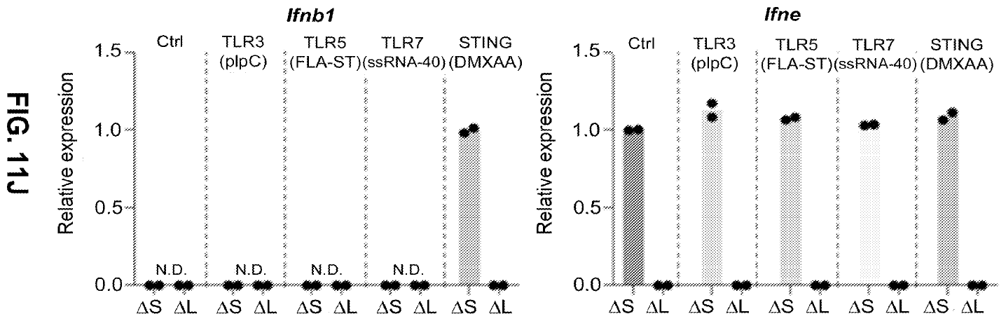

Besides type I IFNs, ΔL deletions include other genes, including Mtap, whose disruption can also influence tumor cell behavior⁴⁷. To specifically test whether type I IFN signaling is required for the immune evasive and pro-metastatic features of ΔL tumors, we used IFNARI blocking antibodies as an orthogonal approach to disrupting type I IFN signaling in the host. Immune competent mice were pre-treated with an IFNAR1-blocking antibody or an isotype control, followed by orthotopic transplantation of ΔS and ΔL cells analysis of the resulting tumors for immunoediting of the EGFP-Luciferase reporter and overall incidence of metastasis (FIG. 11A).

Consistent with our model, ΔS tumors arising in mice subjected to IFNAR1 blockade expressed higher levels of EGFP than isotype-treated controls (FIGS. 4A, 11B-11C) and showed a greater incidence of metastasis in secondary transplantation assays (FIGS. 4B; 11D-11F). Remarkably, these patterns were comparable to those arising in immune competent mice receiving ΔL cells and in immune deficient animals transplanted with ΔS cells (FIGS. 2C, 2F, 2I). In contrast, type I IFN blockade had no impact on the enhanced metastatic potential of ΔL cells (FIG. 4B). Transcriptional profiling of bulk tumors confirmed that IFNARI blockade phenocopied the reduction of type I IFN signaling observed in IFN-deficient tumors but had minimal impact on the transcriptome of AL tumors (FIG. 4C; FIGS. 11G-11H). These data imply that that one or more type I IFNs are required for the immune evasive and pro-metastatic phenotypes arising in tumors with homozygous ΔL deletions.

### Example 11: Ifne is a Tumor-Specific Mediator of Immune Surveillance and Metastasis

The functional redundancy between different type I IFNs remains poorly understood⁴⁸. For instance, Ifnb1 is highly expressed in immune cells and acts as a key downstream effector of the cGAS-STING pathway to engage innate and adaptive immunity, yet the individual contributions of most other IFNs to infection and cancer immunity are unclear^{30,49}. To dissect the functional contribution of tumor-derived IFNs to immunoediting and metastasis, we leveraged the power of MACHETE to engineer a refined deletion series that encompass a gradually increasing number of IFN genes (FIG. 4D). The resulting cell populations were orthotopically injected as pools into immunocompetent recipient mice (FIG. 4E) and expression of EGFP-Luc reporter was used as an indicator of immune evasion in the resulting tumors.

Consistent with different deletion events affording different degrees of immune evasion, the tumors showed heterogenous expression of EGFP (FIG. 4F). Isolation of cells with distinct levels of EGFP showed prevalence in deletions affecting the IFN cluster in the EGFP-retaining population (FIG. 4G), with a significant enrichment of cells harboring deletions of Ifne across multiple independent tumors (FIG. 4H). A similar increase in the deletion of Ifne was observed when comparing metastases to primary tumors, further highlighting the potential relevance of Ifne to tumor dissemination (FIG. 4I).

A detailed analysis of type I IFN gene expression in epithelial and CD45+immune cells present in ΔS tumors reinforced the above observations. As previously reported, Ifnb1 could be induced by a cGAS-STING agonist yet was more highly expressed in immune cells than tumor cells; by contrast, other IFNs, particularly Ifne, were not induced by these stimuli and showed preferential expression in tumor cells (FIG. 4J, FIGS. 11I-11J). Collectively these data imply that disruption of Ifne is necessary for the effects of type I IFN loss on immune evasion and metastasis.

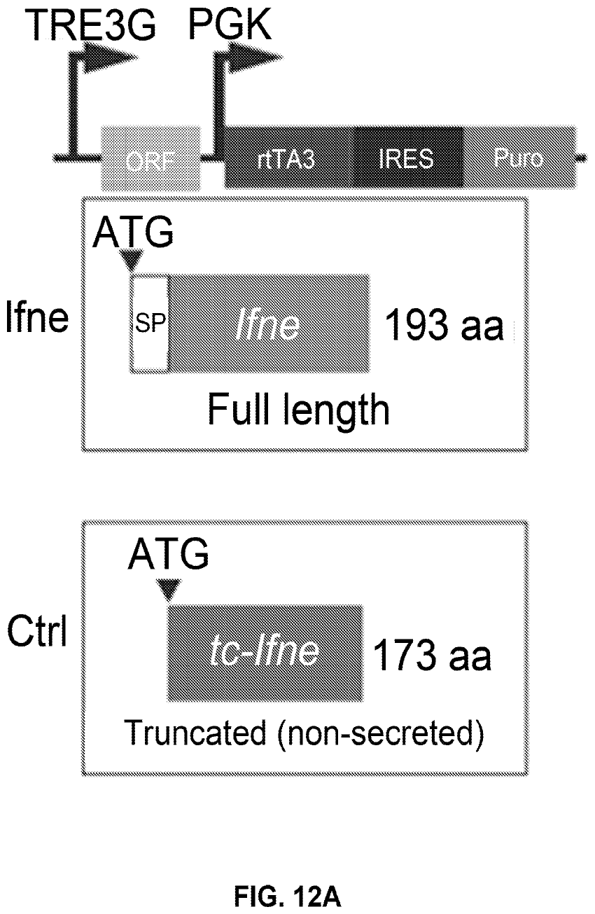

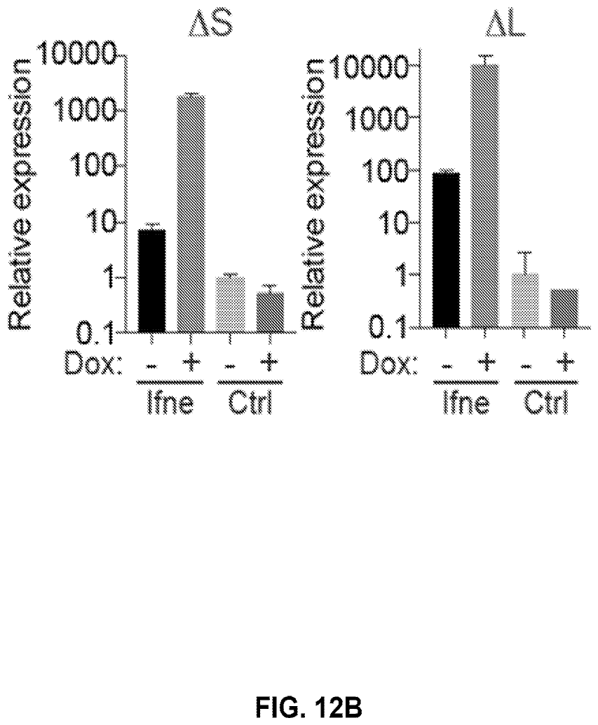

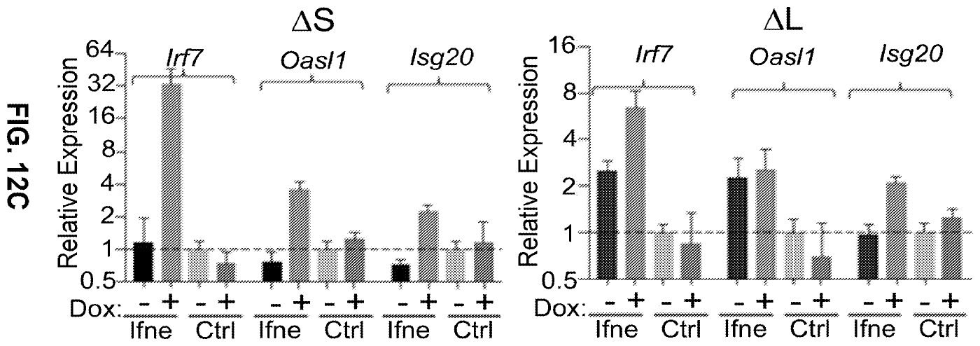

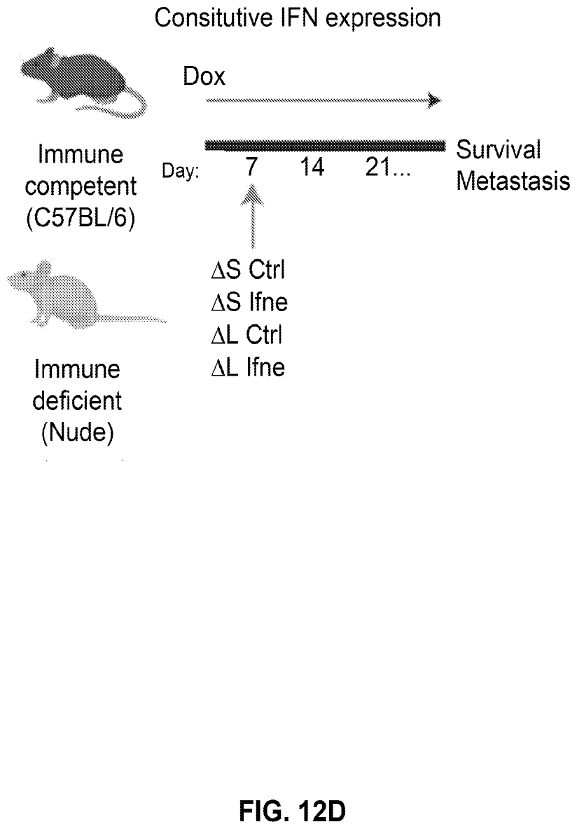

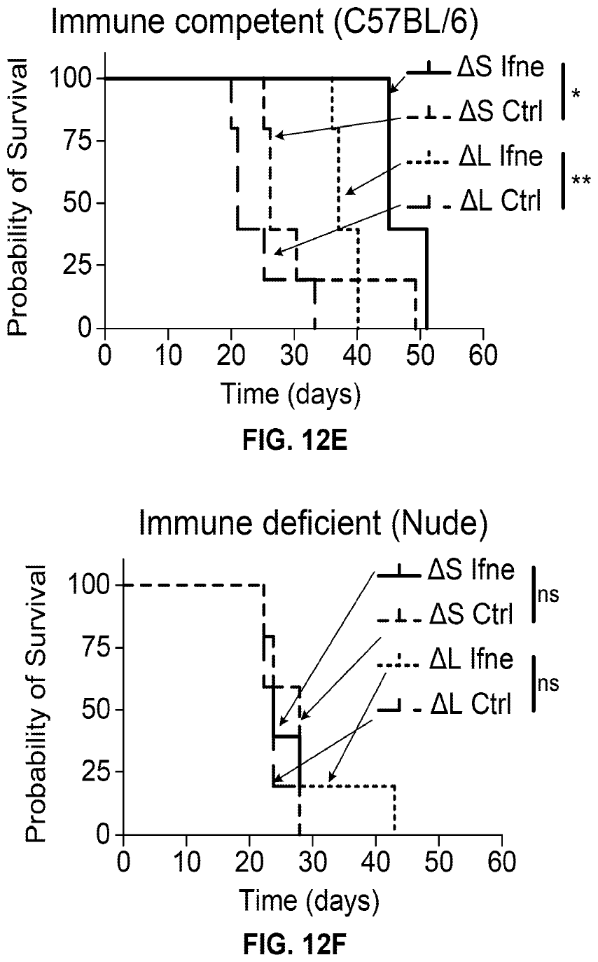

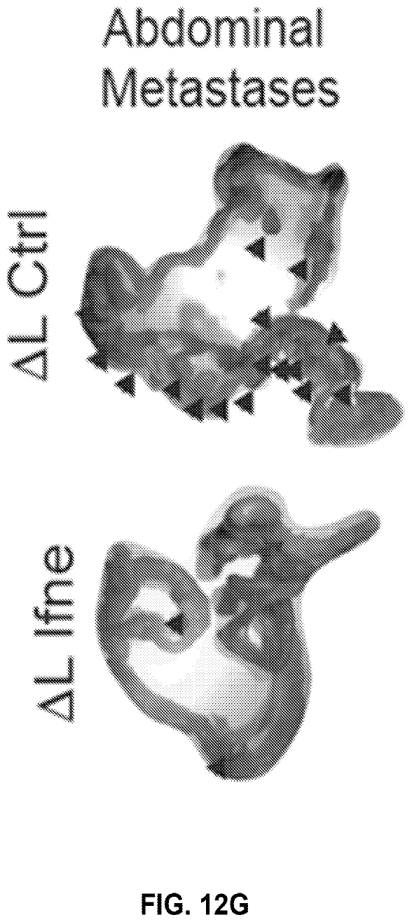

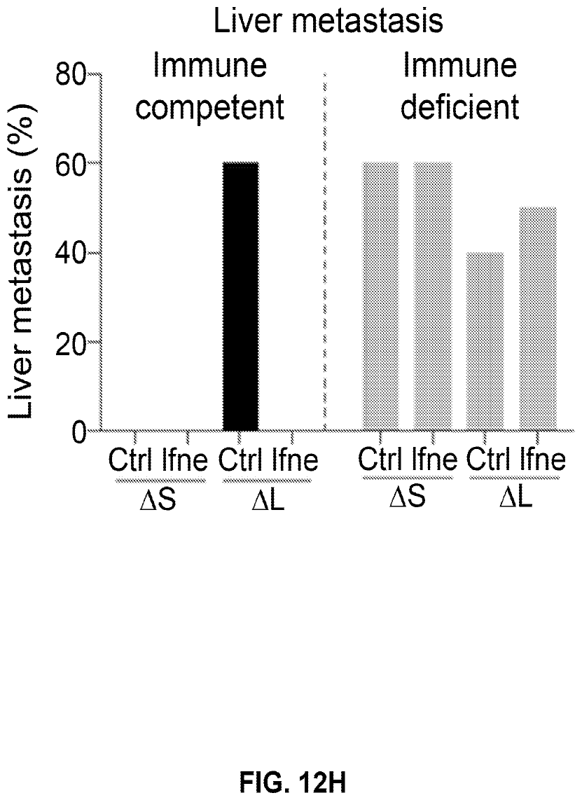

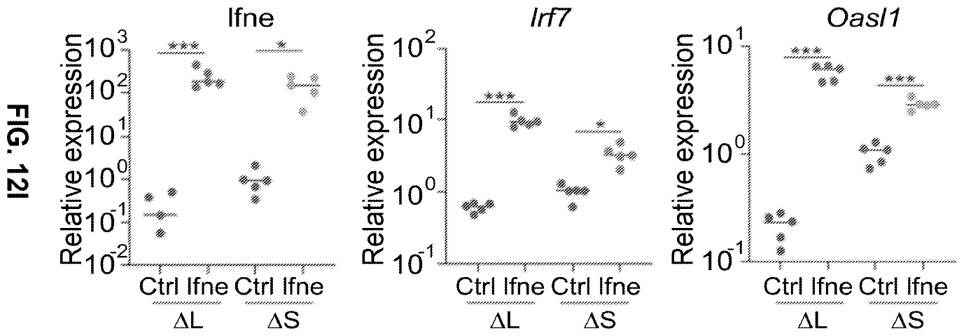

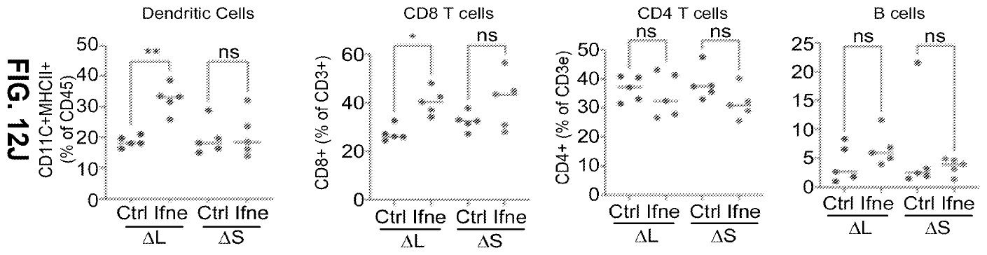

To determine whether Ifne was sufficient to suppress immune evasion and metastasis, we introduced a doxycycline-inducible construct to drive either full-length Ifne or a truncated Ifne control in ΔS and ΔL cells (FIGS. 12A-12C). Both sustained and acute induction of full-length Ifne suppressed overt metastasis of ΔL tumors, which was dependent on adaptive immunity (FIG. 4K, FIGS. 12D-12H). Despite the expected overexpression of Ifne and downstream type I IFN target genes (FIG. 12I), ΔS and AL tumors showed differential response to acute Ifne: ΔS tumors had no effect on primary tumor growth while ΔL tumors had a reduction in tumor size and metastasis (FIG. 4K). Consistent with loss of function phenotypes, tumors with enforced Ifne expression displayed elevated levels of professional antigen-presenting populations and an increase in activated CD8+ T cells (FIGS. 4L-4M, 12J). Taken together, these data demonstrate that somatic deletion of type I IFNs impairs immunoediting and metastasis via the adaptive immune system and reveal a previously unanticipated role of Ifne in suppressing these phenotypes.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

### REFERENCES

1 Stratton, M. R., Campbell, P. J. & Futreal, P. A. The cancer genome. Nature 458, 719-724, doi: 10.1038/nature07943 (2009).
2 Vasudevan, A. et al. Aneuploidy as a promoter and suppressor of malignant growth. Nat Rev Cancer, doi: 10.1038/s41568-020-00321-1 (2021).
3 Beroukhim, R. et al. The landscape of somatic copy-number alteration across human cancers. Nature 463, 899-905, doi: 10.1038/nature08822 (2010).
4 Zack, T. I. et al. Pan-cancer patterns of somatic copy number alteration. Nat Genet 45, 1134-1140, doi: 10.1038/ng.2760 (2013).
5 Sherr, C. J. The INK4a/ARF network in tumour suppression. Nat Rev Mol Cell Biol 2, 731-737, doi: 10.1038/35096061 (2001).
6 Mitelman, F. Recurrent chromosome aberrations in cancer. Mutation research 462, 247-253, doi: 10.1016/s1383-5742 (00) 00006-5 (2000).
7 Smith, J. C. & Sheltzer, J. M. Systematic identification of mutations and copy number alterations associated with cancer patient prognosis. Elife 7, doi: 10.7554/eLife.39217 (2018).
8 Li, Y. et al. Patterns of somatic structural variation in human cancer genomes. Nature 578, 112-121, doi: 10.1038/s41586-019-1913-9 (2020).
9 Kumar, S. et al. Passenger Mutations in More Than 2,500 Cancer Genomes: Overall Molecular Functional Impact and Consequences. Cell 180, 915-927.e916, doi: 10.1016/j.cell.2020.01.032 (2020).
10 Muller, F. L., Aquilanti, E. A. & DePinho, R. A. Collateral Lethality: A new therapeutic strategy in oncology. Trends Cancer 1, 161-173, doi: 10.1016/j.trecan.2015.10.002 (2015).
11 Xue, W. et al. A cluster of cooperating tumor-suppressor gene candidates in chromosomal deletions. Proc Natl Acad Sci USA 109, 8212-8217, doi: 10.1073/pnas. 1206062109 (2012).
12 Cai, Y. et al. Loss of Chromosome 8p Governs Tumor Progression and Drug Response by Altering Lipid Metabolism. Cancer Cell 29, 751-766, doi: 10.1016/j.ccell.2016.04.003 (2016).
13 Liu, Y. et al. Deletions linked to TP53 loss drive cancer through p53-independent mechanisms. Nature 531, 471-475, doi: 10.1038/nature17157 (2016).
14 Scuoppo, C. et al. A tumour suppressor network relying on the polyamine-hypusine axis. Nature 487, 244-248, doi: 10.1038/nature11126 (2012).
15 Bonney, M. E., Moriya, H. & Amon, A. Aneuploid proliferation defects in yeast are not driven by copy number changes of a few dosage-sensitive genes. Genes Dev 29, 898-903, doi: 10.1101/gad.261743.115 (2015).
16 Solimini, N. L. et al. Recurrent hemizygous deletions in cancers may optimize proliferative potential. Science 337, 104-109, doi: 10.1126/science. 1219580 (2012).
17 Davoli, T. et al. Cumulative haploinsufficiency and triplosensitivity drive aneuploidy patterns and shape the cancer genome. Cell 155, 948-962, doi: 10.1016/j.cell.2013.10.011 (2013).
18 Taylor, A. M. et al. Genomic and Functional Approaches to Understanding Cancer Aneuploidy. Cancer Cell 33, 676-689 e673, doi: 10.1016/j.ccell.2018.03.007 (2018).
19 Mishra, A. et al. Generation of focal mutations and large genomic deletions in the pancreas using inducible in vivo genome editing. Carcinogenesis 41, 334-344, doi: 10.1093/carcin/bgz108 (2020).
20 He, Z. et al. Highly efficient targeted chromosome deletions using CRISPR/Cas9. Biotechnology and bioengineering 112, 1060-1064, doi: 10.1002/bit.25490 (2015).
21 Boroviak, K., Doe, B., Banerjee, R., Yang, F. & Bradley, A. Chromosome engineering in zygotes with CRISPR/Cas9. Genesis 54, 78-85, doi: 10.1002/dvg.22915 (2016).
22 Serrano, M., Lin, A. W., McCurrach, M. E., Beach, D. & Lowe, S. W. Oncogenic ras provokes premature cell senescence associated with accumulation of p53 and p16INK4a. Cell 88, 593-602, doi: 10.1016/s0092-8674 (00) 81902-9 (1997).
23 Kamijo, T. et al. Tumor suppression at the mouse INK4a locus mediated by the alternative reading frame product p19ARF. Cell 91, 649-659, doi: 10.1016/s0092-8674 (00) 80452-3 (1997).
24 Hannon, G. J. & Beach, D. p15INK4B is a potential effector of TGF-beta-induced cell cycle arrest. Nature 371, 257-261, doi: 10.1038/371257a0 (1994).
25 Balli, D., Rech, A. J., Stanger, B. Z. & Vonderheide, R. H. Immune Cytolytic Activity Stratifies Molecular Subsets of Human Pancreatic Cancer. Clin Cancer Res 23, 3129-3138, doi: 10.1158/1078-0432.CCR-16-2128 (2017).
26 Siemers, N. O. et al. Genome-wide association analysis identifies genetic correlates of immune infiltrates in solid tumors. PLOS One 12, e0179726, doi: 10.1371/journal.pone.0179726 (2017).
27 Gao, J. et al. Loss of IFN-gamma Pathway Genes in Tumor Cells as a Mechanism of Resistance to Anti-CTLA-4 Therapy. Cell 167, 397-404 e399, doi: 10.1016/j.cell.2016.08.069 (2016).
28 Braun, D. A. et al. Interplay of somatic alterations and immune infiltration modulates response to PD-1 blockade in advanced clear cell renal cell carcinoma. Nat Med 26, 909-918, doi: 10.1038/s41591-020-0839-y (2020).
29 Jeck, W. R., Siebold, A. P. & Sharpless, N. E. Review: a meta-analysis of GWΔS and age-associated diseases. Aging cell 11, 727-731, doi: 10.1111/j. 1474-9726.2012.00871.x (2012).
30 Parker, B. S., Rautela, J. & Hertzog, P. J. Antitumour actions of interferons: implications for cancer therapy. Nat Rev Cancer 16, 131-144, doi: 10.1038/nrc.2016.14 (2016).
31 Hoadley, K. A. et al. Cell-of-Origin Patterns Dominate the Molecular Classification of 10,000 Tumors from 33 Types of Cancer. Cell 173, 291-304.e296, doi: 10.1016/j.cell.2018.03.022 (2018).
32 Hruban, R. H., Goggins, M., Parsons, J. & Kern, S. E. Progression model for pancreatic cancer. Clin Cancer Res 6, 2969-2972 (2000).
33 Makohon-Moore, A. P. et al. Precancerous neoplastic cells can move through the pancreatic ductal system. Nature 561, 201-205, doi: 10.1038/s41586-018-0481-8 (2018).
34 Sasaki, S. et al. Molecular processes of chromosome 9p21 deletions in human cancers. Oncogene 22, 3792-3798, doi: 10.1038/sj.onc. 1206589 (2003).
35 Harada, T. et al. Genome-wide DNA copy number analysis in pancreatic cancer using high-density single nucleotide polymorphism arrays. Oncogene 27, 1951-1960, doi: 10.1038/sj.onc. 1210832 (2008).
36 Mueller, S. et al. Evolutionary routes and KRΔS dosage define pancreatic cancer phenotypes. Nature 554, 62-68, doi: 10.1038/nature25459 (2018).
37 Pylayeva-Gupta, Y., Lee, K. E., Hajdu, C. H., Miller, G. & Bar-Sagi, D. Oncogenic Kras-induced GM-CSF production promotes the development of pancreatic neoplasia. Cancer Cell 21, 836-847, doi: 10.1016/j.ccr.2012.04.024 (2012).
38 Lee, K. E. & Bar-Sagi, D. Oncogenic KRas suppresses inflammation-associated senescence of pancreatic ductal cells. Cancer Cell 18, 448-458, doi: 10.1016/j.ccr.2010.10.020 (2010).
39 Pylayeva-Gupta, Y. et al. IL35-Producing B Cells Promote the Development of Pancreatic Neoplasia. Cancer Discov 6, 247-255, doi: 10.1158/2159-8290.Cd-15-0843 (2016).
40 Bardeesy, N. et al. Both p16 (Ink4a) and the p19 (Arf)-p53 pathway constrain progression of pancreatic adenocarcinoma in the mouse. Proc Natl Acad Sci USA 103, 5947-5952, doi: 10.1073/pnas.0601273103 (2006).
41 DuPage, M., Mazumdar, C., Schmidt, L. M., Cheung, A. F. & Jacks, T. Expression of tumour-specific antigens underlies cancer immunoediting. Nature 482, 405-409, doi: 10.1038/nature10803 (2012).
42 Newman, A. M. et al. Robust enumeration of cell subsets from tissue expression profiles. Nat Methods 12, 453-457, doi: 10.1038/nmeth.3337 (2015).
43 Dann, E., Henderson, N. C., Teichmann, S. A., Morgan, M. D. & Marioni, J. C. Differential abundance testing on single-cell data using k-nearest neighbor graphs. Nat Biotechnol 40, 245-253, doi: 10.1038/s41587-021-01033-z (2022).
44 Aung, K. L. et al. Genomics-Driven Precision Medicine for Advanced Pancreatic Cancer: Early Results from the COMPASS Trial. Clin Cancer Res 24, 1344-1354, doi: 10.1158/1078-0432.Ccr-17-2994 (2018).
45 Chan-Seng-Yue, M. et al. Transcription phenotypes of pancreatic cancer are driven by genomic events during tumor evolution. Nat Genet 52, 231-240, doi: 10.1038/s41588-019-0566-9 (2020).
46 Bidwell, B. N. et al. Silencing of Irf7 pathways in breast cancer cells promotes bone metastasis through immune escape. Nat Med 18, 1224-1231, doi: 10.1038/nm.2830 (2012).
47 Kadariya, Y. et al. Mice heterozygous for germ-line mutations in methylthioadenosine phosphorylase (MTAP) die prematurely of T-cell lymphoma. Cancer Res 69, 5961-5969, doi: 10.1158/0008-5472.CAN-09-0145 (2009).
48 Hertzog, P. J. Overview. Type I interferons as primers, activators and inhibitors of innate and adaptive immune responses. Immunol Cell Biol 90, 471-473, doi: 10.1038/icb.2012.15 (2012).
49 Vanpouille-Box, C., Demaria, S., Formenti, S. C. & Galluzzi, L. Cytosolic DNA Sensing in Organismal Tumor Control. Cancer Cell 34, 361-378, doi: 10.1016/j.ccell.2018.05.013 (2018).
50 Han, G. et al. 9p21 loss confers a cold tumor immune microenvironment and primary resistance to immune checkpoint therapy. Nat Commun 12, 5606, doi: 10.1038/s41467-021-25894-9 (2021).
51 Kryukov, G. V. et al. MTAP deletion confers enhanced dependency on the PRMT5 arginine methyltransferase in cancer cells. Science 351, 1214-1218, doi: 10.1126/science.aad5214 (2016).
52 Zitvogel, L., Galluzzi, L., Kepp, O., Smyth, M. J. & Kroemer, G. Type I interferons in anticancer immunity. Nat Rev Immunol 15, 405-414, doi: 10.1038/nri3845 (2015).
53 Fuertes, M. B., Woo, S. R., Burnett, B., Fu, Y. X. & Gajewski, T. F. Type I interferon response and innate immune sensing of cancer. Trends Immunol 34, 67-73, doi: 10.1016/j.it.2012.10.004 (2013).
54 Ortiz, A. & Fuchs, S. Y. Anti-metastatic functions of type 1 interferons: Foundation for the adjuvant therapy of cancer. Cytokine 89, 4-11, doi: 10.1016/j.cyto.2016.01.010 (2017).
55 Litvin, O. et al. Interferon alpha/beta Enhances the Cytotoxic Response of MEK Inhibition in Melanoma. Mol Cell 57, 784-796, doi: 10.1016/j.molcel.2014.12.030 (2015).
56 Müller, U. et al. Functional role of type I and type II interferons in antiviral defense. Science 264, 1918-1921, doi: 10.1126/science.8009221 (1994).
57 Fung, K. Y. et al. Interferon-& protects the female reproductive tract from viral and bacterial infection. Science 339, 1088-1092, doi: 10.1126/science. 1233321 (2013).
58 Montesion, M. et al. Somatic HLA Class I Loss Is a Widespread Mechanism of Immune Evasion Which Refines the Use of Tumor Mutational Burden as a Biomarker of Checkpoint Inhibitor Response. Cancer Discov 11, 282-292, doi: 10.1158/2159-8290.Cd-20-0672 (2021).
59 Bakhoum, S. F. & Cantley, L. C. The Multifaceted Role of Chromosomal Instability in Cancer and Its Microenvironment. Cell 174, 1347-1360, doi: 10.1016/j.cell.2018.08.027 (2018).
60 Davoli, T., Uno, H., Wooten, E. C. & Elledge, S. J. Tumor aneuploidy correlates with markers of immune evasion and with reduced response to immunotherapy. Science 355, doi: 10.1126/science.aaf8399 (2017).
61 Ahuja, D., Sáenz-Robles, M. T. & Pipas, J. M. SV40 large T antigen targets multiple cellular pathways to elicit cellular transformation. Oncogene 24, 7729-7745, doi: 10.1038/sj.onc. 1209046 (2005).
62 McGranahan, N. et al. Allele-Specific HLA Loss and Immune Escape in Lung Cancer Evolution. Cell 171, 1259-1271.e1211, doi: 10.1016/j.cell.2017.10.001 (2017).
63 Cerami, E. et al. The cBio cancer genomics portal: an open platform for exploring multidimensional cancer genomics data. Cancer Discov 2, 401-404, doi: 10.1158/2159-8290.Cd-12-0095 (2012).
64 Gao, J. et al. Integrative analysis of complex cancer genomics and clinical profiles using the cBioPortal. Science signaling 6, pl1, doi: 10.1126/scisignal.2004088 (2013).
65 Perez, A. R. et al. GuideScan software for improved single and paired CRISPR guide RNA design. Nat Biotechnol 35, 347-349, doi: 10.1038/nbt.3804 (2017).
66 Gundry, M. C. et al. Highly Efficient Genome Editing of Murine and Human Hematopoietic Progenitor Cells by CRISPR/Cas9. Cell Rep 17, 1453-1461, doi: 10.1016/j.celrep.2016.09.092 (2016).
67 Saborowski, M. et al. A modular and flexible ESC-based mouse model of pancreatic cancer. Genes Dev 28, 85-97, doi: 10.1101/gad.232082.113 (2014).
68 Dow, L. E. et al. Conditional reverse tet-transactivator mouse strains for the efficient induction of TRE-regulated transgenes in mice. PLOS One 9, e95236, doi: 10.1371/journal.pone.0095236 (2014).
69 Dow, L. E. et al. A pipeline for the generation of shRNA transgenic mice. Nat Protoc 7, 374-393, doi: 10.1038/nprot.2011.446 (2012).
70 Love, M. I., Huber, W. & Anders, S. Moderated estimation of fold change and dispersion for RNA-seq data with DESeq2. Genome Biol 15, 550, doi: 10.1186/s13059-014-0550-8 (2014).
71 Mootha, V. K. et al. PGC-1alpha-responsive genes involved in oxidative phosphorylation are coordinately downregulated in human diabetes. Nat Genet 34, 267-273, doi: 10.1038/ng1180 (2003).
72 Subramanian, A. et al. Gene set enrichment analysis: a knowledge-based approach for interpreting genome-wide expression profiles. Proc Natl Acad Sci USA 102, 15545-15550, doi: 10.1073/pnas.0506580102 (2005).
73 Baslan, T. et al. Optimizing sparse sequencing of single cells for highly multiplex copy number profiling. Genome Res 25, 714-724, doi: 10.1101/gr. 188060.114 (2015).
74 Navin, N. et al. Tumour evolution inferred by single-cell sequencing. Nature 472, 90-94, doi: 10.1038/nature09807 (2011).
75 Jassal, B. et al. The reactome pathway knowledgebase. Nucleic Acids Res 48, D498-d503, doi: 10.1093/nar/gkz1031 (2020).
76 Moffitt, R. A. et al. Virtual microdissection identifies distinct tumor-and stroma-specific subtypes of pancreatic ductal adenocarcinoma. Nat Genet 47, 1168-1178, doi: 10.1038/ng.3398 (2015).
77 Chen, E. Y. et al. Enrichr: interactive and collaborative HTML5 gene list enrichment analysis tool. BMC bioinformatics 14, 128, doi: 10.1186/1471-2105-14-128 (2013).
78 Stoeckius, M. et al. Cell Hashing with barcoded antibodies enables multiplexing and doublet detection for single cell genomics. Genome Biol 19, 224, doi: 10.1186/s13059-018-1603-1 (2018).
79 Azizi, E. et al. Single-Cell Map of Diverse Immune Phenotypes in the Breast Tumor Microenvironment. Cell 174, 1293-1308.e1236, doi: 10.1016/j.cell.2018.05.060 (2018).
80 Wolf, F. A., Angerer, P. & Theis, F. J. SCANPY: large-scale single-cell gene expression data analysis. Genome Biol 19, 15, doi: 10.1186/s13059-017-1382-0 (2018).
81 McInnes L, H. J., Melville J. UMAP: Uniform Manifold Approximation and Projection for Dimension Reduction. Arxiv (2018).
82 Levine, J. H. et al. Data-Driven Phenotypic Dissection of AML Reveals Progenitor-like Cells that Correlate with Prognosis. Cell 162, 184-197, doi: 10.1016/j.cell.2015.05.047 (2015).
83 Finak, G. et al. MAST: a flexible statistical framework for assessing transcriptional changes and characterizing heterogeneity in single-cell RNA sequencing data. Genome Biol 16, 278, doi: 10.1186/s13059-015-0844-5 (2015).
84 Duong, E. et al. Type I interferon activates MHC class I-dressed CD11b (+) conventional dendritic cells to promote protective anti-tumor CD8 (+) T cell immunity. Immunity 55, 308-323.e309, doi: 10.1016/j.immuni.2021.10.020 (2022).

1. A method for treating cancer in a patient in need thereof comprising administering to the patient an effective amount of interferon E (IFNE), wherein the patient comprises focal deletions in Cdkn2a and Cdkn2b.

2. A method for enhancing responsiveness to immune checkpoint blockade therapy in a patient in need thereof comprising administering to the patient an effective amount of interferon E (IFNE) and an effective amount of an immune checkpoint inhibitor, wherein the patient comprises focal deletions in Cdkn2a and Cdkn2b.

3. The method of claim 1, wherein the focal deletions in Cdkn2a and Cdkn2b are no more than 0.4 Mb in length.

4. The method of claim 1, wherein the patient further comprises deletions in at least one IFN gene in type I IFN cluster, optionally wherein
the deletions in the type I IFN cluster are no more than 1.3 Mb in length or
the type I IFN cluster comprises IFN-α1, IFN-α2, IFN-α4, IFN-α5, IFN-a6, IFN-α7, IFN-α8, IFN-α10, IFN-α13, IFN-α14, IFN-α16, IFN-α17, IFN-α21, IFNB, IFN-Epsilon, IFN-Kappa, and IFN-Omega.

5. (canceled)

6. (canceled)

7. The method of claim 1, wherein the cancer is lung cancer, pancreatic cancer, head and neck squamous cell cancer, esophageal carcinoma, skin cutaneous melanoma, stomach cancer, glioblastoma, bladder urothelial carcinoma, or brain lower grade glioma, optionally wherein the pancreatic cancer is pancreatic adenocarcinoma (PDAC) or the lung cancer is lung adenocarcinoma (LUAD) or lung squamous cell carcinoma.

8. (canceled)

9. (canceled)

10. The method of claim 2, wherein the immune checkpoint inhibitor is an anti-PD-1 antibody, an anti-PD-L1 antibody, an anti-PD-L2 antibody, an anti-CTLA-4 antibody, an anti-TIM3 antibody, an anti-4-1BB antibody, an anti-CD73 antibody, an anti-GITR antibody, an anti-LAG-3 antibody, ipilimumab, Nivolumab, Pembrolizumab, Atezolizumab, Avelumab, or Durvalumab.

11. The method of claim 1, wherein the IFNE is administered orally, topically, intranasally, systemically, intravenously, subcutaneously, intraperitoneally, intradermally, intraocularly, iontophoretically, transmucosally, or intramuscularly.

12. The method of claim 1, wherein the IFNE comprises an amino acid sequence that is at least 80%, 85%, 90%, 95%, 96%, 97%, 98%, or 99% identical to SEQ ID NO: 64.

13. A donor nucleic acid template including a bicistronic expression cassette comprising a first cistron and a second cistron that are tandemly located, wherein the first cistron encodes a positive selection marker and the second cistron encodes a negative selection marker.

14. The donor nucleic acid template of claim 13, wherein a first artificial protospacer sequence is located upstream of the bicistronic expression cassette and wherein a second artificial protospacer sequence is located at downstream of the bicistronic expression cassette.

15. The donor nucleic acid template of claim 13, wherein the second cistron is located at the 5′ end or the 3′ end of the first cistron.

16. The donor nucleic acid template of claim 13, further comprising a heterologous nucleic acid encoding an enzyme, a bioluminescent protein, a fluorescent protein, and/or a chemiluminescent protein, wherein the heterologous nucleic acid is located upstream or downstream of the bicistronic expression cassette.

17. The donor nucleic acid template of claim 13, wherein the bicistronic expression cassette is operably linked to an inducible promoter or a constitutive promoter.

18. The donor nucleic acid template of claim 13, wherein the positive selection marker is an antibiotic resistance gene.

19. The donor nucleic acid template of claim 13, wherein the positive selection marker comprises neomycin phosphotransferase, hygromycin phosphotransferase, phosphoinothricin acetyltransferase, glyphosate oxidoreductase, adenosine deaminase (ADA), aminoglycoside phosphotransferase, bleomycin, cytosine deaminase, dihydrofolate reductase, histidinol dehydrogenase, puromycin-N-acetyl transferase, thymidine kinase, or xanthine-guanine phosphoribosyltransferase.

20. The donor nucleic acid template of claim 13, wherein the negative selection marker comprises herpes simplex virus thymidine kinase (HSV-TK), rnlA, ypjF, ykfl, ydaS, yjhX, relE, mqsR, toxin CcdB, levansucrase, cytosine deaminase, or diphtheria toxin A (DT-A).

21. The donor nucleic acid template of claim 13, wherein an Internal Ribosome Entry Site (IRES) sequence or a 2A peptide sequence is interspersed between the first cistron and the second cistron, optionally wherein the 2A peptide sequence comprises any one of SEQ ID NOs: 59-62.

22. (canceled)

23. A method for knocking in a genetic alteration at a target gene locus in cells comprising:
(a) contacting cells with a sgRNA-CRISPR enzyme conjugate in vivo under conditions where the sgRNA-CRISPR enzyme conjugate cleaves an endogenous protospacer sequence at the target gene locus in the cells to produce a cleaved target gene locus;
(b) integrating the donor nucleic acid template of claim 14 into the cleaved target gene locus via CRISPR-facilitated homology-directed repair, wherein the donor nucleic acid template comprises a 5′ flanking region and a 3′ flanking region that are homologous to the target gene locus;
(c) enriching cells that stably express the positive selection marker;
(d) contacting the enriched cells of step (c) with a first sgRNA-CRISPR enzyme complex and a second sgRNA-CRISPR enzyme complex in vivo under conditions where the first sgRNA-CRISPR enzyme complex cleaves the first artificial protospacer sequence within the donor nucleic acid template and the second sgRNA-CRISPR enzyme complex cleaves the second artificial protospacer sequence within the donor nucleic acid template to delete the bicistronic expression cassette; and
(e) eliminating cells that stably express the negative selection marker to obtain a cell population comprising the genetic alteration at the target gene locus.

24. A method for knocking in a genetic alteration at a target gene locus in cells comprising:
(a) contacting cells with a first sgRNA-CRISPR enzyme conjugate in vivo under conditions where the sgRNA-CRISPR enzyme conjugate cleaves a first endogenous protospacer sequence at the target gene locus in the cells to produce a cleaved target gene locus;
(b) integrating the donor nucleic acid template of claim 14 into the cleaved target gene locus via CRISPR-facilitated homology-directed repair, wherein the donor nucleic acid template comprises a 5′ flanking region and a 3′ flanking region that are homologous to the target gene locus;
(c) enriching cells that stably express the positive selection marker;
(d) contacting the enriched cells of step (c) with a second sgRNA-CRISPR enzyme complex and a third sgRNA-CRISPR enzyme complex in vivo under conditions where the second sgRNA-CRISPR enzyme complex cleaves the first artificial protospacer sequence within the donor nucleic acid template and the third sgRNA-CRISPR enzyme complex cleaves a second endogenous protospacer sequence at the target gene locus to delete the bicistronic expression cassette, wherein the second endogenous protospacer sequence is located downstream of the bicistronic expression cassette; and
(e) eliminating cells that stably express the negative selection marker to obtain a cell population comprising the genetic alteration at the target gene locus.

25. The method of claim 23, wherein the cells are embryonic stem cells, or fibroblasts.


SEQUENCE LISTING (66 sequences)

SEQ ID NO: 1 - DNA, 59 bp, synthetic construct
  source 1..59: mol_type = other DNA
taatacgactcactataggtgatggggtccggtcctcaagttttagagctagaaatagc

SEQ ID NO: 2 - DNA, 59 bp, synthetic construct
  source 1..59: mol_type = other DNA
taatacgactcactataggaccggccgggtggaactgcggttttagagctagaaatagc

SEQ ID NO: 3 - DNA, 59 bp, synthetic construct
  source 1..59: mol_type = other DNA
taatacgactcactataggttggcctccctcaagatgcggttttagagctagaaatagc

SEQ ID NO: 4 - DNA, 59 bp, synthetic construct
  source 1..59: mol_type = other DNA
taatacgactcactataggccgtaggctaccaaaccagagttttagagctagaaatagc

SEQ ID NO: 5 - DNA, 59 bp, synthetic construct
  source 1..59: mol_type = other DNA
taatacgactcactataggctcgaattcatttctgttcggttttagagctagaaatagc

SEQ ID NO: 6 - DNA, 59 bp, synthetic construct
  source 1..59: mol_type = other DNA
taatacgactcactataggctgtaatatcactctataacgttttagagctagaaatagc

SEQ ID NO: 7 - DNA, 59 bp, synthetic construct
  source 1..59: mol_type = other DNA
taatacgactcactataggtcgcgggagtgaaagccgatgttttagagctagaaatagc

SEQ ID NO: 8 - DNA, 59 bp, synthetic construct
  source 1..59: mol_type = other DNA
taatacgactcactataggcgtcctataattgaagcccggttttagagctagaaatagc

SEQ ID NO: 9 - DNA, 59 bp, synthetic construct
  source 1..59: mol_type = other DNA
taatacgactcactatagggtggtgaaacccgaactcgtgttttagagctagaaatagc

SEQ ID NO: 10 - DNA, 59 bp, synthetic construct
  source 1..59: mol_type = other DNA
taatacgactcactataggacatggttaggctttaccccgttttagagctagaaatagc

SEQ ID NO: 11 - DNA, 59 bp, synthetic construct
  source 1..59: mol_type = other DNA
taatacgactcactatagggcgtactcttgccacttccggttttagagctagaaatagc

SEQ ID NO: 12 - DNA, 59 bp, synthetic construct
  source 1..59: mol_type = other DNA
taatacgactcactataggatagccagatttgcagacgcgttttagagctagaaatagc

SEQ ID NO: 13 - DNA, 59 bp, synthetic construct
  source 1..59: mol_type = other DNA
taatacgactcactatagggatggcgcttgaatactggagttttagagctagaaatagc

SEQ ID NO: 14 - DNA, 59 bp, synthetic construct
  source 1..59: mol_type = other DNA
taatacgactcactataggacataggttggtggcggagtgttttagagctagaaatagc

SEQ ID NO: 15 - DNA, 59 bp, synthetic construct
  source 1..59: mol_type = other DNA
taatacgactcactataggaacctccccacgtaatagaagttttagagctagaaatagc

SEQ ID NO: 16 - DNA, 59 bp, synthetic construct
  source 1..59: mol_type = other DNA
taatacgactcactataggccgccatgtccgccaagagagttttagagctagaaatagc

SEQ ID NO: 17 - DNA, 20 bp, synthetic construct
  source 1..20: mol_type = other DNA
agcaccgactcggtgccact

SEQ ID NO: 18 - DNA, 60 bp, synthetic construct
  source 1..60: mol_type = other DNA
aacagaagtcggtgaatagaatttaaatttcccttgaggaaacgaattctaccgggtagg

SEQ ID NO: 19 - DNA, 60 bp, synthetic construct
  source 1..60: mol_type = other DNA
atcaaagagcttgcctgatgctccctgtgatggggtccggccatagagcccaccgcatcc

SEQ ID NO: 20 - DNA, 60 bp, synthetic construct
  source 1..60: mol_type = other DNA
tataacaggggacaccacctcttccagagaccgtaggctaaacgaattctaccgggtagg

SEQ ID NO: 21 - DNA, 60 bp, synthetic construct
  source 1..60: mol_type = other DNA
gaagatgctgcttatacttggcattagccttctggtttggccatagagcccaccgcatcc

SEQ ID NO: 22 - DNA, 60 bp, synthetic construct
  source 1..60: mol_type = other DNA
gttctgcagtcatgtggaagatggaggccctgcacgctaccaggagctattaattcgcgg

SEQ ID NO: 23 - DNA, 60 bp, synthetic construct
  source 1..60: mol_type = other DNA
gttttcgcagcactggtgtcacctcgtcctgcccaactatccatcttcgggaacatccgg

SEQ ID NO: 24 - DNA, 20 bp, synthetic construct
  source 1..20: mol_type = other DNA
caaggaaagtcaagcccaga

SEQ ID NO: 25 - DNA, 20 bp, synthetic construct
  source 1..20: mol_type = other DNA
ctgtccatctgcacgagact

SEQ ID NO: 26 - DNA, 20 bp, synthetic construct
  source 1..20: mol_type = other DNA
gggcaaaggtgagaaaacaa

SEQ ID NO: 27 - DNA, 20 bp, synthetic construct
  source 1..20: mol_type = other DNA
atccgagatctggaggaggt

SEQ ID NO: 28 - DNA, 20 bp, synthetic construct
  source 1..20: mol_type = other DNA
gggatacccctcaacaggat

SEQ ID NO: 29 - DNA, 20 bp, synthetic construct
  source 1..20: mol_type = other DNA
ctgtccatctgcacgagact

SEQ ID NO: 30 - DNA, 20 bp, synthetic construct
  source 1..20: mol_type = other DNA
gggatacccctcaacaggat

SEQ ID NO: 31 - DNA, 20 bp, synthetic construct
  source 1..20: mol_type = other DNA
cctcataacagtggcctgct

SEQ ID NO: 32 - DNA, 20 bp, synthetic construct
  source 1..20: mol_type = other DNA
gctgtccagggagttagagg

SEQ ID NO: 33 - DNA, 20 bp, synthetic construct
  source 1..20: mol_type = other DNA
taccaggcagaaggagcagt

SEQ ID NO: 34 - DNA, 20 bp, synthetic construct
  source 1..20: mol_type = other DNA
accttgcggtatggagtttg

SEQ ID NO: 35 - DNA, 20 bp, synthetic construct
  source 1..20: mol_type = other DNA
actctcagggttccccaagt

SEQ ID NO: 36 - DNA, 20 bp, synthetic construct
  source 1..20: mol_type = other DNA
aagccacagtagggctcaga

SEQ ID NO: 37 - DNA, 20 bp, synthetic construct
  source 1..20: mol_type = other DNA
taccaggcagaaggagcagt

SEQ ID NO: 38 - DNA, 20 bp, synthetic construct
  source 1..20: mol_type = other DNA
accttgcggtatggagtttg

SEQ ID NO: 39 - DNA, 20 bp, synthetic construct
  source 1..20: mol_type = other DNA
actctcagggttccccaagt

SEQ ID NO: 40 - DNA, 20 bp, synthetic construct
  source 1..20: mol_type = other DNA
aagccacagtagggctcaga

SEQ ID NO: 41 - DNA, 20 bp, synthetic construct
  source 1..20: mol_type = other DNA
tcacatgccccgcccccggc

SEQ ID NO: 42 - DNA, 23 bp, synthetic construct
  source 1..23: mol_type = other DNA
ttagttagcctcccccatctccc

SEQ ID NO: 43 - DNA, 20 bp, synthetic construct
  source 1..20: mol_type = other DNA
ggcatgcctgctattttgat

SEQ ID NO: 44 - DNA, 19 bp, synthetic construct
  source 1..19: mol_type = other DNA
gggagcaacctcggtgata

SEQ ID NO: 45 - DNA, 20 bp, synthetic construct
  source 1..20: mol_type = other DNA
ggctgtattcccctccatcg

SEQ ID NO: 46 - DNA, 22 bp, synthetic construct
  source 1..22: mol_type = other DNA
ccagttggtaacaatgccatgt

SEQ ID NO: 47 - DNA, 20 bp, synthetic construct
  source 1..20: mol_type = other DNA
gggaaattcaacggcacagt

SEQ ID NO: 48 - DNA, 19 bp, synthetic construct
  source 1..19: mol_type = other DNA
agatggtgatgggcttccc

SEQ ID NO: 49 - DNA, 20 bp, synthetic construct
  source 1..20: mol_type = other DNA
acgtaaacggccacaagttc

SEQ ID NO: 50 - DNA, 20 bp, synthetic construct
  source 1..20: mol_type = other DNA
aagtcgtgctgcttcatgtg

SEQ ID NO: 51 - DNA, 21 bp, synthetic construct
  source 1..21: mol_type = other DNA
cagctccaagaaaggacgaac

SEQ ID NO: 52 - DNA, 21 bp, synthetic construct
  source 1..21: mol_type = other DNA
ggcagtgtaactcttctgcat

SEQ ID NO: 53 - DNA, 20 bp, synthetic construct
  source 1..20: mol_type = other DNA
gacagctccctgaaacggtg

SEQ ID NO: 54 - DNA, 21 bp, synthetic construct
  source 1..21: mol_type = other DNA
ggctttctctgttcatccaca

SEQ ID NO: 55 - DNA, 19 bp, synthetic construct
  source 1..19: mol_type = other DNA
tgggcctcaaagggtgagt

SEQ ID NO: 56 - DNA, 22 bp, synthetic construct
  source 1..22: mol_type = other DNA
cgggtcggatgtacttgtcata

SEQ ID NO: 57 - DNA, 19 bp, synthetic construct
  source 1..19: mol_type = other DNA
caggagctgtacggcttcc

SEQ ID NO: 58 - DNA, 23 bp, synthetic construct
  source 1..23: mol_type = other DNA
cctaccttgagtaccttgagcac

SEQ ID NO: 59 - protein, 21 aa, synthetic construct
  VARIANT 1..3: note = This region may be present or absent
GSGEGRGSLLTCGDVEENPGP

SEQ ID NO: 60 - protein, 22 aa, synthetic construct
  VARIANT 1..3: note = This region may be present or absent
GSGATNFSLLKQAGDVEENPGP

SEQ ID NO: 61 - protein, 23 aa, synthetic construct
  VARIANT 1..3: note = This region may be present or absent
GSGQCTNYALLKLAGDVESNPGP

SEQ ID NO: 62 - protein, 25 aa, synthetic construct
  VARIANT 1..3: note = This region may be present or absent
GSGVKQTLNFDLLKLAGDVESNPGP

SEQ ID NO: 63 - DNA, 1501 bp, Homo sapiens
  source 1..1501: mol_type = other DNA
cttagatattaaactgataggataagatataaaataatttaagattgctgatatatgttttaaaattaattatttgctcaagcatttgtgacaatttacagttctaattgaggttttaaatttagtagtttgtaggtattttaagttttgcccctgaattctttataggtgctgataagcctttggtaagttttactccatgaaagactattactgaaaaaaacgtaatctcaataaaagaactttaataagcttgactaaatatttagaaagcacattgtgttcagtgaaactttgtatataatgaatagaataataaaagattatgttggatgactagtctgtaattgcctcaaggaaagcatacaatgaataagttattttggtacttcctcaaaatagccaacacaatagggaaatggagaaaatgtactctgaacaccatgaaaagggaacctgaaaatctaatgtgtaaacttggagaaatgacattagaaaacgaaagcaacaaaagagaacactctccaaaataatctgagatgcatgaaaggcaaacattcactagagctggaatttccctaagtctatgcagggataagtagcatatttgaccttcaccatgattatcaagcacttctttggaactgtgttggtgctgctggcctctaccactatcttctctctagatttgaaactgattatcttccagcaaagacaagtgaatcaagaaagtttaaaactcttgaataagttgcaaaccttgtcaattcagcagtgtctaccacacaggaaaaactttctgcttcctcagaagtctttgagtcctcagcagtaccaaaaaggacacactctggccattctccatgagatgcttcagcagatcttcagcctcttcagggcaaatatttctctggatggttgggaggaaaaccacacggagaaattcctcattcaacttcatcaacagctagaatacctagaagcactcatgggactggaagcagagaagctaagtggtactttgggtagtgataaccttagattacaagttaaaatgtacttccgaaggatccatgattacctggaaaaccaggactacagcacctgtgcctgggccattgtccaagtagaaatcagccgatgtctgttctttgtgttcagtctcacagaaaaactgagcaaacaaggaagacccttgaacgacatgaagcaagagcttactacagagtttagaagcccgaggtaggtggagggactagaggacttctccagacatgattcttcatagagtggtaatacaatttatagtacaatcacattgctttgattttgtgtatatatatatttatctgtgttttaagattgtgcatattgaccacaattgtttttattttgtaatgtggctttatatattctatccattttaaattgtttgtatgtcaaaataaattcattaatatggttgattcttcaaaaaaaaaaaaaaaaaaaaaaaaaaaaa

SEQ ID NO: 64 - protein, 208 aa, Homo sapiens
MIIKHFFGTVLVLLASTTIFSLDLKLIIFQQRQVNQESLKLLNKLQTLSIQQCLPHRKNFLLPQKSLSPQQYQKGHTLAILHEMLQQIFSLFRANISLDGWEENHTEKFLIQLHQQLEYLEALMGLEAEKLSGTLGSDNLRLQVKMYFRRIHDYLENQDYSTCAWAIVQVEISRCLFFVFSLTEKLSKQGRPLNDMKQELTTEFRSPR

SEQ ID NO: 65 - DNA, 51 bp, synthetic construct
  source 1..51: mol_type = other DNA
tctaatttggggtccgagacctcgcaatcttgagggaggccaaggacagtg

SEQ ID NO: 66 - protein, 15 aa, synthetic construct
FGVRDLAILREAKDS